(19) United States
(12) Patent Application Publication
(10) Pub. No.: US 2003/0113925 A1
(43) Pub. Date: Jun. 19, 2003

(54) Nuclear morphology based identification and quantification of white blood cell types using optical bio-disc systems

(76) Inventors: John Francis Gordon, Irvine, CA (US); Gowri Pyapali Selvan, Irvine, CA (US)

Correspondence Address: Donald Bollella, Esq., Chief Patent Counsel, BURSTEIN TECHNOLOGIES, INC., 163 Technology Drive, Irvine, CA, 92618, US

(21) Appl. No.: 10/236,857

(22) Filed: Sep. 6, 2002

Related U.S. Application Data
(63) Continuation-in-part of application No. 09/988,728, filed on Nov. 20, 2001.
(60) Provisional application No. 60/318,026, filed on Sep. 7, 2001.
(60) Provisional application No. 60/322,040, filed on Sep. 11, 2001.
(60) Provisional application No. 60/322,863, filed on Sep. 12, 2001.
(60) Provisional application No. 60/322,527, filed on Sep. 14, 2001.
(60) Provisional application No. 60/326,800, filed on Oct. 3, 2001.
(60) Provisional application No. 60/353,300, filed on Jan. 31, 2002.
(60) Provisional application No. 60/355,644, filed on Feb. 5, 2002.
(60) Provisional application No. 60/358,479, filed on Feb. 19, 2002.
(60) Provisional application No. 60/363,949, filed on Mar. 12, 2002.

(51) Int. Cl. G01N 33/48; G01N 21/63

(52) U.S. Cl. 436/10; 436/63; 436/164; 436/165; 422/82.05; 422/82.09

(57) ABSTRACT

The present invention relates in general to biological assays and diagnostic assays and, in particular, to methods and apparatuses for imaging cells using an optical bio-disc system. More specifically, but without restriction to the particular embodiments hereinafter described in accordance with the best mode of practice, this invention relates to methods for identifying and quantitating cells including white blood cells based on the morphology of their nucleus using stains that absorb electromagnetic radiation at pre-determined wavelengths in conjunction with optical bio discs.

### CROSS-REFERENCE TO RELATED APPLICATIONS

This application is a continuation-in-part of U.S. application Ser. No. 09/988,728 filed Nov. 20, 2001.

This application also claims the benefit of priority from U.S. Provisional Application Serial No. 60/318,026 filed Sep. 7, 2001; No. 60/322,040 filed Sep. 11, 2001; No. 60/322,863 filed Sep. 12, 2001; No. 60/322,527 filed Sep. 14, 2001; No. 60/326,800 filed Oct. 3, 2001; No. 60/353,300 filed Jan. 31, 2002; No. 60/355,644 filed Feb. 5, 2002; No. 60/358,479 filed Feb. 19, 2002; and No. 60/363,949 filed Mar. 12, 2002. These applications are herein incorporated by reference in their entireties.

### BACKGROUND OF THE INVENTION

1. Field of the Invention

This invention relates in general to biological assays and diagnostic assays and, in particular, to methods and apparatuses for imaging blood cell analytes in biological samples. More specifically, but without restriction to the particular embodiments hereinafter described in accordance with the best mode of practice, this invention relates to methods for identifying and quantitating white blood cells based on the morphology of their nucleus using stains that absorb electromagnetic radiation at pre-determined wavelengths in conjunction with optical bio discs. The present invention may be advantageously employed in combination with any of the discs, assays, and systems disclosed in the following commonly assigned and co-pending patent applications: U.S. Provisional Application Serial No. 60/302,757 entitled “Clinical Diagnostic Optical Bio-Disc And Related Methods For Selection And Detection Of Lymphocytes Including Helper-Inducer/Suppressor-Cytotoxic Cells” filed Jul. 3, 2001; U.S. Provisional Application Serial No. 60/306,035 entitled “Quantitative and Qualitative Methods for Cell Isolation and Typing Including Immunophenotyping” filed Jul. 17, 2001; U.S. Provisional Application Serial No. 60/305,993 entitled “Capture Layer Assemblies and Optical Bio-Discs for Immunophenotyping” filed Jul. 17, 2001; U.S. Provisional Application Serial No. 60/306,592 entitled “Methods for Imaging Blood Cells, Blood-Borne Parasites and Pathogens, and Other Biological Matter Including Related Optical Bio-Discs and Drive Assemblies” filed Jul. 19, 2001; and U.S. Provisional Application Serial No. 60/307,263 entitled “Quantitative and Qualitative Methods for Cell Isolation and Typing Including Immunophenotyping” filed Jul. 23, 2001. All of these applications are herein in incorporated by reference.

2. Discussion of the Related Art

Blood count screening is a routine clinical test for normal and several conditions including acute or chronic disease, injury, known anemia's, parasitic diseases, myeloid disorders, leukemia's and during treatment with myelosuppressive drugs. Also as a routine test to secure a baseline for surgical procedures, blood transfusions or as a tool in the diagnosis or therapy of a condition. Blood cell counts are used during diagnosis, treatment, and follow-up to determine the health of the patient. Blood counts by themselves cannot determine whether a person has lymphoma or other diseases. However, these values can determine whether everything is normal and if further tests are required.

A number of research and diagnostic situations require isolation and analysis of specific cells from a mixture of cells. Particularly the source could be blood, spinal fluid, bone marrow, tumor homogenates, lymphoid tissue, and the like. Routine screening of blood, spinal fluid, marrow, tumor tissue, lymph, and the like is required to secure a baseline for surgical procedures, blood transfusions or as a tool in diagnosis, treatment or therapy to determine the health of the patient.

Blood cell counts are also used during diagnosis, treatment, and follow-up to determine the health of the patient. Complete blood count (CBC) is a collection of tests including hemoglobin, hematocrit, mean corpuscular hemoglobin, mean corpuscular hemoglobin concentration, mean corpuscular volume, platelet count, and white blood cell count. Blood count is the enumeration of the red corpuscles and the leukocytes per cubic mm of whole blood.

White Blood Cell Count (WBC, leukocytes) is the total number of white blood cells in a standard sample of blood. In a normal healthy person, typically the WBC counts are 4000 to 10800 cells per microliter (μl). Factors such as exercise, stress, and disease can affect these values. A high WBC may indicate infection, leukemia, or tissue damage. There is an increased risk of infection when the WBC count falls below 1000 cells per microliter.

Leukocyte differential testing is essential to gather information beyond that obtainable from the leukocyte count itself. Leukocyte differential count is used to evaluate newly suspected infection or fever (even if the CBC is normal), suspicion of a disorder associated with abnormalities, an abnormal leukocyte count, suspected leukemia, and other abnormalities such as eosinophilia, monocytosis, or basophilia. Repeated testing for leukocyte or leukocyte differential may be performed in the presence of severe leukopenia (e.g., secondary to drug therapy). During treatment, for example during chemotherapy or radiation therapy, blood counts are very important to determine whether the treatment is depleting healthy blood cells in addition to cancerous cells. Since chemotherapy affects the production of blood cells, it is important to check the amount of various kinds of cells in the blood.

Differential leukocyte counts may be determined by computerized cell counting equipment. Such apparatus determine the total count and the percentages of the five major white cell types. In normal individuals, there are a majority of neutrophils (50-60%), followed by lymphocytes (20-40%), then monocytes (2-9%), with a few eosinophils (1-4%) and basophils (0.5-2%).

The WBC differential count is usually abnormal due to either increased leukocytes that may be due to a reactive or neoplastic response and decreased leukocytes may be due consumption or destruction or bone marrow failure. Conditions like neutropenia, (Neutrophil loss), lymphocytopenia (loss of lymphoctyes), thrombocythemia (platelet depletion, can be life threatening) can be determined by a differential leukocyte count. A differential count also gives an overall picture of blood counts specifically during chemotherapy and radiation therapy treatments that affects the production of blood cells.

Within the category of lymphocytes there are further sub-types of cells. For example, lymphocytes can be broadly divided into T-cells (thymus-derived lymphocytes) and B-cells (bursal-equivalent lymphocytes), which are largely responsible for cell-mediated and humoral immunity respectively. Although morphological characteristics have been used to classify groups within the leukocytes, morphology alone has proved inadequate in distinguishing the many functional capabilities of lymphocyte sub-types. To distinguish lymphocytes with various functions, techniques including analysis by rosetting, immuno-fluorescence microscopy, enzyme histochemistry, and recently, monoclonal antibodies against unique cell surface markers have been developed.

T-cells are often further distinguished by the presence of one of two major cell surface antigens such as CD4 and CD8. Type CD4+ cells are referred to as helper T-cells and are involved in antibody-mediated immunity. These T-cells bind to antigens presented by B-cells and cause the development of a clone of plasma cells which secrete antibodies against the antigenic material. The CD4+ T-cells are also essential for cell-mediated immunity. It is understood that CD4+ T-cells bind to antigen presented by antigen-presenting cells (APCs) such as phagocytic macrophages and dendritic cells, and release lymphokines that attract other immune system cells to the area. The result is inflammation, and the accumulation of cells and molecules that attempt to wall off and destroy the antigenic material.

Type CD8+ T-cells are referred to as cytotoxic or killer T-cells. These T-cells secrete molecules that destroy the cell to which they have bound. This is important in fighting viral infections, since the CD8+ T-cells destroy the infected cells before they can release a fresh crop of viruses that are able to infect other cells.

The estimation of CD4+ and CD8+ T-cells and the ratio of CD4+/CD8+ T-cells is useful to assess the immune health of human patients with immune-compromised diseases. For example, the human immunodeficiency virus (HIV) is a retrovirus with high affinity for the CD4 cell surface antigen, and therefore CD4+ T-cells are potent targets for the virus. Acquired immune deficiency syndrome (AIDS) provides a vivid and tragic illustration of the importance of CD4+ T-cells in immunity. As the disease progresses, the number of CD4+ T-cells declines below its normal range of about 1000 per μl, as the patient's CD8+ T-cells destroy the infected CD4+ T-cells and/or infected CD4+ cells undergo apoptosis or cell suicide. Thus, the ratio of CD4+ to CD8+ T-cells may be used as a prognostic indicator. The U.S. Public Health Service recommends that CD4+ levels be monitored every 3-6 months in all infected persons.

In addition to CD4 and CD8, there are many other cell surface antigens (for example, CD3, CD16, CD19, CD45, and CD56) which can be used to identify sub-types of lymphocytes. The ability to detect these cell surface antigens by antibody techniques has added a new dimension to diagnostic pathology, and a variety of techniques are available for the study of immunophenotypes of hematolymphoid disorders (e.g., AIDS, leukemias, and lymphomas). Conventional microimmuno-assays such as radio-immunoassays (RIA), enzyme-immunoassay (EIA), fluorescence-immunoassay (FIA) use an isotope, an enzyme, or a fluorescent substance in order to detect the presence or absence of corresponding antibodies or antigens, respectively, that react specifically therewith.

The number of platelets in a standard sample of blood typically is 133,000 to 333,000 platelets per microliter (μl). An excess number of platelets is called thrombocythemia. Above normal platelet counts may be due to a reactive response or bone marrow failure. Reactive responses are typically caused by bleeding, infection, neoplasia, and myeloproliferative disorders. Bone marrow failure usually involves loss of blood cells known as pancytopenia. On the other hand, decreased platelet counts are due to immune thrombocytopenia. Thrombocytopenia occurs if the platelet count fall below 30,000, which results in abnormal bleeding. Counts below 5000 are considered life threatening.

A CBC may be done by commercially available manual or electronic instruments that measure hemoglobin level, hematocrit, total leukocyte, and erythrocyte count. Variations may include a platelet count, a leukocyte differential count, and cellular indices. The hematology analyzers are fully automated and results are accurate for cell counts, types of cells in body fluids like CSF, pleural fluid, ascetic fluid, pericardial fluid, and gastric aspiration.

As compared to prior methods and systems, we have developed a simple, miniaturized, ultra-sensitive, inexpensive system for imaging and analyzing cells and their components. This system uses optical bio-discs, related detection assemblies, as well as information and signal processing methods and software.

### SUMMARY OF THE INVENTION

The present invention is designed to image, identify, and quantify unstained/unlabeled, or stained/labeled cells. In addition thereto in preferred embodiments of the present invention, cells are stained using dyes that stain the nucleus of the cell including infrared dyes and fluorescent dyes to enhance the contrast between the cytoplasm and the nucleus of the cell to aid in its identification. This also facilitates visualization of additional information and details of normal and abnormal cells in the samples. Details relating to cell imaging using optical bio-discs are disclosed in U.S. Provisional Application Serial No. 60/260,761, entitled “Methods and Apparatus for Detecting Investigational Features on a Surface of an Optical Disc Assembly” filed Jan. 11, 2001; U.S. Provisional Application Serial No. 60/262,532, entitled “Disklab Diagnostic Platform” filed Jan. 18, 2001; U.S. Provisional Application Serial No. 60/270,095, “Signal Processing Apparatus and Methods for Obtaining Signal Signatures of Investigational Features Detected on a Surface of an Optical Disc Assembly” filed Feb. 20, 2001; and U.S. Provisional Application Serial No. 60/292,108, entitled “Signal Processing Apparatus and Methods for Obtaining Signal Signatures of Investigational Features Detected on a Surface of an Optical Disc Assembly” filed May 18, 2001. All of which are herein incorporated by reference in their entireties.

The test is based upon the principle of optical imaging cells on bio-discs and in special channels located in the disc. The assay may performed within a bio-disc that includes a flow chamber having specific capture agents attached to the solid phase.

Differential cell counting test developed on optical bio-discs identifies various cells in the blood or other body fluids by their light scattering properties which is enhanced by using dyes. This imaging technology also facilitates identification of blood-borne parasites and pathogens using the optical bio-discs. These methods include microscopic analysis or cell detection in a CD-type reader using top detector, bottom detector in conjunction with event counter or cell counter software including hardware counters. Details regarding optical disc systems used for cell imaging and counting are disclosed in, for example, commonly assigned, co-pending U.S. provisional applications serial no. 60/335,123, filed Oct. 24, 2001; No. 60/352,649, filed Jan. 28, 2002; No. 60/353,739, filed Jan. 30, 2002; No. 60/355,09, field Feb. 7, 2002; and No. 60/357,235, filed Feb. 14, 2002; all entitled “Segmented Area Detector for BioDrive and Methods Relating Thereto”. All these applications are herein incorporated by reference in their entireties.

Improved methods and apparatus for carrying out analyses on samples, such as blood, cerebrospinal fluid, sputum, urine, colonocytes, or any other biological source, in a timely, cost efficient, and technically relevant way are provided. In particular, there is a need for easier, more efficient ways to quantify the relative levels of various types of white blood cells, or other cell types, parasites, pathogens, and biological matter. The present invention responds to this need. In addition, this invention relates to imaging blood cells, performing differential white cell counts, and related processing methods and software.

It is an aspect of the invention to provide methods and apparatus for conducting an assay in association with an optical analysis disc to detect and count cells. A further aspect of the invention is to provide methods and apparatuses for conducting assays in association with an optical analysis disc to detect lymphocytes.

According to another aspect of the present invention, there is provided a method that includes providing a sample in or on a disc surface, the disc having encoded information which is readable by an optical reader. This information can be used to control the scanning of the reader relative to the disc.

The present test or assay can be performed in at least two ways. The first method is based upon the principle of optical imaging of blood cells in special channels located on the optical bio-disc. Approximately 5 to 20 microliters of whole blood is injected into specially designed channels on the disc. The images are analyzed with cell recognition software that identifies various leukocyte sub-types and generates a white cell differential count. The second method is based on specific cell capture using cell specific antibodies against a specific cell. In one particular embodiment thereof, antibodies are directed against lymphocytes (CD2, CD19), monocytes (CD14), and eosinophils (CD15), for example. These leukocyte sub-type specific antibodies are assembled/attached to a solid surface within a bio-disc that includes a flow chamber. In other embodiments, capture antibodies are directed against other cells having specific surface markers of interest such as, for example, CD3, CD4, CD8, and CD45.

The disc is loaded into the optical reader, and an incident beam of electromagnetic radiation from a radiation source is directed to the disc. The beam is scanned over the disc by rotating the disc about a central axis and by moving the incident beam in a direction radial to the axis. A beam of electromagnetic radiation either transmitted through or reflected from the disc is detected and analyzed to extract information characteristic of the sample.

Embodiments of the invention also include a disc with a substrate and cap spaced to form a chamber. A sample of material, such as blood with cells, is provided in the chamber. When the disc is rotated, the sample moves past capture zones. The capture zones include capture layers with antibodies or other specific binding partners that bind to antigens such as CD4 and CD8 that are cell surface markers on the cell types of interest. Preferably one test can be used to image CD4 and CD8 and other antigens in a blood sample. According to another aspect of the present invention, there is provided a disc reader for directing light to viewing windows where the capture zones are located, and detecting transmitted or reflected light to identify and count captured cells. These CD4 and CD8 counts, and the ratio between them, are useful for monitoring conditions such as AIDS.

The test sample is preferably provided to a chamber within the disc. A single chamber preferably has multiple capture areas, each of which may have one or more antibodies. In one embodiment, a single channel has multiple capture zones, each with a different type of antibody, and may have capture zones that serve as control zones. These capture zones can be aligned along one or more radii of the disc. Detection methods include detecting transitions in the feature, or imaging the viewing window and using image recognition software to count captured cells. Counting may be direct, such as counting a desired cell; or indirect, such as counting a collection of desired and non-desired cells, counting non-desired cells, and subtracting to obtain a count of desired cells. The capture zone may have one or more layers of antibodies.

When a sample of cells is provided to the disc, the disc can be rotated in one or more stages to move the cells to the capture zones, then to move unbound cells away from the capture zones. The sample may be processed in other ways, e.g., incubated or heated with the light source that is used for detection. Microfluidics can be used to add stain or any other liquids that may be desired for on-disc processing of the sample. This processing is preferably specified in encoded information on the disc in information storage areas. The stored information may be advantageously employed to cause the drive and reader to rotate at desired speeds and for desired times with intermediate other steps, such as incubation.

Micro technologies are particularly valuable in clinical diagnostics for identification of cell types, parasites, pathogens, and other biological matter. The present invention utilizes micro technologies to perform differential white cell counts in whole blood on optical bio-discs. In addition, this invention is directed to imaging blood cells, performing differential white cell counts, and related processing methods and software.

Another test or assay according to the present invention may be performed in at least two ways. The first method is based upon the principle of optical imaging of blood cells in special channels located on the optical bio-disc. Approximately 5 to 20 microliters of whole blood is injected into specially designed channels on the disc. The images are analyzed with cell recognition software that identifies the various leukocyte sub-types and generates a white cell differential count. The second method is based on specific cell capture using cell specific antibodies against specific cell. In this particular embodiment, antibodies are directed against lymphocytes (CD2, CD19), monocytes (CD14), and eosinophils (CD15), for example. As with the related assay discussed above, these leukocyte sub-type specific antibodies are assembled/attached to a solid surface within the bio-disc that includes a flow chamber.

A bio-disc drive assembly is employed to rotate the disc, read and process any encoded information stored on the disc, and analyze the cell capture zones in the flow chamber of the bio-disc. The bio-disc drive is provided with a motor for rotating the bio-disc, a controller for controlling the rate of rotation of the disc, a processor for processing return signals from the disc, and analyzer for analyzing the processed signals. The rotation rate is variable and may be closely controlled both as to speed, time of rotation, and direction of rotation. The bio-disc may also be utilized to write information to the bio-disc either before, during, or after the test material in the flow chamber and target zones is interrogated by the read beam of the drive and analyzed by the analyzer. The bio-disc may include encoded information for controlling the rotation of the disc, providing processing information specific to the type of immunotyping assay to be conducted and for displaying the results on a monitor associated with the bio-drive.

The differential cell count protocols in general and in particular differential white blood cell counting protocols are developed for CD, CD-R, or DVD formats, modified versions of these formats, and alternatives thereto. The read or interrogation beam of the drive detects the various cells in the analysis sample and generates images that can be analyzed with differential cell counter software.

Microscopic methods or sophisticated cell counters are essential to perform these tedious and laborious cell-counting assays. The present method uses optical bio-discs and related assemblies. Optical images of the various leukocyte sub-types free in the analysis chamber or those captured by a specific antibody method are generated and analyzed by cell recognition software programs that identify the various cellular elements in the blood or other body fluids by their light scattering properties. This return light is detected after the light/matter interaction between the incident bean and the sample of interest. The detected return light signal is processed to provide discernable signal signatures or digital IDs. While prior art methods typically require preparation such as cell staining, RBC elimination, or other laborious protocols, embodiments of the present methods may not require any pre-processing of the sample. These methods include microscopic analysis or cell detection in a CD-type or optical disc reader using a top-detector, bottom-detector, event counter, or cell counter.

According one aspect of the present invention, nuclear, chromosomal, vital, infrared, and fluorescent dyes may be employed to enhance the contrast between the cytoplasm and the nucleus of cells. In one preferred embodiment of the present invention, IRDye38 (LI-COR Inc., Lincoln, Nebr.), IR-780 iodide (Sigma-Aldrich), Streptavidin Laser Pro and TO-PRO-5-iodide (Molecular Probes, Eugene, Oreg.), and dd-007 IR dyes are used to label the cell nucleus. This staining process facilitates visualization and identification of various cell types based on the shape of their nuclei. Details regarding imaging of cells using an optical bio-disc is disclosed in U.S. Provisional Application Serial No. 60/293,093, entitled “Disc Drive Assembly For Optical Bio-Discs”, filed May 22, 2001, which is herein incorporated by reference.

The following paragraphs provide a summary of the principal method steps according to certain specific preferred embodiments of the present invention directed to bio-disc manufacturing.

Disc Preparation: Gold reflective discs or transmissive discs are cleaned using an air gun to remove any dust particles. The disc is rinsed twice with iso-propanol, using the spin coater. A 2% polystyrene is spin coated on the disc to give a very thick coating throughout.

Deposition of Chemistry: One embodiment includes a three step deposition protocol that incubates: streptavidin, 30 minute incubated; biotinylated first antibody incubated for 60 minutes; and second capture antibody incubated for 30 minutes. All the steps are done at room temperature in a humidity chamber using stringent washing and drying steps between depositions.

Briefly, 1 μl of 1 mg/ml streptavidin in phosphate buffered saline is layered over each window and incubated for 30 minutes. Excess streptavidin is rinsed off using distilled water and the disc is dried. Biotinylated IgG-dextran complex is prepared by combining equal volumes of biotinylated IgG (125 μg/ml in PBS) and aldehyde-activated dextran (200 μg/ml). Dextran-aldehyde biotinylated-IgG complex is layered over streptavidin in each capture window and incubated for 60 minutes or overnight in a refrigerator. Excess reagent is rinsed off and the disc spun-dry. Specific barcode capture patterns are created by layering capture antibodies on designated spots on the bio-disc slot. For a differential count, anti-neutrophil (CD128 or others), anti-lymphocyte (CD2, CD19, CD56, and others), anti-eosinophil (CD15), anti-monocyte (CD14), anti-basophil (CD63), and anti-platelets (CD32 and CD151) are layered in designated spot of each slot, for example. Table 1 below lists examples of variations of capture patterns for capture layer assembly. Incubate for 30 minutes or overnight in the refrigerator. Assemble the disc using a 25 μm, 50 μm, or 100 μm (50 μm channel requires twice the volume of sample as that needed for 25 μm chamber), straight, U-shaped, or other channel formats and a clear cover disc (for use with a top detector) or reflective cover disc (for use with a bottom detector).

TABLE 1
Capture Layer Assembly and Variations

| Window | 1 | 2 | 3 | 4 | 5 | 6 |
|---|---|---|---|---|---|---|
| 1^{st} Layer | Poly-styrene | Poly-styrene | Poly-styrene | Poly-Styrene | Poly-styrene | Poly-styrene |
| (Active | | | | | | |
| Layer) | | | | | | |
| 2^{nd} Layer | | Streptavidin | Streptavidin | Streptavidin | Streptavidin | Streptavidin |
| Secondary | | B-anti- | B-anti- | B-anti- | B-anti- | B-anti- |
| Antibody | | Mouse | Mouse | Mouse | Mouse | Mouse |
| | IgG+ DCHO | IgG+ DCHO | IgG+ DCHO | IgG+ DCHO | IgG + DCHO | |
| Primary | Reference | Lymphocyte | Neutrophil | Eosinophil | Basophil | Monocyte |
| Antibody | Dot | Specific | Specific | Specific | Specific | Specific |
| | | antibody | antibody | Antibody | antibody | antibody |

Disc Leak-Checking and Blocking Non Specific Binding of Undesired Cells: Since blood, a biohazardous material, is being analyzed, the discs are leak checked, as part of the quality control manufacturing aspects of the present invention, to make sure none of the chambers leak during spinning of the disc with the sample in situ. Each channel is preferably filled with StabilGuard, a commercially available blocking agent, and blocked for at an hour. The disc is spun at 5000 rpm for 5 minutes and inspected for leaks and disc stability. After checking for leaks, the disc is placed in a vacuum chamber for 24 hours. After vacuuming, the chambers filled with PBS buffer or empty are placed in a vacuum pouch and stored in refrigerator until use.

Isolation of Buffy-coat Layer from Whole Blood: Buffy coat is prepared by centrifuging defibrinated venous blood in a centrifuge tube for 25 minutes at 2800 rpm. The supernatant plasma is carefully removed with a fine pipette. Then pipette the underlying white layer that contains the leukocytes and the platelets. An alternate method to obtain the buffy coat from the blood without centrifugation is to allow the blood to sediment with sedimentation-enhancing agents such as fibrinogen, dextran, gum acacia, Ficoll, or methylcellulose. Boyum's reagent (methylcellulose and sodium metrizoate) is particularly suitable for obtaining leukocyte preparation without any red cell contamination. Alternatively the lymphocytes may be isolated form whole blood by positive or negative selection, or lysis methods.

Assay on Disc—Description of Base Technology: One preferred embodiment of the differential white cell count disc test includes three individual components, (1) base disc including the chemistry, (2) channel layer, and (3) cover disc.

Buffy coat or whole blood, preferably diluted in PBS, is injected into the disc chamber, the inlet and outlet ports of the chamber are sealed with tape and the disc is incubated for a desired time preferably at room temperature. For the first method, a given area (e.g., one millimeter square in area) on the disc is scanned using the standard 780 nm laser of the optical drive with the top or bottom detector. Related cell recognition software developed by assignee and disclosed in U.S. Provisional Application Serial No. 60/363,949 entitled “Methods for Differential Cell Counts Including Leukocytes and Use of Optical Bio-Disc for Performing Same” filed Mar. 12, 2002 and U.S. Provisional Application Serial No. 60/404,921 entitled “Methods For Differential Cell Counts Including Related Apparatus and Software For Performing Same” filed Aug. 21, 2002, is automated to give a differential count from the captured image which is preferably equal to a millimeter square, for example. For the second method, the disc is scanned using the standard 780 nm laser to image the capture zone which may include lymphocytes, neutrophils, basophils, eosinophils, monocytes, and platelets. The cell recognition software developed by assignee is automated to perform the following routines: (a) centrifuge the disc to spin off excess unbound cells, (b) image an specific area or specific capture zones, and (c) data processing that includes counting the specifically captured cells in each capture zone and deriving the numbers of different sub-sets of leukocytes.

During the processing step, the recognition software reads across each capture zone and marks cells it encounters. Following processing data from each capture zone, the software displays the number of lymphocytes, neutrophils, basophils, eosinophils, monocytes, and platelets zones per micro liter volume of blood. The entire process takes about 10-15 minutes from inserting the disc into the optical drive to displaying to results of interest.

Related disclosure associated with the present invention is also presented in U.S. Provisional Application Serial No. 60/307,262 entitled “Capture Layer Assemblies and Optical Bio-Discs for Immunophenotyping” filed Jul. 23, 2001; U.S. Provisional Application Serial No. 60/307,264 entitled “Methods for Imaging Blood Cells, Blood-Borne Parasites and Pathogens, and Other Biological Matter Including Related Optical Bio-Discs and Drive Assemblies” filed Jul. 23, 2001; U.S. Provisional Application Serial No. 60/307,562 entitled “Optical Analysis Discs Including Fluidic Circuits for Optical Imaging and Quantitative Evaluation of Blood Cells Including Lymphocytes” filed Jul. 23, 2001; and U.S. Provisional Application Serial No. 60/307,487 entitled “Methods for Differential Cell Counts Including Leukocytes and Use of Optical Bio-Disc for Performing Same” filed Jul. 24, 2001, all of which are herein incorporated by reference.

The following paragraphs provided a summary of the principal elements of the disc specifications according to certain specific preferred embodiments of the present.

Tracking Design: In one preferred embodiment of the present invention, the disc is a forward Wobble Set FDL21:13707 or FDL21:1270 coating with 300 nm of gold. On this reflective disc, oval data windows of size 2×1 mm are etched out by Lithography. “U” shaped channels are used to create chambers that are 25 μm in height. It takes about 7 μl of sample to fill the entire chamber including the inlet and outlet ports. A 4-window/4-channel format may be preferably used. However on the transmissive disc, no data windows are etched, and the entire disc is available for use.

Adhesive and Bonding: In one preferred embodiment, the adhesive or channel layer including the present “U” shaped fluidic circuits is made from Fraylock adhesive DBL 201 Rev C 3M94661. Alternatively straight channels are used to create the chambers.

Cover Disc: Clear disc, fully reflective with 48 sample inlets with a diameter of 0.040 inches location equidistant at radius 26 mm are used in one specific embodiment of the present disc assembly.

Data Capture and Processing: The data disc is scanned and read with the software at a preferred speed of ×4 and a sample rate of 2.67 MHz using assignee's cell recognition software.

Software: The present invention further includes processing methods and related cell recognition and imaging software. This software is directed to conducting and displaying cell counts and differential cell counts. The present software may be stored on the optical bio-disc, in the optical disc drive reader device, or alternatively only accessible by the optical reader from a secured server. This server may be implemented in a computing network such as a Local Area Network (LAN), a Wide Area Network (WAN), or otherwise made available over the Internet under prescribed terms and conditions. Such distribution methods are disclosed in commonly assigned U.S. Provisional Application No. 60/246,824 entitled “Interactive Method and System for Analyzing Biological Samples and Processing Related Medical Information Using Specially Prepared Bio-Optical Disc, Optical Disc Drive, and Internet Connections” filed Nov. 8, 2000 and related U.S. patent application Ser. No. 09/986,078 entitled “Interactive System for Analyzing Biological Samples and Processing Related Information and the Use Thereof” filed Nov. 7, 2001, both of which are herein incorporated by reference in their entireties.

Materials: The materials employed to practice different preferred embodiments disclosed herein include a forward wobble gold metalized photo-resist disc, a transmissive gold metalized disc, pipettes and tips, spin coater, centrifuge, swing-out rotor, Vacutainer™ CPT tubes with an anti-coagulant such as sodium citrate or ethylene diamine tetra acetic acid (EDTA), humidity chamber, disc press, adhesive, cover disc, clear cover disc, tape or equivalent, vacuum apparatus, yellow tips, and vacuum chamber.

Reagents: The reagents employed in performing the cell counts according to certain methods of the present invention include phosphate buffered saline, isopropyl alcohol, distilled water, and StabilGuard.

It is an object of the present invention to overcome limitations in the known art. Another object of the present invention is to adapt a known optical disc system to perform differential white cell counts in whole blood on optical bio-discs. It is a further object of the present invention to image blood cells and perform differential white cell counts.

Technical aspects related to the present invention are also in U.S. Provisional Application Serial No. 60/307,489 entitled “Optical Analysis Discs Including Microfluidic Circuits for Performing Cell Counts” filed Jul. 24, 2001; U.S. Provisional Application Serial No. 60/307,825 entitled “Methods for Reducing Non-Specific Binding of Cells on Optical Bio-Discs Utilizing Charged Matter Including Heparin, Plasma, or Poly-Lysine” filed Jul. 25, 2001; U.S. Provisional Application Serial No. 60/307,762 entitled “Methods for Reducing Non-Specific Binding of Cells on Optical Bio-Discs Utilizing Blocking Agents” filed Jul. 25, 2001; U.S. Provisional Application Serial No. 60/307,764 entitled “Methods for Reducing Bubbles in Fluidic Chambers Using Polyvinyl Alcohol and Related Techniques for Achieving Same in Optical Bio-Discs” filed Jul. 25, 2001; U.S. Provisional Application Serial No. 60/308,214 entitled “Sealing Methods for Containment of Hazardous Biological Materials within Optical Analysis Disc Assemblies” filed Jul. 27, 2001; and U.S. Provisional Application Serial No. 60/308,197 entitled “Methods for Calculating Qualitative and Quantitative Ratios of Helper/Inducer-Suppressor/Cytotoxic T-Lymphocytes Using Optical Bio-Disc Platform” filed Jul. 27, 2001, all of which are herein incorporated-by reference in their entireties.

The present invention is further directed to staining the nuclei of cells with dyes that absorb incident light at a predetermined wavelength and imaging these cells using an optical disc system to identify and quantify various cell types in the sample based on the morphology of their nucleus.

More particularly now, the present invention is directed to a method of conducting an assay employing an optical disc and disc drive. This method includes the steps of (1) providing a sample of cells on a disc surface in a chamber in a disc, the chamber including at least one capture zone with a capture agent, (2) loading the disc into an optical reader, (3) rotating the optical disc, (4) directing an incident beam of electromagnetic radiation to the capture zone, (5) detecting a beam of electromagnetic radiation formed after interacting with the disc at the capture zone, (6) converting the detected beam into an output signal, and (7) analyzing the output signal to extract therefrom information relating to the number and type of cells captured at the capture zone.

According to a different aspect of the present invention, there is provided an optical disc and drive system for receiving a sample. This system a disc including a substrate, a cap parallel to the substrate, and a chamber defined between the cap and substrate including capture zones. A capture layer is positioned over the substrate at the capture zones. A first capture zone has first cell capture agents and a second capture zone has a second cell capture agents. The system further includes a light source for directing light toward the disc at the capture zones, a detector for detecting light reflected from or transmitted through the disc at the capture zones and providing a signal, and a processor for using the signal to distinguish the nuclei of captured cells and count items in the sample bound to the capture molecules.

In accordance with another aspect of the present invention, there is provided a method of performing a white blood cell count employing an optical disc and disc drive. This particular method includes the steps of (1) providing a blood sample in a first tube, the first tube containing a separation gradient, (2) rotating the first tube at a time and speed sufficient to separate the blood sample into layers, and (3) isolating a white blood cell layer from the separated blood sample. This specific method continues with (4) resuspending the white blood cell layer thereby forming a white blood cell suspension, (5) providing a sample of the white blood cell suspension on an optical disc surface, the surface including at least one capture zone with at least one capture agent, (6) loading the optical disc into an optical reader, and (7) rotating the optical disc. This method then concludes with the steps of (8) directing an incident beam of electromagnetic radiation to a capture zone, (9) detecting a beam of electromagnetic radiation formed after interacting with the disc at the capture zone, (10) converting the detected beam into an output signal, and (11) analyzing the output signal to extract therefrom information relating to the morphology of the nuclei of the cells captured at the capture zone. In one specific embodiment, this method may include the additional steps of directing the sample of white blood cells into proximity with the capture agents, incubating the cells in the presence of the capture agents, allowing the cells to specifically bind to the capture agents, and staining nucleus of the cells with a stain that absorbs at or near the wavelength of the beam of electromagnetic radiation.

In the above specific embodiment, this method further includes the step of analyzing the number of cells captured of a specific type to thereby determine a concentration of the specific cell type in the sample. The step of analyzing the morphology of the nuclei of captured cells, in this method, may include detecting changes in the level of light reflected from or transmitted through the disc. This method also includes the step of counting the number of cells of a specific cell type using image recognition to distinguish cells types by nucleus morphology to distinguishing, for example, one type of white blood cell nucleus from another. Dyes for cell staining may be used to enhance image recognition. These dyes may include vital dyes, fluorescent dyes, infrared and near-infrared dyes. The vital dyes may be Leishman's, acridine orange, or Zynostain. Furthermore, the step of rotating the optical disc may include rotating the disc for a sufficient period of time at a sufficient speed so that the cells have an opportunity to bind with the at least one capture agent. This rotating step further includes rotating for a sufficient period of time at a sufficient speed so that unbound cells are moved away from the capture zones. This step may be done at a single speed or multiple speeds. This particular method also may include the step of counting the captured cells in each of the capture zones and providing an output including cell counts.

In accordance with yet another aspect of the present invention, there is provided another method of making an optical assay disc for performing a differential white blood cell count based on nucleus morphology to distinguish cell types. This method includes the steps of providing a substrate, coating the substrate with an active layer, providing a cross-linker on the active layer to thereby create one or more capture zones, allowing the cross-linker to bind to the active layer, removing excess cross-linker from the capture zones, and attaching a cap portion to the active layer to form a channel adapted to receive a suspension of cells and a dye solution for staining the nuclei of captured cells. The cross-linker used in this method may bind with oligossacharides on the surface of cells. This cross-linker may be lectin.

According to another aspect of the present invention there is provided a method of using the disc made according the above method. This specific method of using the pre-made disc includes the steps of depositing a sample comprising white blood cells into the channel, allowing the white blood cells to bind to the cross-linker within the capture zones, removing unbound cells from the capture zones, and loading the disc into an optical reader. The present method of using a pre-made disc further includes the steps of directing an incident beam of electromagnetic radiation to the capture zone, detecting a beam of electromagnetic radiation formed after interacting with the disc and the sample of cells on the disc surface, converting the detected beam into an output signal, and analyzing the output signal to extract therefrom information relating to the number and type of cells captured at the capture zone. This method may further involve the step of staining the sample of cells with a stain that absorbs light at a pre-determined wavelength wherein the pre-determined wavelength is at or near the wavelength of the beam of electromagnetic radiation.

In accordance with still another aspect of the current invention, there is disclosed yet another method of analyzing white blood cells in a test sample. This method involves the steps of (1) loading a plurality of white blood cells into an optical bio-disc, (2) capturing the white blood cells in designated target zones by use of capture agents having specificity for particular cell surface markers, (3) staining the nuclei of the captured white blood cells with a dye, (4) directing an incident beam of light toward the captured white blood cells, and (5) allowing the incident beam of light to interact with the stained nuclei of the captured white blood cells to thereby form a return beam of light carrying information regarding the morphology of the nuclei. This method further involves (6) detecting the return beam of light, (7) converting the detected return beam into an output signal, and (8) analyzing the output signal to extract therefrom information relating to the morphology of the nuclei of the cells captured at the capture zone. The dye used in this method may include vital, vital nuclear, nuclear, DNA, chromosomal, fluorescent, infrared, near-infrared, UV, and visible dyes. This particular method may also include the further step of counting the number of captured white blood cells of a specific type wherein the specific types of white blood cells include neutrophils, monocytes, lymphocytes, eosinophils, and basophils. The present method also involves the use of an optical bio-disc employed for performing the white blood cell analysis in accordance with this method.

According to yet another aspect of the present invention, there is provided a method of conducting an assay employing an optical disc and disc drive, the method comprising the steps of providing a sample of cells on a disc surface, loading the disc into an optical reader, rotating the optical disc, directing an incident beam of electromagnetic radiation to the capture zone, detecting a beam of electromagnetic radiation formed after interacting with the disc and the sample of cells on the disc surface, converting the detected beam into an output signal; and analyzing the output signal to extract therefrom information relating to the number and type of cells captured at the capture zone. This method further includes the step of staining the sample of cells with a dye that absorbs light at a pre-determined wavelength wherein the pre-determined wavelength is at or near the wavelength of the beam of electromagnetic radiation and the dye absorbs light within the infrared spectral range. The dye may absorb light at near-infrared, infrared (IR), ultra violet (UV), and visible (VIS) spectral ranges. Moreover, the beam of electromagnetic radiation may a wavelength within 10 nm of the absorbance wavelength of the dye. The dye used to stain the cells may include, for example, LI-COR IRDye38, TO-PRO-5-iodide, IR-780 iodide, Laser Pro IR, dd-007, Zynostain, idocyanine green, copper phthalocyanine, 3,3′-diethylthiatricarbocyanine iodide (DTTCI), 3,3′-diethyloxatricarbocyanine iodide (DOTCI), 3,3′-diethylthiadicarbocyanine iodide (DTDCI), and 3,3′-diethyloxadicarbocyanine iodide (DODCI). These dyes may be designed to specifically label pre-determined compartments of the sample of cells on the disc surface including, but not limited to the nucleus, nucleolus, nuclear envelope, golgi apparatus, endoplasmic reticulum, mitochondria, vacuoles, peroxisomes, microtubules, centrioles, ribosomes, cell membrane, and cell wall. Furthermore, the step of providing a sample of cells on a disc surface may be performed by smearing the sample of cells to thereby create a monolayer of cells on the disc surface.

The above described methods and apparatus can have one or more advantages which include, but are not limited to, simple and quick on-disc processing without the necessity of an experienced technician to run the test, small sample volumes, use of inexpensive materials, and use of known optical disc formats and drive manufacturing. These and other features and advantages will be better understood by reference to the following detailed description when taken in conjunction with the accompanying drawing figures and technical examples.

### BRIEF DESCRIPTION OF THE DRAWINGS

Further objects of the present invention together with additional features contributing thereto and advantages accruing therefrom will be apparent from the following description of the preferred embodiments of the invention which are shown in the accompanying drawing figures with like reference numerals indicating like components throughout, wherein:

FIG. 1 is a pictorial representation of a bio-disc system according to the present invention;

FIG. 2 is an exploded perspective view of a reflective bio-disc as utilized in conjunction with the present invention;

FIG. 3 is a top plan view of the disc shown in FIG. 2;

FIG. 4 is a perspective view of the disc illustrated in FIG. 2 with cut-away sections showing the different layers of the disc;

FIG. 5 is an exploded perspective view of a transmissive bio-disc as employed in conjunction with the present invention;

FIG. 6 is a perspective view representing the disc shown in FIG. 5 with a cut-away section illustrating the functional aspects of a semi-reflective layer of the disc;

FIG. 7 is a graphical representation showing the relationship between thickness and transmission of a thin gold film;

FIG. 8 is a top plan view of the disc shown in FIG. 5;

FIG. 9 is a perspective view of the disc illustrated in FIG. 5 with cut-away sections showing the different layers of the disc including the type of semi-reflective layer shown in FIG. 6;

FIG. 10 is a perspective and block diagram representation illustrating the system of FIG. 1 in more detail;

FIG. 11 is a partial cross sectional view taken perpendicular to a radius of the reflective optical bio-disc illustrated in FIGS. 2, 3 and 4 showing a flow channel formed therein;

FIG. 12 is a partial cross sectional view taken perpendicular to a radius of the transmissive optical bio-disc illustrated in FIGS. 5, 8 and 9 showing a flow channel formed therein and a top detector;

FIG. 13 is a partial longitudinal cross sectional view of the reflective optical bio-disc shown in FIGS. 2, 3 and 4 illustrating a wobble groove formed therein;

FIG. 14 is a partial longitudinal cross sectional view of the transmissive optical bio-disc illustrated in FIGS. 5, 8 and 9 showing a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17A is a pictorial flow chart showing the analysis of a blood sample using methods of the invention;

FIG. 17B is a pictorial detail view showing the attachment of antibodies to a white blood cell for use with the disc illustrated in FIG. 17A;

FIG. 18A is a pictorial representation of streptavidin;

FIG. 18B is a pictorial representation of biotin;

FIG. 18C is a pictorial representation of the cross-linking system consisting of streptavidin and biotin;

FIG. 18D is a pictorial representation of a secondary antibody;

FIG. 18E is a pictorial representation of a biotinylated secondary antibody;

FIG. 18F is a pictorial representation of a primary antibody;

FIG. 18G is a pictorial representation of a biotinylated primary antibody;

FIG. 18H is a pictorial representation of a CD4⁺ cell showing four CD4 surface antigens;

FIG. 18I is a pictorial representation of a CD8⁺ cell showing four CD8 surface antigens;

FIG. 18J is a pictorial representation showing secondary antibodies bound to aldehyde-activated dextran;

FIG. 18K is a cross-sectional pictorial representation of FIG. 18J;

FIGS. 19A and 19B are pictorial representations showing cell capture by a primary antibody that is bound to a substrate by a cross-linking system in a first implementation of the invention;

FIGS. 19C and 19D are pictorial representations showing cell capture by a primary antibody that is directly bound to a substrate in a first implementation of the invention;

FIGS. 20A-20I are cross-sectional side views showing embodiments of a first implementation of a method of depositing capture agents onto the capture zones of a reflective bio-disc using a cross-linking system according to the present invention;

FIG. 21 is an alternate embodiment of the reflective disc shown in FIGS. 20A-20I without use of the cross-linking system;

FIG. 22 shows the capture chemistries utilized in FIGS. 20A-20I as implemented in a transmissive disc format;

FIGS. 23A and 23B are pictorial representations showing cell capture by a primary antibody that is bound to a secondary antibody, which is bound to a substrate by a cross-linking system in a second implementation of the invention;

FIGS. 23C and 23D are pictorial representations showing cell capture by a primary antibody that is bound to a secondary antibody, which is directly bound to a substrate in a second implementation of the invention;

FIGS. 24A-24L are cross-sectional side views showing embodiments of a second implementation of a method of depositing capture agents onto the capture zones of a reflective bio-disc using primary and secondary capture antibodies and a cross-linking system according to the present invention;

FIG. 25 is an alternate embodiment of the reflective disc shown in FIGS. 24A-24L without use of the cross-linking system;

FIG. 26 shows the capture chemistries utilized in FIGS. 24A-24L as implemented in a transmissive disc format;

FIGS. 27A and 27B are pictorial representations showing cell capture by a primary antibody that is bound to a secondary antibody, which is bound to a substrate by a strand of DCHO in a second implementation of the invention;

FIGS. 27C and 27D are pictorial representations showing cell capture by a primary antibody that is bound directly to a substrate by a strand of DCHO in a second implementation of the invention;

FIG. 28A is a pictorial flow diagram showing preparation of antibody-DCHO complexes;

FIGS. 28B-28J are cross-sectional side views showing embodiments of a second implementation of a method of depositing capture agents onto the capture zones of a reflective bio-disc using primary and secondary antibodies and a strand of DCHO;

FIG. 29 is an alternate embodiment of the reflective disc shown in FIGS. 28B-28J without use of secondary antibodies;

FIG. 30 shows the capture chemistries utilized in FIGS. 28B-28J as implemented in a transmissive disc format;

FIG. 31A is a top plan view of an optical bio-disc showing four fluidic circuits each having several capture zones for a specific cell surface marker and a negative control zone;

FIGS. 31B and 31C are pictorial representations showing capture of a primary antibody-cell complex by a secondary antibody that is bound to a substrate by a cross-linking system in a third implementation of the invention;

FIGS. 31D and 31E are pictorial representations showing capture of a primary antibody-cell complex by a secondary antibody that is directly bound to a substrate in a third implementation of the invention;

FIGS. 32A-32I are cross-sectional side views showing embodiments of a third implementation of a method of depositing capture agents onto the capture zones of a reflective bio-disc using a cross-linking system;

FIG. 33 is an alternate embodiment of the reflective disc shown in FIGS. 32A-32I without use of the cross-linking system;

FIG. 34 shows the capture chemistries utilized in FIGS. 32A-32I as implemented in a transmissive disc format;

FIG. 35A is a top plan view of an optical bio-disc showing four fluidic circuits each having several capture zones for different cell surface markers and a negative control zone;

FIGS. 35B, 35C, and 35D are cross-sectional side views of a reflective optical bio-disc showing embodiments of a first implementation of a method of blood sample analysis using a cross-linking system;

FIG. 36 is an alternate embodiment of the reflective disc shown in FIGS. 35B, 35C, and 35D without use of the cross-linking system;

FIG. 37 shows the capture chemistries utilized in FIGS. 35B, 35C, and 35D as implemented in a transmissive disc format;

FIGS. 38A, 38B, and 38C are cross-sectional side views of a reflective optical bio-disc showing embodiments of a second implementation of a method of blood sample analysis using primary and secondary capture antibodies and a cross-linking system;

FIG. 39 is an alternate embodiment of the reflective disc shown in FIGS. 38A, 38B, and 38C without use of the cross-linking system;

FIG. 40 shows the capture chemistries utilized in FIGS. 38A, 38B, and 38C as implemented in a transmissive disc format;

FIGS. 41A, 41B, and 41C are cross-sectional side views of a reflective optical bio-disc showing embodiments of a third implementation of a method of blood sample analysis using primary and secondary antibodies and a strand of DCHO;

FIG. 42 is an alternate embodiment of the reflective disc shown in FIGS. 41A, 41B, and 41C without use of secondary antibodies;

FIG. 43 shows the capture chemistries utilized in FIGS. 41A, 41B, and 41C as implemented in a transmissive disc format;

FIG. 44A is a pictorial flow diagram showing preparation of primary antibody-cell complexes;

FIGS. 44B, 44C, and 44D are cross-sectional side views of a reflective optical bio-disc showing embodiments of a fourth implementation of a method of blood sample analysis using primary and secondary capture antibodies and a cross-linking system;

FIG. 45 is an alternate embodiment of the reflective disc shown in FIGS. 44B, 44C, and 44D without use of the cross-linking system;

FIG. 46 shows the capture chemistries utilized in FIGS. 44B, 44C, and 44D as implemented in a transmissive disc format;

FIG. 47 is a pictorial diagram of an optical disc having chambers to illustrate a bar code technique according to an embodiment of the present invention;

FIG. 48A is an illustration of results obtained from an assay using the bar code format according to an embodiment of the present invention;

FIG. 48B shows corresponding microscope and disc images for CD4, CD8, and control regions;

FIG. 49 shows a larger view of corresponding microscope and disc images to illustrate the results obtainable from the methods and apparatus of the present invention;

FIGS. 50 and 51 illustrate the use of image recognition according to an embodiment of the present invention;

FIG. 52 is illustrative screen shot of expected output from the bar code according to an embodiment of the present invention;

FIG. 53 shows a method for going from captured cells to usable output according to an embodiment of the present invention;

FIG. 54 is a pictorial graphical representation of the transformation of a sampled analog signal to a corresponding digital signal that is stored as a one-dimensional array;

FIG. 55 is a perspective view of an optical disc with an enlarged detailed view of an indicated section showing a captured white blood cell positioned relative to the tracks of the bio-disc yielding a signal-containing beam after interacting with an incident beam;

FIG. 56A is a graphical representation of a white blood cell positioned relative to the tracks of an optical bio-disc according to the present invention;

FIG. 56B is a series of signature traces derived from the white blood cell of FIG. 56A according to the present invention;

FIG. 57 is a graphical representation illustrating the relationship between FIGS. 57A, 57B, 57C, and 57D;

FIGS. 57A, 57B, 57C, and 57D, when taken together, form a pictorial graphical representation of transformation of the signature traces from FIG. 56B into digital signals that are stored as one-dimensional arrays and combined into a two-dimensional array for data input;

FIG. 58 is a logic flow chart depicting the principal steps for data evaluation according to processing methods and computational algorithms related to the present invention;

FIG. 59 is an illustration of the red blood cells and white blood cells including the various types of white blood cells and their unique anatomical features;

FIG. 60 is an image of white blood cells stained with an infra red dye collected using an optical disc reader; and

FIG. 61 is an enlarged image of some white blood cells shown in FIG. 60.

### DETAILED DESCRIPTION OF THE INVENTION

The present invention is directed to disc drive systems, optical bio-discs, cellular assays and related cell counting methods, image processing techniques, and related software. Each of these aspects of the present invention is discussed below in further detail.

Aspects of the present invention relating to the disc drive systems, optical bio-discs, cellular assays and related cell counting methods, image processing techniques, and related software disclosed herein are also presented in U.S. Provisional Application Serial No. 60/338,679 entitled “Quantitative and Qualitative Methods for Cell Isolation and Typing Including Immunophenotyping” filed Nov. 13, 2001; U.S. Provisional Application Serial No. 60/332,001 entitled “Capture Layer Assemblies and Optical Bio-Discs for Immunophenotyping” filed Nov. 14, 2001; U.S. Provisional Application Serial No. 60/334,131 entitled “Methods for Calculating Qualitative and Quantitative Ratios of Helper/Inducer-Suppressor/Cytotoxic T-Lymphocytes Using Optical Bio-Disc Platform” filed Nov. 30, 2001; U.S. Provisional Application Serial No. 60/349,392 entitled “RBC Sieving Protocol Evaluations of Helper/Inducer-Suppressor/Cytotoxic T-Lymphocytes Using Whole Blood and Related Optical Bio-Disc” filed Jan. 17, 2002; U.S. Provisional Application Serial No. 60/349,449 entitled “RBC Lysis Protocol Evaluations of Helper/Inducer-Suppressor/Cytotoxic T-Lymphocytes Using Whole Blood and Related Optical Bio-Disc” filed Jan. 18, 2002; U.S. Provisional Application Serial No. 60/353,300 entitled “Methods for Differential Cell Counts Including Leukocytes and Use of Optical Bio-Disc for Performing Same” filed Jan. 31, 2002; U.S. Provisional Application Serial No. 60/355,644 entitled “Cluster Designation Assays Performed on Optical Bio-Disc Including Equi-Radial Analysis Zones” filed Feb. 5, 2002; U.S. Provisional Application Serial No. 60/358,479 entitled “Cluster Designation Assays Performed on Optical Bio-Disc Including Equi-Radial Analysis Zones and Related Systems and Methods” filed Feb. 19, 2002; U.S. Provisional Application Serial No. 60/363,949 entitled. “Methods for Differential Cell Counts Including Leukocytes and Use of Optical Bio-Disc for Performing Same” filed Mar. 12, 2002; and U.S. Provisional Application Serial No. 60/404,921 entitled “Methods For Differential Cell Counts Including Related Apparatus and Software For Performing Same” filed Aug. 21, 2002, all of which are herein incorporated by reference.

Drive System and Related Discs

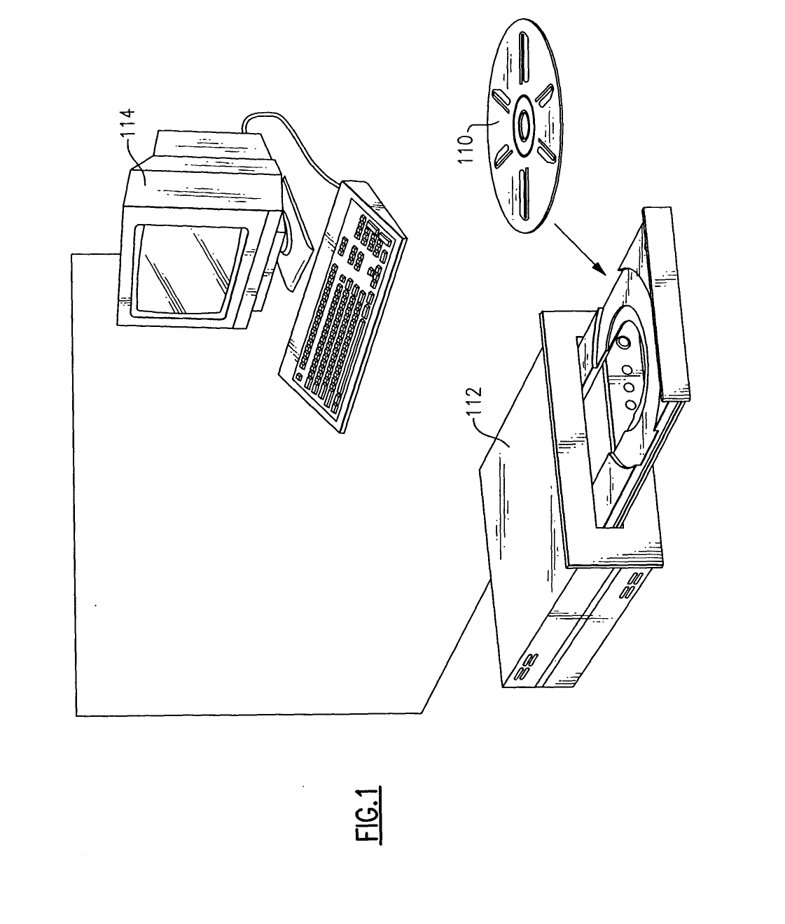

FIG. 1 is a perspective view of an optical bio-disc 110 according to the present invention as implemented to conduct the cell counts and differential cell counts disclosed herein. The present optical bio-disc 110 is shown in conjunction with an optical disc drive 112 and a display monitor 114. Further details relating to this type of disc drive and disc analysis system are disclosed in commonly assigned and co-pending U.S. patent application Ser. No. 10/008,156 entitled “Disc Drive System and Methods for Use with Bio-discs” filed Nov. 9, 2001 and U.S. patent application Ser. No. 10/043,688 entitled “Optical Disc Analysis System Including Related Methods For Biological and Medical Imaging” filed Jan. 10, 2002, both of which are herein incorporated by reference.

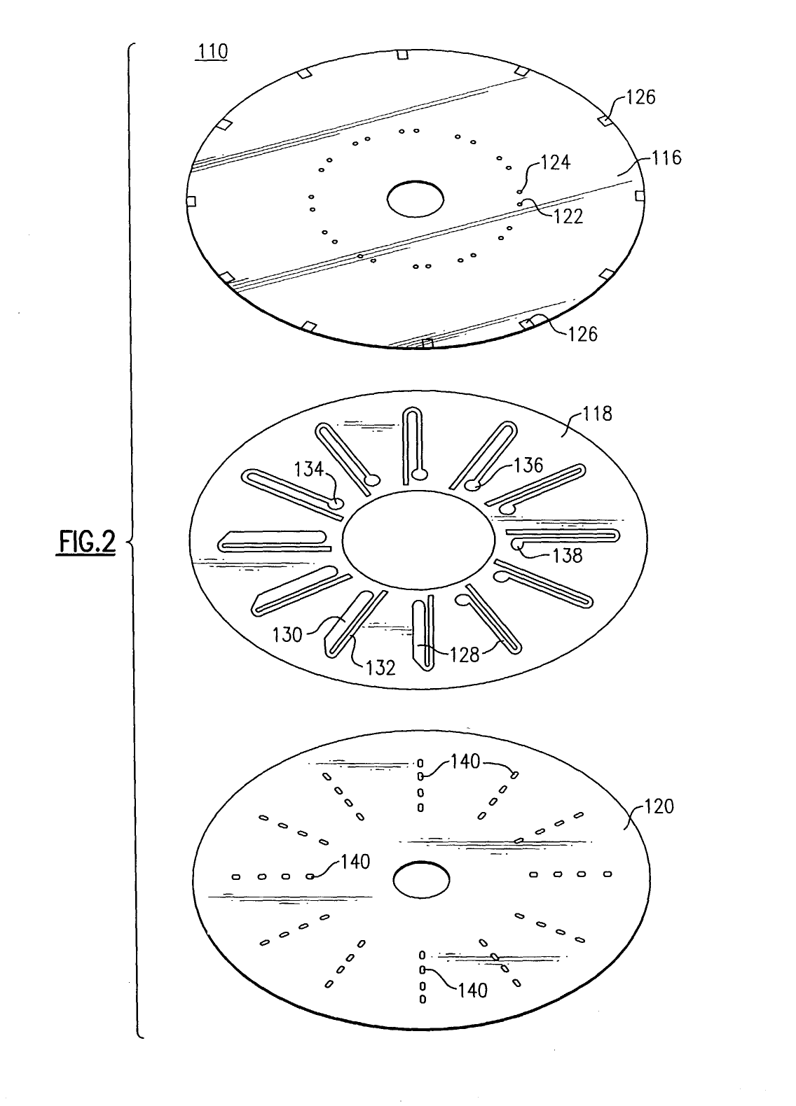

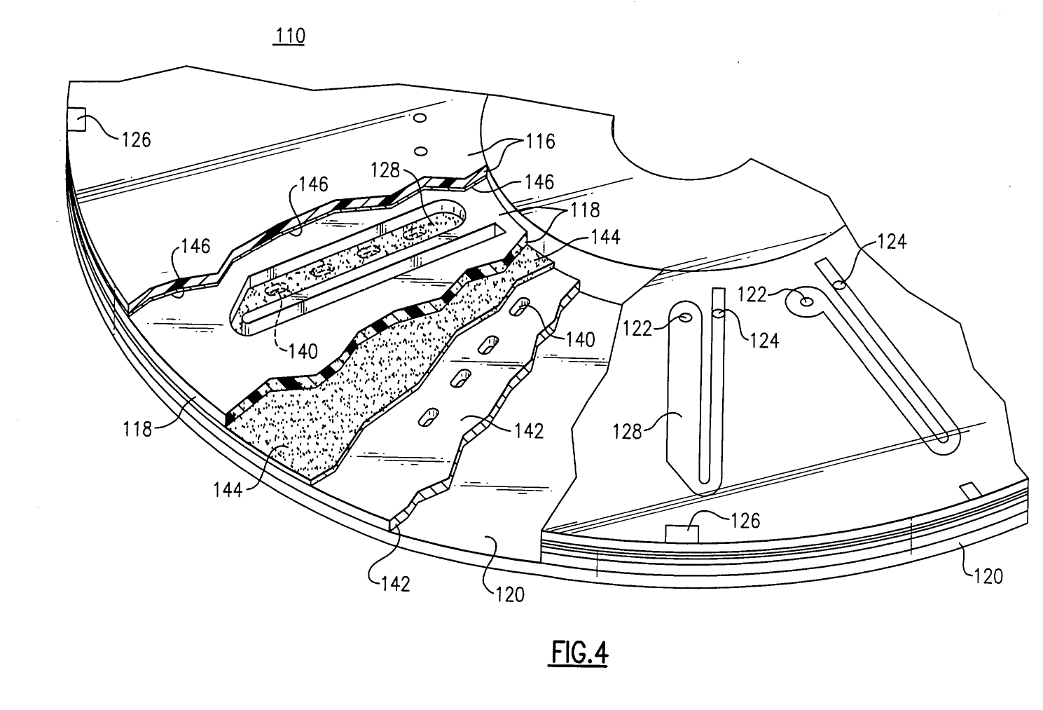

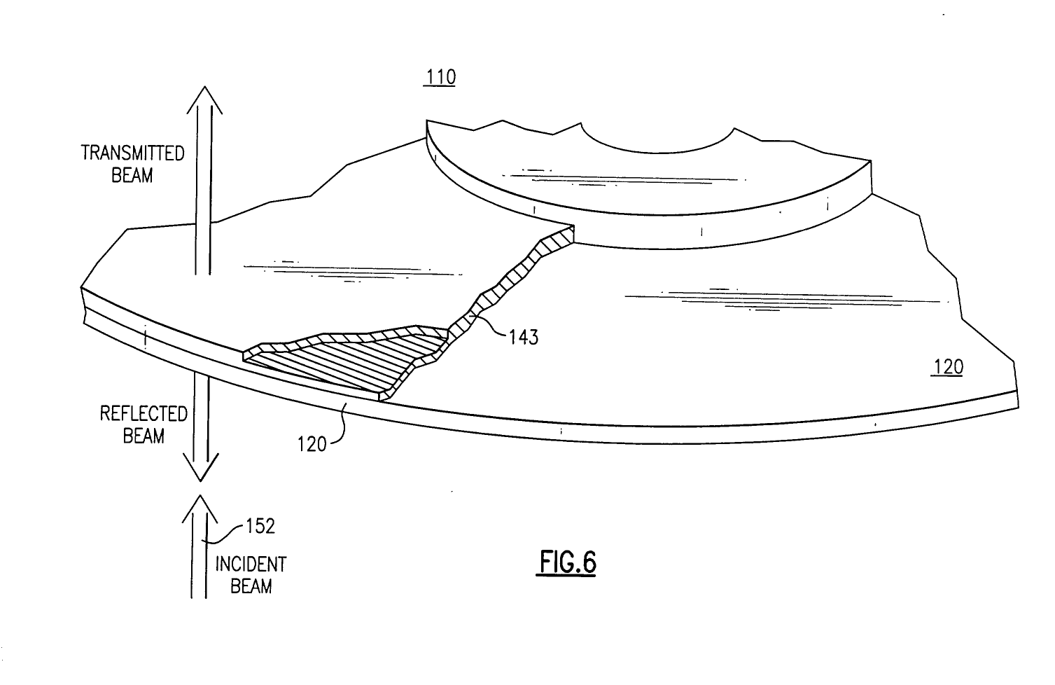

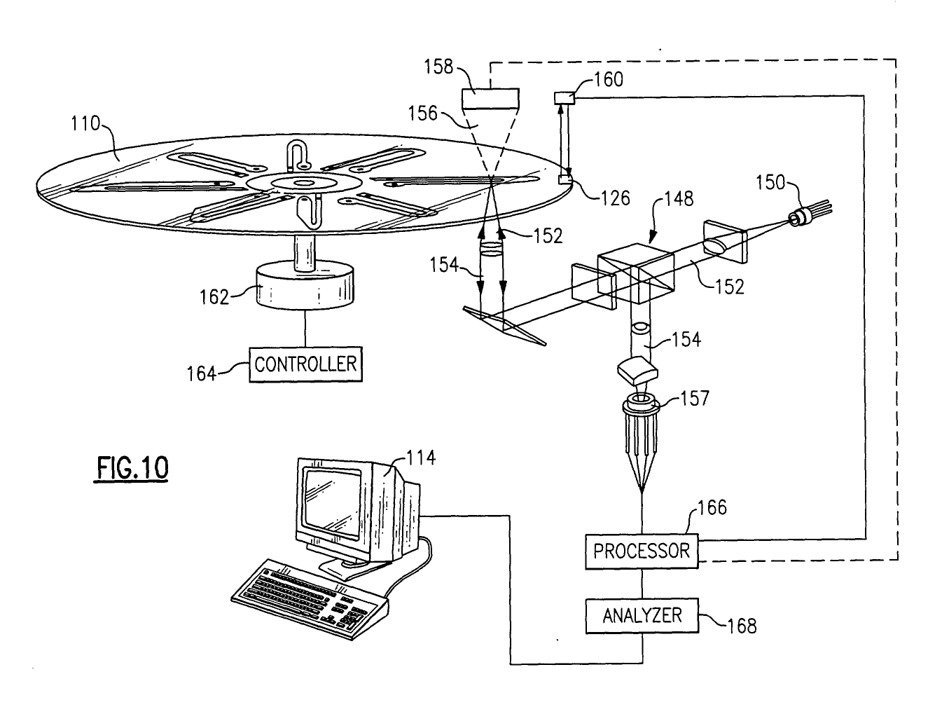

FIG. 2 is an exploded perspective view of the principal structural elements of one embodiment of the optical bio-disc 110. FIG. 2 is an example of a reflective zone optical bio-disc 110 (hereinafter “reflective disc”) that may be used in the present invention. The principal structural elements include a cap portion 116, an adhesive member or channel layer 118, and a substrate 120. The cap portion 116 includes one or more inlet ports 122 and one or more vent ports 124. The cap portion 116 may be formed from polycarbonate and is preferably coated with a reflective surface 146 (FIG. 4) on the bottom thereof as viewed from the perspective of FIG. 2. In the preferred embodiment, trigger marks or markings 126 are included on the surface of the reflective layer 142 (FIG. 4). Trigger markings 126 may include a clear window in all three layers of the bio-disc, an opaque area, or a reflective or semi-reflective area encoded with information that sends data to a processor 166, as shown FIG. 10, that in turn interacts with the operative functions of the interrogation or incident beam 152, FIGS. 6 and 10.

The second element shown in FIG. 2 is an adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes a flow channel 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

The third element illustrated in FIG. 2 is a substrate 120 including target or capture zones 140. The substrate 120 is preferably made of polycarbonate and has a reflective layer 142 deposited on the top thereof, FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

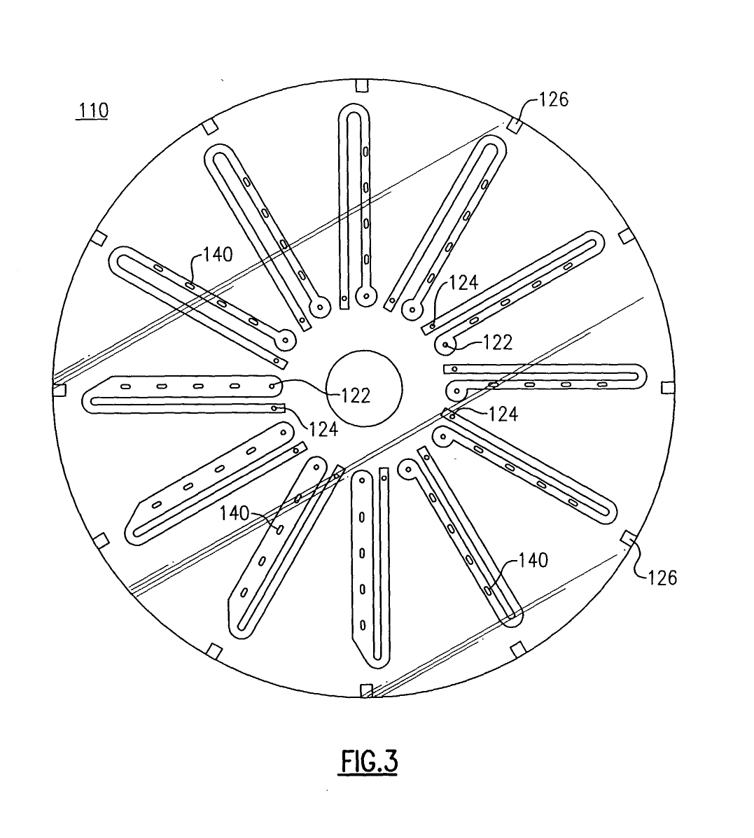

FIG. 3 is a top plan view of the optical bio-disc 110 illustrated in FIG. 2 with the reflective layer 142 on the cap portion 116 shown as transparent to reveal the fluidic circuits 128, the target zones 140, and trigger markings 126 situated within the disc.

FIG. 4 is an enlarged perspective view of the reflective zone type optical bio-disc 110 according to one embodiment of the present invention. This view includes a portion of the various layers thereof, cut away to illustrate a partial sectional view of each principal layer, substrate, coating, or membrane. FIG. 4 shows the substrate 120 that is coated with the reflective layer 142. An active layer 144 is applied over the reflective layer 142. In the preferred embodiment, the active layer 144 may be formed from polystyrene. Alternatively, polycarbonate, gold, activated glass, modified glass, or modified polystyrene, for example, polystyrene-co-maleic anhydride, may be used. In addition, hydrogels can be used. Alternatively as illustrated in this embodiment, the plastic adhesive member 118 is applied over the active layer 144. The exposed section of the plastic adhesive member 118 illustrates the cut out or stamped U-shaped form that creates the fluidic circuits 128. The final principal structural layer in this reflective zone embodiment of the present bio-disc is the cap portion 116. The cap portion 116 includes the reflective surface 146 on the bottom thereof. The reflective surface 146 may be made from a metal such as aluminum or gold.

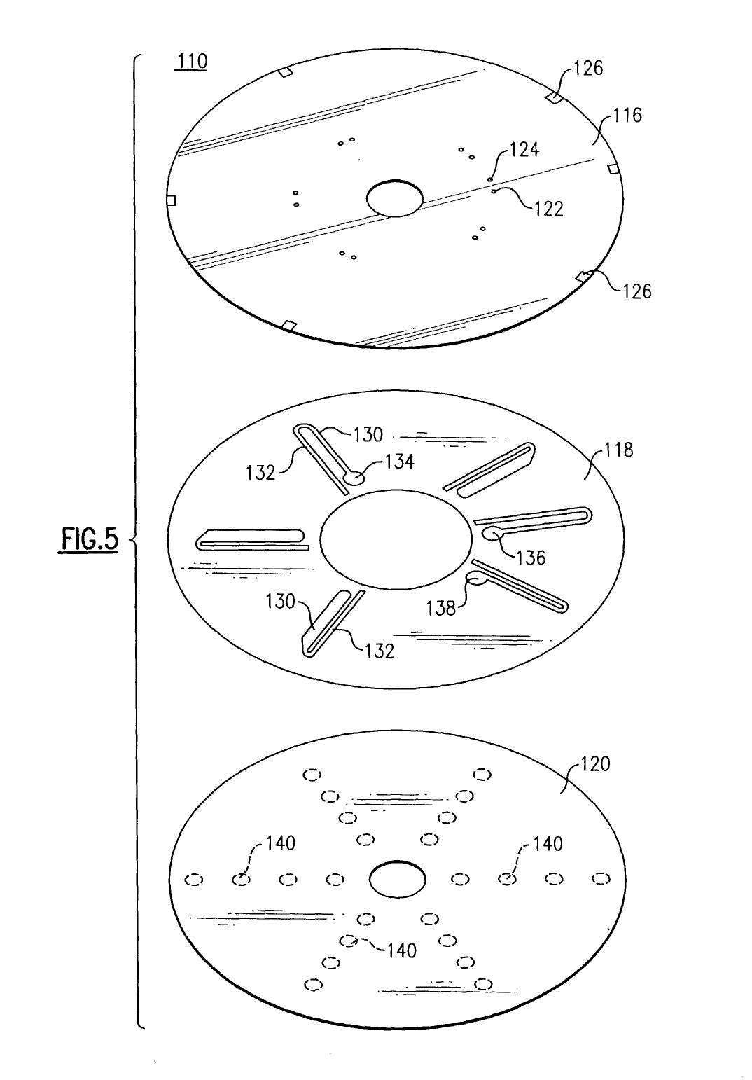

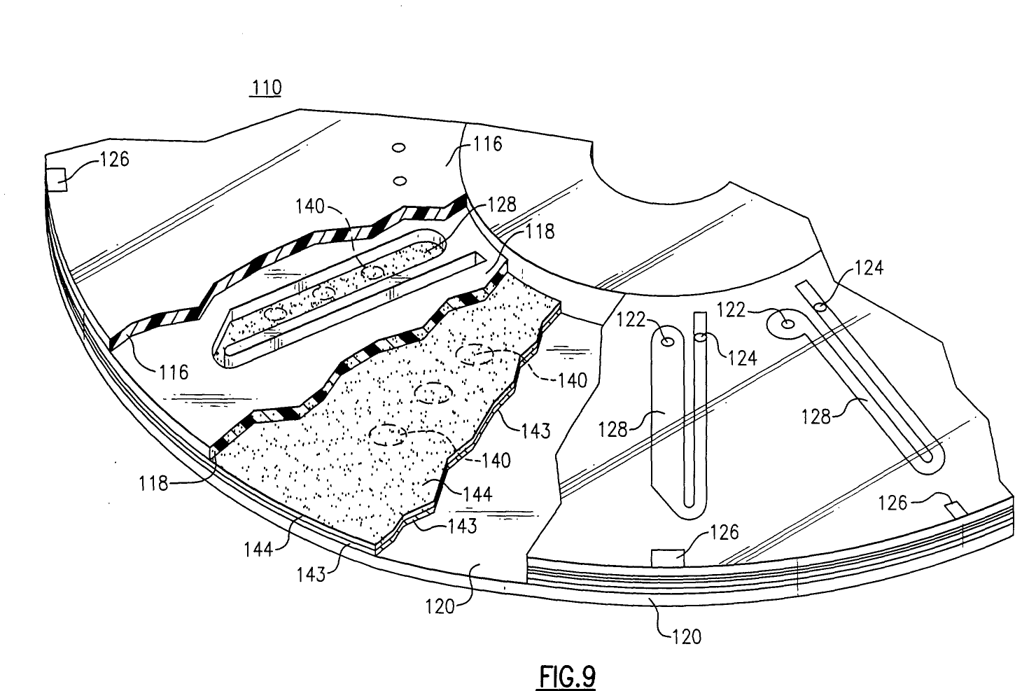

Referring now to FIG. 5, there is shown an exploded perspective view of the principal structural elements of a transmissive type of optical bio-disc 110 according to the present invention. The principal structural elements of the transmissive type of optical bio-disc 110 similarly include the cap portion 116, the adhesive or channel member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as best illustrated in FIGS. 6 and 9. Trigger markings 126 may include a clear window in all three layers of the bio-disc, an opaque area, or a reflective or semi-reflective area encoded with information that sends data to the processor 166, FIG. 10, which in turn interacts with the operative functions of the interrogation beam 152, FIGS. 6 and 10.

The second element shown in FIG. 5 is the adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include the mixing chamber 134. Two different types of mixing chambers 134 are illustrated. The first is the symmetric mixing chamber 136 that is symmetrically formed relative to the flow channel 130. The second is the off-set mixing chamber 138. The off-set mixing chamber 138 is formed to one side of the flow channel 130 as indicated.

The third element illustrated in FIG. 5 is the substrate 120 which may include the target or capture zones 140. The substrate 120 is preferably made of polycarbonate and has the thin semi-reflective layer 143 deposited on the top thereof, FIG. 6. The semi-reflective layer 143 associated with the substrate 120 of the disc 110 illustrated in FIGS. 5 and 6 is significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIGS. 6 and 12. The thin semi-reflective layer 143 may be formed from a metal such as aluminum or gold.

FIG. 6 is an enlarged perspective view of the substrate 120 and semi-reflective layer 143 of the transmissive embodiment of the optical bio-disc 110 illustrated in FIG. 5. The thin semi-reflective layer 143 may be made from a metal such as aluminum or gold. In the preferred embodiment, the thin semi-reflective layer 143 of the transmissive disc illustrated in FIGS. 5 and 6 is approximately 100-300 Å thick and does not exceed 400 Å. This thinner semi-reflective layer 143 allows a portion of the incident or interrogation beam 152 to penetrate and pass through the semi-reflective layer 143 to be detected by a top detector 158, FIGS. 10 and 12, while some of the light is reflected or returned back along the incident path. As indicated below, Table 2 presents the reflective and transmissive characteristics of a gold film relative to the thickness of the film. The gold film layer is fully reflective at a thickness greater than 800 Å. While the threshold density for transmission of light through the gold film is approximately 400 Å.

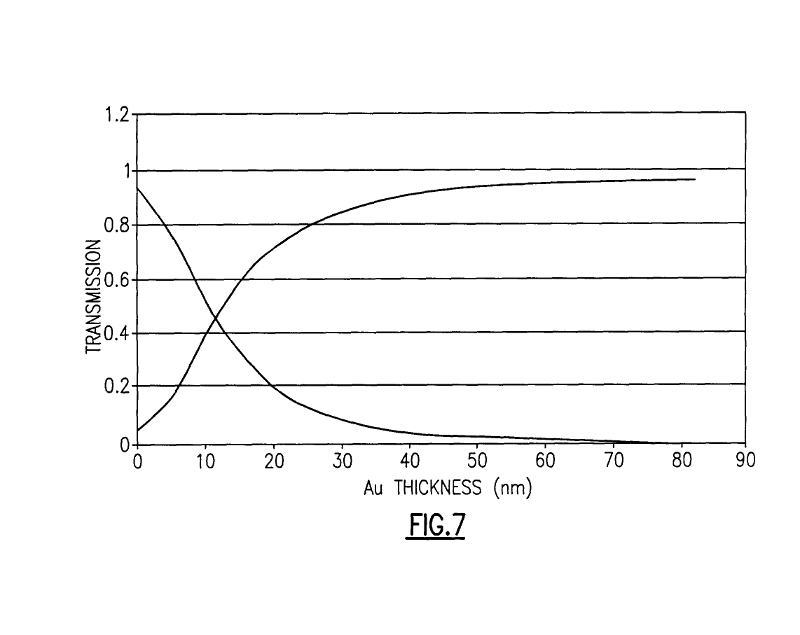

In addition to Table 2, FIG. 7 provides a graphical representation of the inverse relationship of the reflective and transmissive nature of the thin semi-reflective layer 143 based upon the thickness of the gold. Reflective and transmissive values used in the graph illustrated in FIG. 7 are absolute values.

TABLE 2
Au film Reflection and Transmission (Absolute Values)

| Thickness | | | |
|---|---|---|---|
| (Angstroms) | Thickness (nm) | Reflectance | Transmittance |
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.055 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9448 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

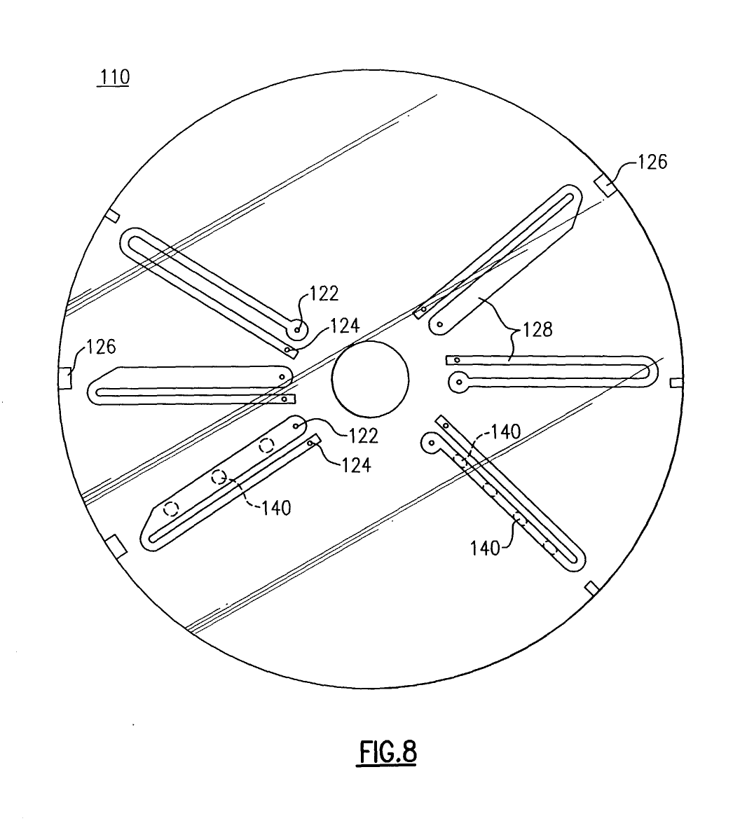

With reference next to FIG. 8, there is shown a top plan view of the transmissive type optical bio-disc 110 illustrated in FIGS. 5 and 6 with the transparent cap portion 116 revealing the fluidic channels, the trigger markings 126, and the target zones 140 as situated within the disc.

FIG. 9 is an enlarged perspective view of the optical bio-disc 110 according to the transmissive disc embodiment of the present invention. The disc 110 is illustrated with a portion of the various layers thereof cut away to show a partial sectional view of each principal layer, substrate, coating, or membrane. FIG. 9 illustrates a transmissive disc format with the clear cap portion 116, the thin semi-reflective layer 143 on the substrate 120, and trigger markings 126. In this embodiment, trigger markings 126 include opaque material placed on the top portion of the cap. Alternatively the trigger marking 126 may be formed by clear, non-reflective windows etched on the thin reflective layer 143 of the disc, or any mark that absorbs or does not reflect the signal coming from the trigger detector 160, FIG. 10. FIG. 9 also shows, the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking the entire thin semi-reflective layer 143 except the target zones 140. In this embodiment, target zones 140 may be created by silk screening ink onto the thin semi-reflective layer 143. In the transmissive disc format illustrated in FIGS. 5, 8, and 9, the target zones 140 may alternatively be defined by address information encoded on the disc. In this embodiment, target zones 140 do not include a physically discernable edge boundary.

With continuing reference to FIG. 9, an active layer 144 is illustrated as applied over the thin semi-reflective layer 143. In the preferred embodiment, the active layer 144 is a 10 to 200 μm thick layer of 2% polystyrene. Alternatively, polycarbonate, gold, activated glass, modified glass, or modified polystyrene, for example, polystyrene-co-maleic anhydride, may be used. In addition, hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128.

The final principal structural layer in this transmissive embodiment of the present bio-disc 110 is the clear, non-reflective cap portion 116 that includes inlet ports 122 and vent ports 124.

Referring now to FIG. 10, there is a representation in perspective and block diagram illustrating optical components 148, a light source 150 that produces the incident or interrogation beam 152, a return beam 154, and a transmitted beam 156. In the case of the reflective bio-disc illustrated in FIG. 4, the return beam 154 is reflected from the reflective surface 146 of the cap portion 116 of the optical bio-disc 110. In this reflective embodiment of the present optical bio-disc 110, the return beam 154 is detected and analyzed for the presence of signal elements by a bottom detector 157. In the transmissive bio-disc format, on the other hand, the transmitted beam 156 is detected, by a top detector 158, and is also analyzed for the presence of signal elements. In the transmissive embodiment, a photo detector may be used as a top detector 158.

FIG. 10 also shows a hardware trigger mechanism that includes the trigger markings 126 on the disc and a trigger detector 160. The hardware triggering mechanism is used in both reflective bio-discs (FIG. 4) and transmissive bio-discs (FIG. 9). The triggering mechanism allows the processor 166 to collect data only when the interrogation beam 152 is on a respective target zone 140. Furthermore, in the transmissive bio-disc system, a software trigger may also be used. The software trigger uses the bottom detector to signal the processor 166 to collect data as soon as the interrogation beam 152 hits the edge of a respective target zone 140. FIG. 10 further illustrates a drive motor 162 and a controller 164 for controlling the rotation of the optical bio-disc 110. FIG. 10 also shows the processor 166 and analyzer 168 implemented in the alternative for processing the return beam 154 and transmitted beam 156 associated the transmissive optical bio-disc.

As shown in FIG. 11, there is presented a partial cross sectional view of the reflective disc embodiment of the optical bio-disc 110 according to the present invention. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As also shown in FIG. 11, the target zone 140 is formed by removing an area or portion of the reflective layer 142 at a desired location or, alternatively, by masking the desired area prior to applying the reflective layer 142. As further illustrated in FIG. 11, the plastic adhesive member 118 is applied over the active layer 144. FIG. 11 also shows the cap portion 116 and the reflective surface 146 associated therewith. Thus when the cap portion 116 is applied to the plastic adhesive member 118 including the desired cutout shapes, flow channel 130 is thereby formed. As indicated by the arrowheads shown in FIG. 11, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return beam 154.

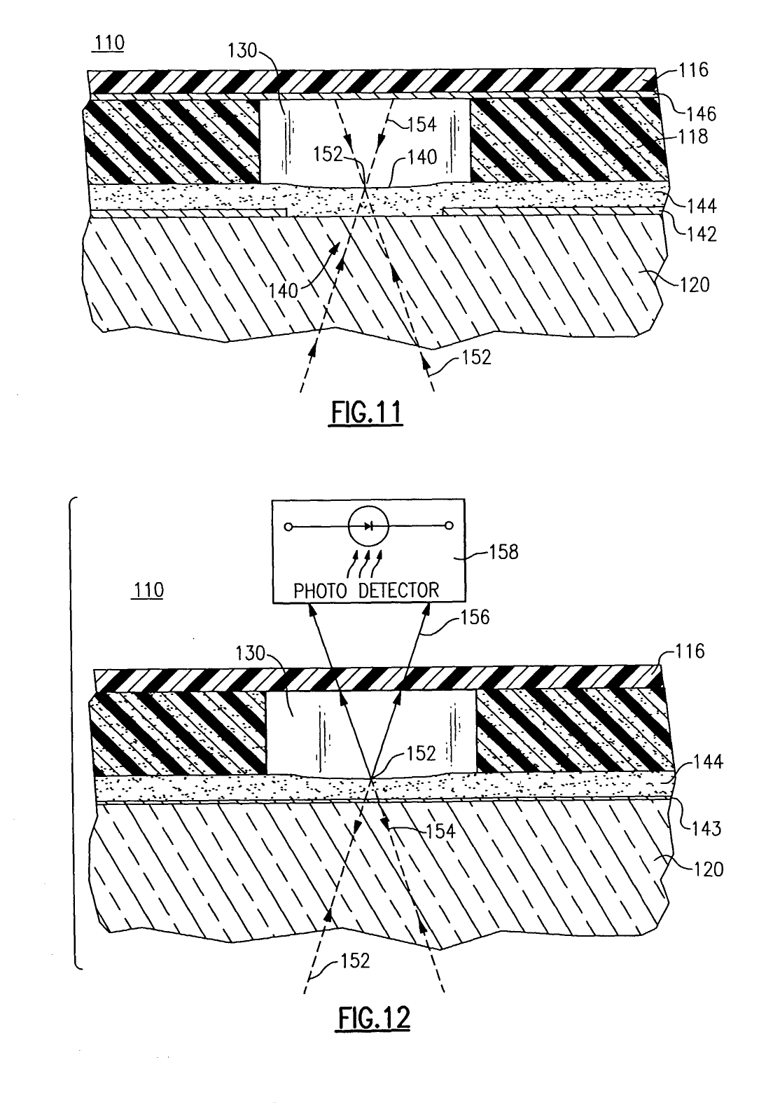

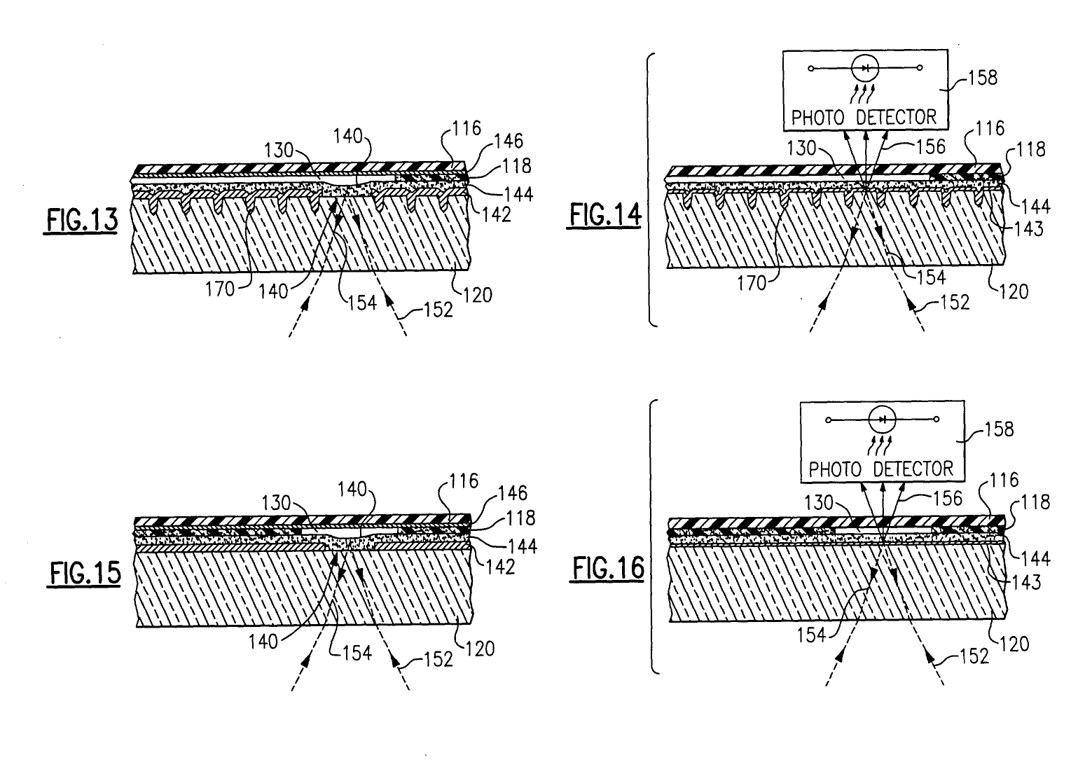

FIG. 12 is a partial cross sectional view of the transmissive embodiment of the bio-disc 110 according to the present invention. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100-300 Angstroms thick and does not exceed 400 Angstroms. This thin semi-reflective layer 143 allows a portion of the incident or interrogation beam 152, from the light source 150, FIG. 10, to penetrate and pass upwardly through the disc to be detected by a top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a physically defined target zone 140 may or may not be present. Target zone 140 may be created by direct markings made on the thin semi-reflective layer 143 on the substrate 120. These marking may be formed using silk screening or any equivalent method. In the alternative embodiment where no physical indicia are employed to define a target zone (such as, for example, when encoded software addressing is utilized) the flow channel 130 in effect may be employed as a confined target area in which inspection of an investigational feature is conducted.

FIG. 13 is a cross sectional view taken across the tracks of the reflective disc embodiment of the bio-disc 110 according to the present invention. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove”. A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 according to the present invention as described in FIG. 12, for example. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light required by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive member or channel layer 118 is applied over the active layer 144. FIG. 14 also shows the cap portion 116 without a reflective surface 146. Thus, when the cap is applied to the plastic adhesive member 118 including the desired cutout shapes, the flow channel 130 is thereby formed and a part of the incident beam 152 is allowed to pass therethrough substantially unreflected.

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these figures, the incident beam 152 is illustrated initially interacting with the substrate 120 which has refractive properties that change the path of the incident beam as illustrated to provide focusing of the beam 152 on the reflective layer 142 or the thin semi-reflective layer 143.

Cellular Assays

A generic homogeneous solid phase cell capture assay for the rapid determination of absolute number of CD4⁺ and CD8⁺ T-lymphocyte populations and ratio of CD4⁺/CD8⁺ lymphocytes in blood samples may be performed utilizing the methods of the invention. The test, which is run within small flow channels incorporated into a bio-disc, determines the number of CD4⁺, CD8⁺, CD2⁺, CD3⁺, CD19⁺, and CD45⁺ cells captured by the specific antibodies on the capture zones in 7-15 μl of mononuclear cells (MNC) isolated from whole blood. The test is based upon the principle of localized cell capture on specific locations on the disc. Several specific cell capture zones are created on the disc by localized application of capture chemistries based upon monoclonal or polyclonal antibodies to particular blood cell surface antigens. Upon flooding the 25-100 μl chambers with the MNC blood (10,000-30,000 cells/μl), cells expressing CD4, CD8, CD2, CD3, CD19, and CD45 antigens are captured in the capture zones within the disc. Also incorporated within the capture zones are defined negative control areas.

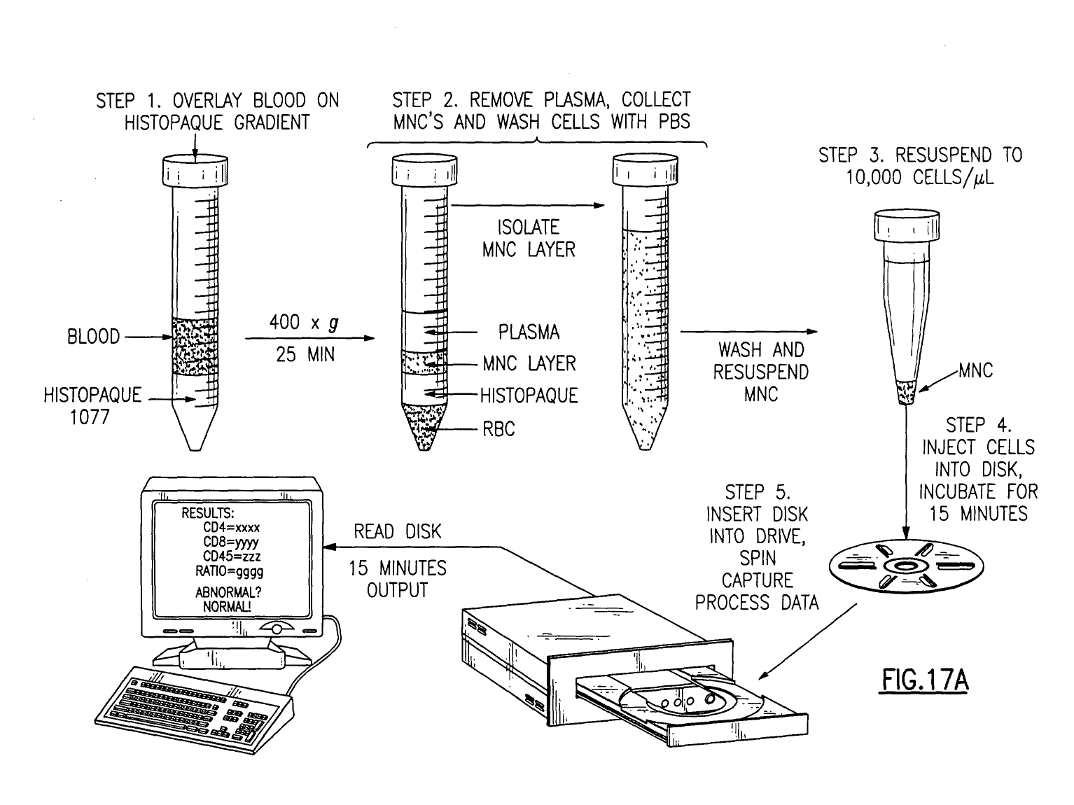

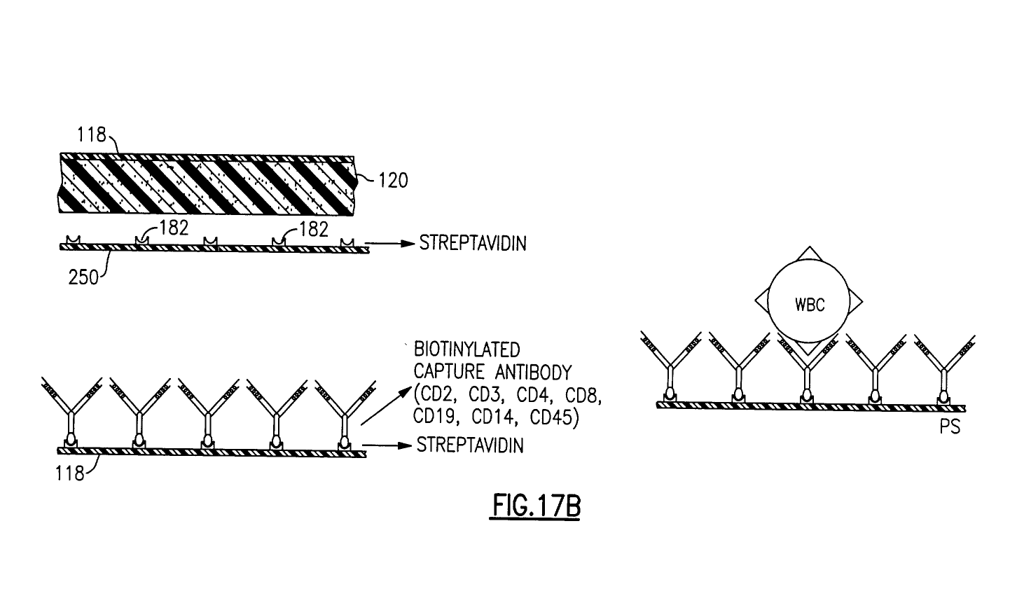

FIG. 17A is a pictorial flow chart showing the analysis of a blood sample. In step 1, blood (4-8 ml) is collected directly into a 4 or 8 ml Becton Dickinson CPT Vacutainer™ and an anticoagulant such as EDTA, acid citrate dextrose (ACD), or heparin. In an equivalent step of another embodiment of the invention, 3 ml of blood in anticoagulant is overlayed into a tube 172 containing a separation gradient 176 such as Histopaque® 1077. In any case, the blood sample 174 is preferably used within two hours of collection. The tube 172 containing the separation gradient 176 with blood sample 174 overlay is centrifuged at 400×g in a biohazard centrifuge with horizontal rotor and swing out buckets for 30 minutes at room temperature. After centrifugation, the plasma layer 178 is removed (step 2), leaving about 2 mm of plasma above the mononuclear cell (MNC) fraction 180. The MNC layer 180 is collected and washed with phosphate buffer saline (PBS). Cells are pelleted by centrifugation at 250×g for 10 minutes at room temperature to remove any remaining platelets. The supernatant is removed and the MNC pellet 180 is re-suspended in PBS by tapping the tube gently. The final pellet 180 is re-suspended (step 3) to a cell count of 10,000-30,000 cells/μl depending upon the height of the flow channel 130 of the bio-disc 110.

The flow channel 130 of a bio-disc 110 is flooded with 7 μl of the MNC suspension, and the inlet ports 122 and vent ports 124 (FIGS. 3 and 8) of the chamber are sealed with an adhesive tab or other suitable sealing member (step 4). The bio-disc 110 is incubated for 15 minutes at room temperature, and then scanned using a 780 nm laser in an optical drive 112 to image the capture field (step 5). It should be understood that if a transmissive bio-disc 110 is used, optical drive 112 optionally includes a top detector 158 (FIG. 10) to image the capture field. Software is preferably encoded on the disc to instruct the drive to automatically perform the following tasks: (a) centrifuge the disc to spin off excess unbound cells in one or more stages; (b) image specific capture windows on a display monitor 114; and (c) process data. Data processing includes, but is not limited to, counting the specifically captured cells in each capture zone and deriving the ratio of CD4⁺/CD8⁺ or whichever ratio is programmed to be determined. Other desired ratios may be readily provided by alternative embodiments of the present invention.

As further illustrated in FIG. 17A, the present invention is directed to a method of performing a cluster designation count with an optical disc and disc drive system. The method includes the steps of providing a blood sample in a first tube containing a separation gradient, rotating the first tube at a time and speed sufficient to separate the blood sample into layers, resuspending a MNC layer that contains T-cells to form a MNC suspension, providing a sample of the MNC suspension on a disc surface that includes at least one capture zone containing at least one capture agent, loading the disc into an optical reader, rotating the disc, directing an incident beam of electromagnetic radiation to the capture zone, detecting a beam of electromagnetic radiation formed after interacting with the disc at the capture zone, converting the detected beam into an output signal, and analyzing the output signal to extract information relating to the number of cells captured at the capture zone. In one embodiment of this method, the optical disc is constructed with a reflective layer such that light directed to the capture zone and not striking a cell is reflected. In another embodiment of this method, the optical disc is constructed such that light directed to the capture zone and not striking a cell is transmitted through the optical disc.

During the analyzing/processing step, the software reads across each capture zone image and marks cell images as it encounters them. For example, following counting of the number of captured CD4⁺ and CD8⁺ cells, the software calculates the ratio of CD4⁺/CD8⁺ cells and displays both the absolute numbers of cells in CD4⁺, CD8⁺, CD3⁺ and CD45⁺ capture zones per microliter of whole blood and also the CD4⁺/CD8⁺ ratio. The entire process takes about 12 minutes from inserting the disc into the optical drive to obtaining the numbers and ratios.

In one embodiment, the disc is a forward Wobble Set FDL21 :13707 or FDL21:1270 CD-R disc coated with 300 nm of gold as the encoded information layer. On a reflective disc, viewing windows of size 2×1 mm oval are etched out of the reflective layer by known lithography techniques. In some designs of transmissive disc, no separate viewing windows are etched, and the entire disc is available for use. In one specific embodiment, the adhesive layer is Fraylock adhesive DBL 201 Rev C 3M94661. The cover is a clear disc with 48 sample inlets with a diameter of 0.040 inches located equidistantly at radius 26 mm. The data disc is scanned and read with the software at speed 4× and sample rate 2.67 MHz using CD4⁺/CD8⁺ counting software.

Referring to FIG. 17B, in one embodiment of the present invention, a thick layer of polystyrene 118 is formed over a substrate 120 and is (optionally) layered with streptavidin 182. Cell capture antibodies are attached to the strepavidin 182 through biotin. These antibodies can include biotinylated antibodies attached to Dextran-activated aldehyde coated over the streptavidin to create an ample number of binding sites for the capture antibody. This creates a strong capture chemistry that can specifically form robust bonds with white blood cells (WBCs).

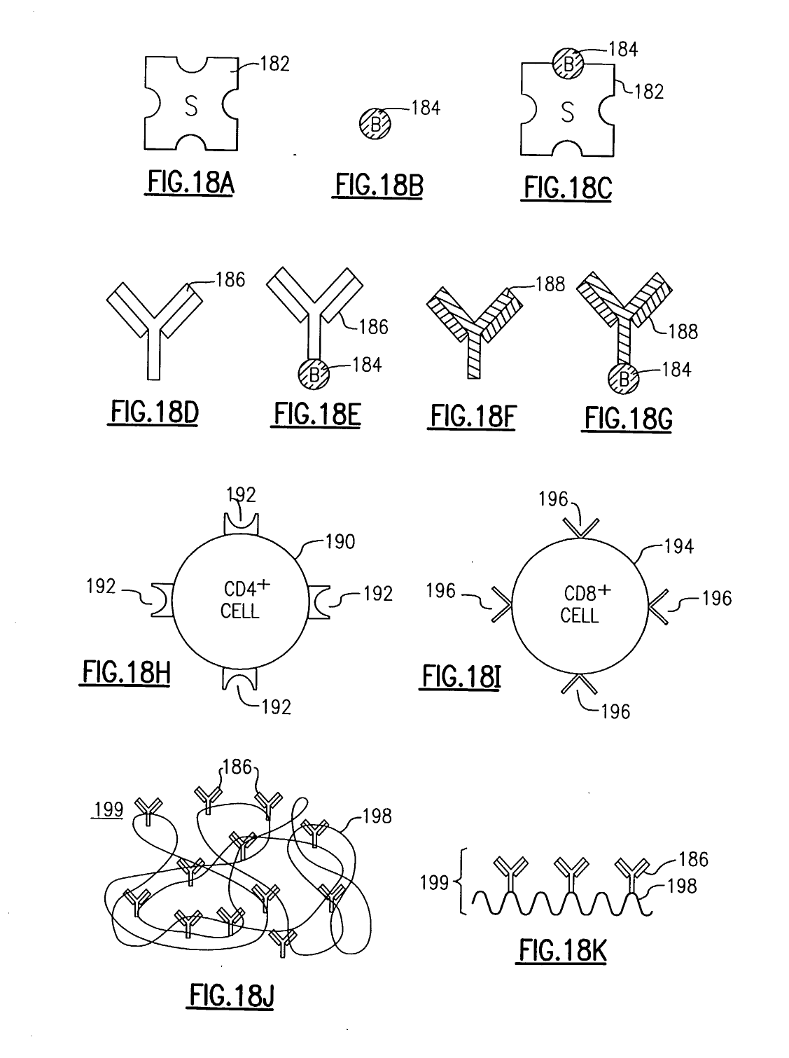

FIGS. 18A, 18B, and 18C are pictorial representations of a cross-linking system used in an embodiment of the present invention. It should be understood that a cross-linking system involves one or more cross-linking agents, or conjugates, to cross-link one or more macromolecular moieties to another. A cross-link may be a covalent or non-covalent interaction between two macromolecular moieties, usually formed when two macromolecular free radicals combine. Chemical modifications or conjugation processes to achieve cross-links involve the reaction of one functional group with another, resulting in the formation of a bond. The creation of bioconjugate reagents with reactive or selectively reactive functional groups forms the basis for simple and reproducible cross-linking or tagging of target molecules (“Bioconjugate Techniques,” Greg T. Hermanson, Academic Press, San Diego, Calif., (1996)).

Cross-linking agents include, but are not limited to homobifunctional linkers, heterobifunctional linkers, and zero-length cross-linkers. Homobifunctional linkers are linkers with two reactive sites of the same functionality, such as glutaraldehyde. These reagents could tie one protein to another by covalently reacting with the same common groups on both molecules. Heterobifunctional conjugation reagents contain two different reactive groups that can couple to two different functional targets on proteins and other macromolecules. For example, one part of a cross-linker may contain an amine-reactive group, while another portion may consist of a sulfhydryl-reactive group. The result is the ability to direct the cross-linking reaction to selected parts of target molecules, thus garnering better control over the conjugation process. Zero-length cross-linkers mediate the conjugation of two molecules by forming a bond containing no additional atoms. Thus, one atom of a molecule is covalently attached to an atom of a second molecule with no intervening linker or spacer. One of ordinary skill in the art may refer to “Bioconjugate Techniques,” Greg T. Hermanson, Academic Press, San Diego, Calif., (1996), for a detailed description of cross-linking agents.

In the present invention, cross-linking agents are bound to the surface of a bio-disc to immobilize capture agents within the target zones. A preferred cross-linking system is the heterobifunctional group consisting of biotin-streptavidin, i.e. biotinylated capture agents bound to an avidin-coupled substrate. FIG. 18A is a pictorial representation of streptavidin 182. Without limitation, streptavidin includes avidin, streptavidin, and modifications, thereof. As shown, the protein comprises four subunits, each of which contains one binding site for biotin (Hermanson). Streptavidin 182 can be coupled to plastics such as polystyrene by various chemistries. Ideally, streptavidin 182 is attached to the active layer 144 (FIGS. 4 and 9) of the bio-disc, binding essentially irreversibly to biotinylated sensing elements (e.g. antibodies).

FIG. 18B is a pictorial representation of biotin 184. Biotin (or vitamin H) is a naturally occurring growth factor present in small amounts within every cell. Biotin's interaction with the proteins avidin and streptavidin is among the strongest non-covalent affinities known. A biotin molecule 184 may be attached directly to a protein via its valeric acid side chain or derivitized with other organic components to create spacer arms and various reactive groups. Amines, carboxylates, sulfhydryls, and carbohydrate groups can be specifically targeted for biotinylation through the appropriate choice of biotin derivative (Hermanson). FIG. 18C is a pictorial representation of the cross-linking system consisting of biotin 184 interacting with streptavidin 182.

Implementations of the embodiments of the invention utilize capture agents to perform the assays described herein. It should be understood that a capture agent refers to any macromolecule for detecting an analyte. The capture agents of the invention include macromolecules preferentially selective, or having a selective binding affinity, for an analyte of interest. Capture agents include, but are not limited to, synthetic or biologically produced nucleic acid and synthetic or biologically produced proteins. Examples of capture agents that can be employed by this invention, include, but are not restricted to, deoxyribonucleic acid (DNA), ribonucleic acid (RNA), oligonucleotides, polymerase chain reaction products, or a combination of these nucleotides (chimera), antibodies (monoclonal or polyclonal), cell membrane receptors, and anti-sera reactive with specific antigenic determinants (such as on viruses, cells, or other materials), drugs, peptides, co-factors, lectins, polysaccharides, cells, cellular membranes, and organelles. Preferably, capture agents of the invention are antibodies.

Antibodies include, but are not limited to polyclonal, monoclonal, and recombinantly created antibodies. Antibodies of the invention can be produced in vivo or in vitro. Methods for the production of antibodies are well known to those skilled in the art. For example, see Antibody Production: Essential Techniques, Peter Delves (Ed.), John Wiley & Son Ltd, ISBN: 0471970107 (1997), which is incorporated herein in its entirely by reference. Alternatively, antibodies may be obtained from commercial sources, e.g., Research Diagnostics Inc., Pleasant Hill Road, Flanders, N.J. 07836. Antibodies of the invention are not meant to be limited to antibodies of any one particular species; for example, antibodies of humans, mice, rats, and goats are all contemplated by the invention. Preferably, the primary antibodies of the invention are anti-human produced in mice, and the secondary antibodies of the invention are anti-mouse produced in goats.

The term “antibody” is also inclusive of any class or subclass of antibodies, as any or all antibody types may be used to bind to cell surface antigens. The use of antibodies in the art of medical diagnostics is well known to those skilled in the art. For example, see Diagnostic and Therapeutic Antibodies (Methods in Molecular Medicine), Andrew J. T. George and Catherine E. Urch (Eds.), Humana Press; ISBN: 0896037983 (2000) and Antibodies in Diagnosis and Therapy: Technologies, Mechanisms and Clinical Data (Studies in Chemistry Series), Siegfried Matzku and Rolf A. Stahel (Eds.), Harwood Academic Pub.; ISBN: 9057023105 (1999), which are incorporated entirely herein by reference.

In at least some embodiments of the invention, a plurality of capture agents is used to detect analytes of interest. FIG. 18D is a pictorial representation of the IgG class of antibodies used in the methods of the invention as a secondary capture agent 186. It should be understood that secondary capture agents of the invention include, but are not limited to, agents having an affinity for another capture agents, which have an affinity for the analyte of interest. FIG. 18E shows the secondary capture agent IgG 186 bound to a biotin molecule 184 hereinafter referred to as biotinylated-IgG.

FIG. 18F is a pictorial representation of a primary capture agent 188. It should be understood that a primary capture agent 188 of the invention has a selective affinity for the analyte of interest. Preferably, a primary capture agent is an antibody having an affinity for leukocytes. More specifically, these antibodies are directed against lymphocytes (CD2, CD19), monocytes (CD14), eosinophils (CD15), and other cell surface markers of interest. FIG. 18G shows the primary capture agent 188 bound to a biotin molecule 184. In addition to CD4 and CD8, there are antibodies against many other cell surface antigens (e.g., CD3, CD16, CD19, CD45, CD56), which can be used to identify sub-types of lymphocytes.

FIG. 18H is a pictorial representation of a CD4⁺ T-cell 190. CD4⁺ T-cells bind to specific antigens expressed by antigen-presenting cells (APCs) such as phagocytic macrophages and dendritic cells, and release lymphokines that attract other immune system cells to the area. The result is inflammation, and the accumulation of cells and molecules that attempt to wall off and destroy the antigenic material. It should be understood that CD4⁺ T-cells have a multitude of antigens 192 over the entire cell surface. However, FIG. 18H shows the CD4⁺ T-cell 190 having four antigens 192 for illustrative purposes only.

FIG. 18I is a pictorial representation of a CD8⁺ T-cell 194. These T-cells secrete molecules that destroy the cell to which they have bound. This is important in fighting viral infections, as the CD8⁺ T-cells destroy the infected cells before they can release a fresh crop of viruses able to infect other cells. It should be understood that CD8⁺ T-cells have a multitude of antigens 196 over the entire cell surface. However, FIG. 18I shows the CD8⁺ T-cell 194 having four antigens 196 for illustrative purposes only.

FIG. 18J is a pictorial representation showing secondary antibodies 186 bound to a 3-Dimentional matrix of aldehyde-activated dextran (DCHO) 198, thereby forming a DCHO-antibody complex 199. Dextran is mainly a linear polysaccharide consisting of repeating units of D-glucose linked together by glycosidic bonds. Used extensively as a cross-linking agent, dextran is multivalent in nature, which allows molecules to be attached at numerous sites along the polymer chain (Hermanson). For illustrative purposes only, antibodies 186 bound to dextran 198 will be depicted as shown in FIG. 18K.

Aspects relating the cellular assays of the present invention are also disclosed in U.S. Provisional Application Serial No. 60/308,176 entitled “Quantitative and Qualitative Methods for Characterizing Cancerous Blood Cells Including Leukemic Blood Samples Using Optical Bio-Disc Platform” filed Jul. 27, 2001; U.S. Provisional Application Serial No. 60/312,248 entitled “Methods for Quantitative and Qualitative Characterization of Cancerous Blood Cells Including Lymphoma Blood Samples Using Optical Bio-Disc Platform” filed Aug. 15, 2001; U.S. Provisional Application Serial No. 60/313,514 entitled “Methods for Specific Cell Capture by Off-Site Incubation of Primary Antibodies with Sample and Subsequent Capture by Surface-Bound Secondary Antibodies and Optical Bio-Disc Including Same” filed Aug. 20, 2001; U.S. Provisional Application Serial No. 60/313,715 entitled “RBC Lysis Protocol Evaluations of Helper/Inducer-Suppressor/Cytotoxic T-Lymphocytes Using Whole Blood and Related Optical Bio-Disc” filed Aug. 20, 2001; U.S. Provisional Application Serial No. 60/313,536 entitled “RBC Sieving Protocol Evaluations of Helper/Inducer-Suppressor/Cytotoxic T-Lymphocytes Using Whole Blood and Related Optical Bio-Disc” filed Aug. 20, 2001; and U.S. Provisional Application Serial No. 60/315,937 entitled “Quantitative and Qualitative Methods for Cell Isolation and Typing Including Immunophenotyping” filed Aug. 30, 2001, all of which are herein incorporated by reference.

Implementation I

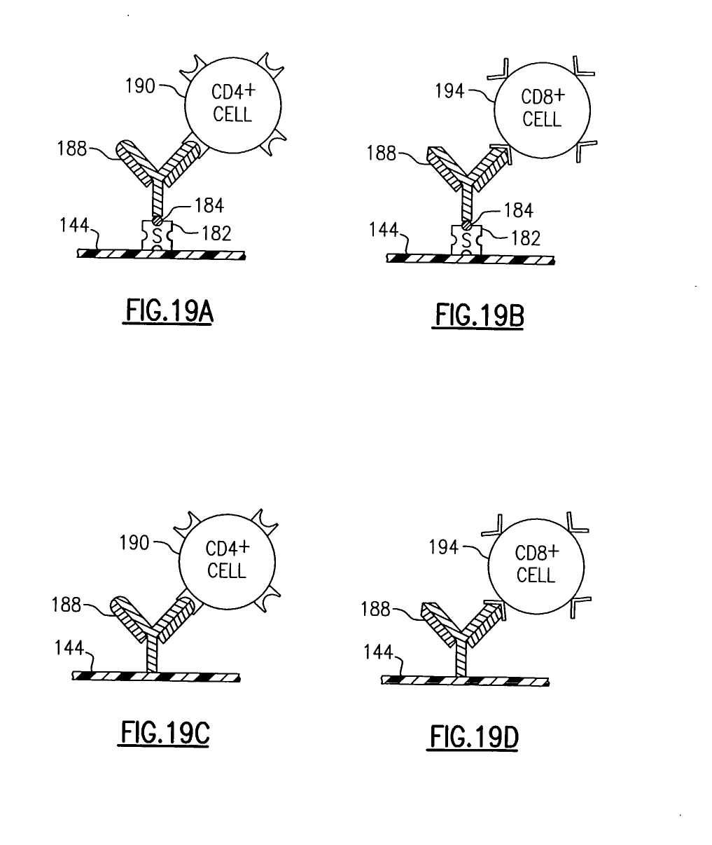

FIGS. 19A-19D are pictorial representations of analyte capture in a first implementation of the invention. FIGS. 19A and 19B show capture of CD4⁺ T-cells 190 and CD8⁺ T-cells 194 by biotinylated primary antibodies (FIG. 18G). The antibodies 188 are immobilized on the active layer 144 of the bio-disc 110 (FIGS. 4 and 9) by the cross-linking agent streptavidin 182. FIGS. 19C and 19D show another embodiment of the same implementation of the invention without a cross-linking system. In this embodiment, primary antibodies 188 are immobilized directly on the active layer 144 of the bio-disc 110.

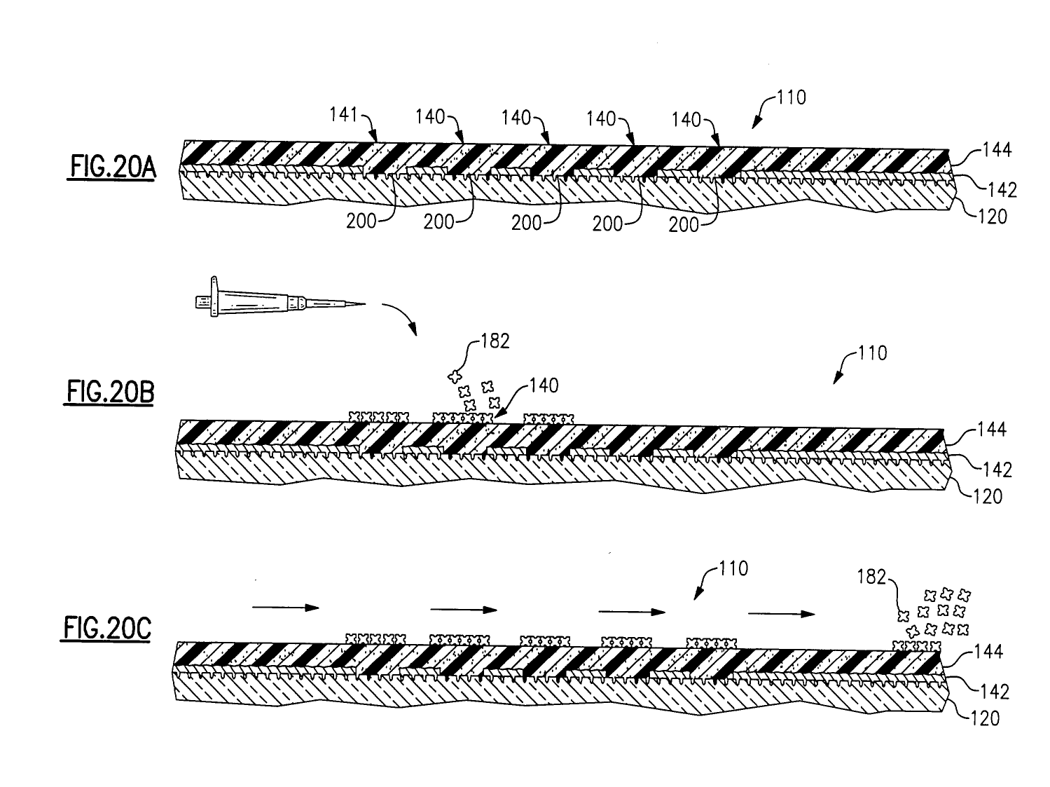

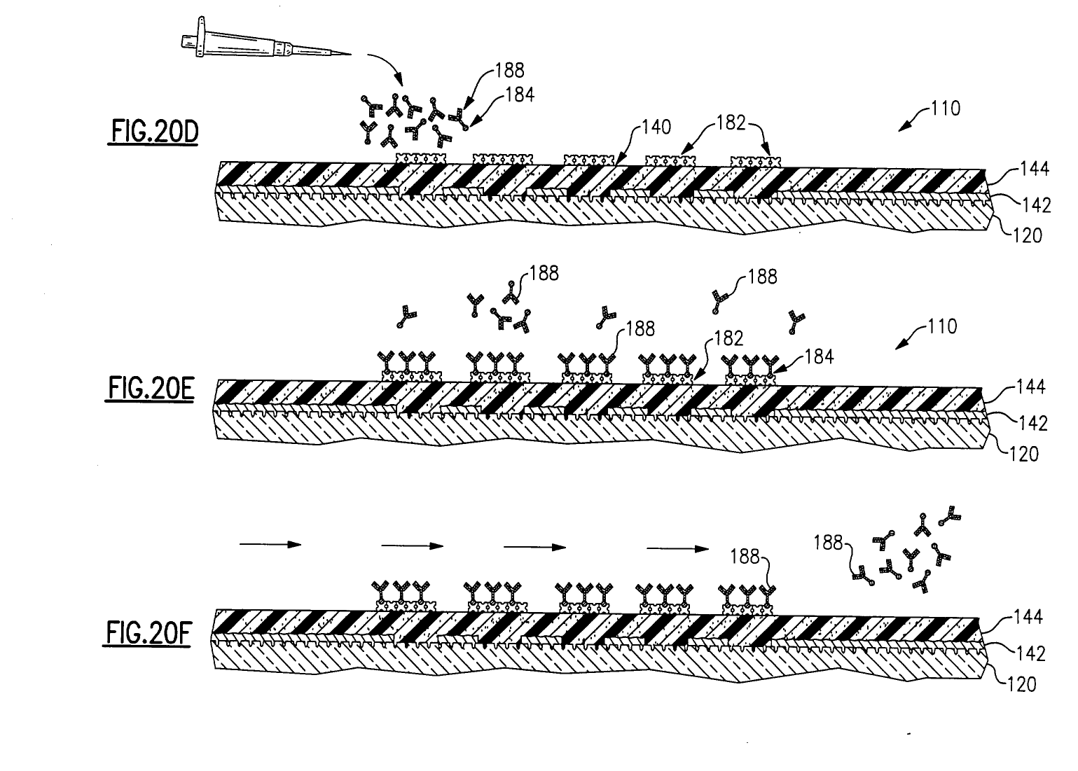

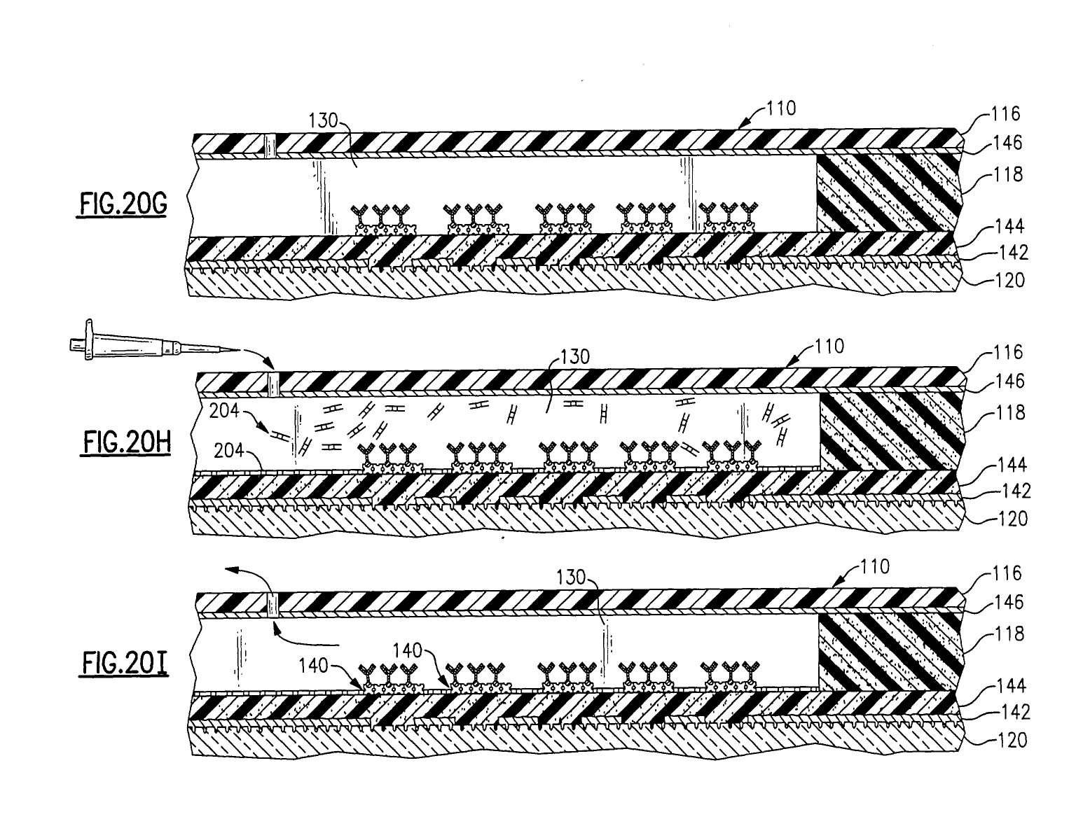

FIGS. 20A-20I are cross-sectional views showing construction of an embodiment of the first implementation of the invention. The first embodiment involves construction of a reflective disc utilizing a cross-linking system to immobilize the capture agents within the flow channels of the bio-disc. Referring to FIG. 20A, a light-transparent substrate 120, a reflective layer 142, and an active layer 144 of an optical bio-disc 110 are shown. Portions of reflective layer 142 are removed (or openings were created when deposited) to produce viewing windows 200 through which light can be directed at the locations of capture zones 140 where the antibodies are to be affixed. FIG. 20A shows five such capture zones 140, the first thereof indicated as capture zone 141. The active layer 144 is preferably polystyrene, which is spin-coated, or otherwise deposited by methods known in the art, over the reflective layer 142 to form a smooth surface with a thickness of about 40 to 300 microns. Streptavidin 182 is then deposited over each capture zone 140 and 141, and the disc 110 is incubated at room temperature in a humidity chamber for 30 minutes (FIG. 20B). The disc 110 is washed to remove unbound streptavidin 182, and then spin-dried to completely remove moisture from the surface of the disc 110 (FIG. 20C). A reference mark or calibration dot 202 is deposited over the first capture zone 141 and biotinylated primary antibodies 188 are deposited over each successive capture zone 140 (FIGS. 20D and 20E). The disc 110 is then incubated at room temperature in a humidity chamber for 30 minutes. Unbound antibodies 188 are removed with a PBS wash, and the disc 110 is spin-dried to remove surface moisture (FIG. 20F). An adhesive member 118 is attached to the active layer 144 (FIG. 20G). A cap portion 116 (FIG. 2) may be formed from polycarbonate and is preferably coated with a reflective surface 146 on the bottom as best shown in FIG. 4. The cap portion 116 is integrally attached to adhesive member 118 thereby forming flow channels 130 within the disc (FIG. 20G). A blocking agent 204, such as StabilGuard®, is injected into each flow channel 130 to quickly and effectively block any remaining nonspecific binding sites of the active layer 144 (FIG. 20H). The disc 110 is incubated at room temperature in a humidity chamber for a predetermined time of preferably 30 minutes, and any remaining solution is completely removed via vacuum (FIG. 20I).

A second embodiment of the first implementation of the invention involves construction of a reflective disc 110 without the use of a cross-linking system to immobilize the capture agents within the flow channels 130 of the bio-disc 110. In this embodiment, a reference mark or calibration dot 202 is deposited over the first window 141 and non-biotinylated primary antibodies 188 (FIG. 18F) are deposited directly onto the active layer 144 (FIG. 20A) over each successive capture zone 140. The disc 110 is then incubated at room temperature in a humidity chamber for preferably 30 minutes (FIG. 20E). Unbound antibodies 188 are removed with a PBS wash, and the disc 110 is spin-dried to remove surface moisture (FIG. 20F). The adhesive member 118 is attached to the active layer 144. A cap portion 116 (FIG. 2) may be formed from polycarbonate and is preferably coated with a reflective surface 146 on the bottom as best shown in FIG. 4. The cap portion 116 is integrally attached to adhesive member 118 thereby forming flow channels 130 within the disc (FIG. 20G). A blocking agent 204, such as StabilGuard®, is injected into each flow channel 130 to quickly and effectively block any remaining nonspecific binding sites of the active layer 144 (FIG. 20H). The disc is incubated at room temperature in a humidity chamber for 30 minutes, and any remaining solution is completely removed via vacuum (FIG. 20I). FIG. 21 shows a cross-sectional view of the completed reflective bio-disc 110 without use of a cross-linking system.

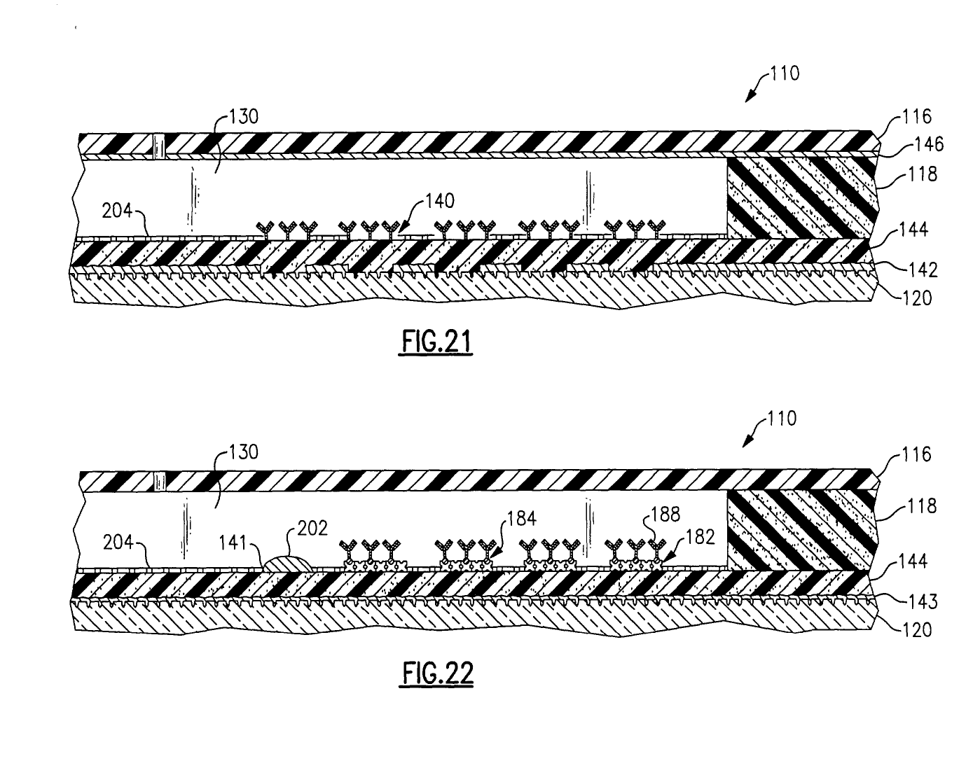

A third embodiment of the first implementation of the invention involves construction of a transmissive disc 110 utilizing a cross-linking system to immobilize the capture agents within the flow channels 130 of the bio-disc 110. In this embodiment, the substrate 120, semi-reflective layer 143 without viewing windows 200 (FIG. 20A), and active layer 144 are presented as shown in FIG. 5. Deposition of streptavidin 182, biotinylated primary antibodies 188, reference dot 202, and blocking agent 204 are as described above, and as shown in FIGS. 20B-20H. A respective adhesive member 118 is attached to the active layer 144. An optically transparent cap portion 116 (FIG. 5) may be formed from polycarbonate. The cap portion 116 is integrally attached to adhesive member 118 thereby forming flow channels 130 within the disc 110 (FIG. 20G). FIG. 22 shows a cross-sectional view of the completed transmissive bio-disc 110 utilizing a cross-linking system. It should be understood that a fourth embodiment of the first implementation may be constructed by those skilled in the art. The fourth embodiment involves construction of a transmissive disc 110 without the use of a cross-linking system to immobilize the capture agents within the flow channels of the bio-disc 110 (FIGS. 21 and 22).

Implementation II

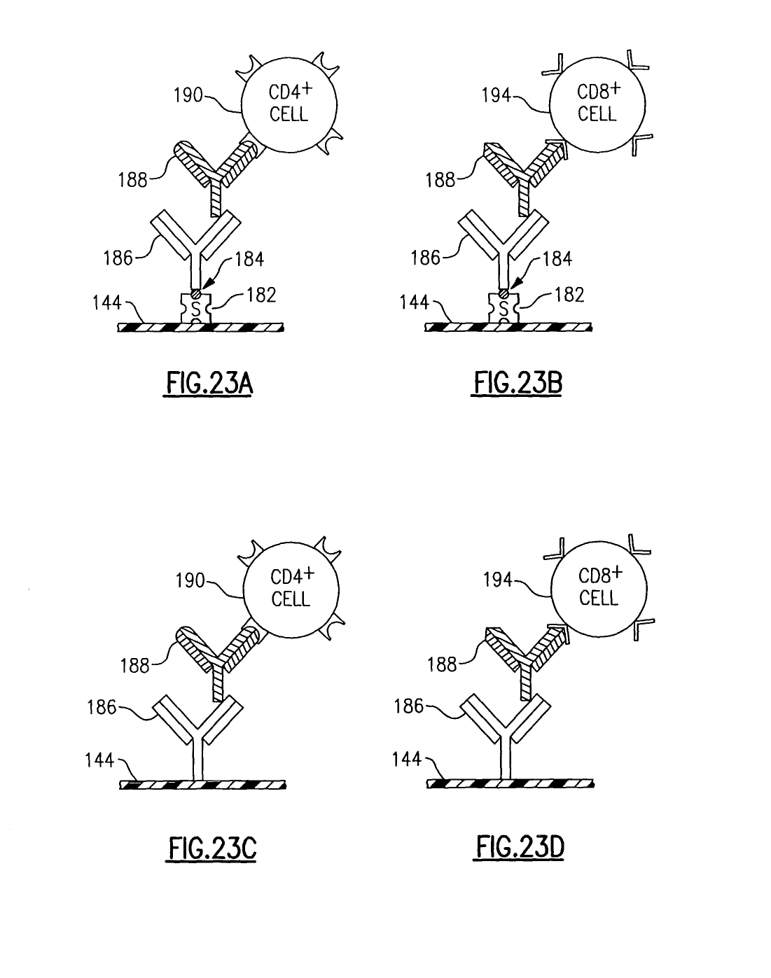

FIGS. 23A-23D are pictorial representations of analyte capture in a second implementation of the invention. FIGS. 23A and 23B show capture of CD4⁺ T-cells 190 and CD8⁺ T-cells 194 by primary antibodies 188 (FIG. 18F). The primary antibodies 188 are bound to biotinylated secondary antibodies 186 (FIG. 18E) immobilized on the active layer 144 of the bio-disc 110 (FIGS. 4 and 9) by the cross-linking agent streptavidin 182. FIGS. 23C and 23D show another embodiment of the same implementation of the invention without a cross-linking system. In this embodiment, the secondary antibodies 186 are immobilized directly on the active layer 144 of the bio-disc 110.

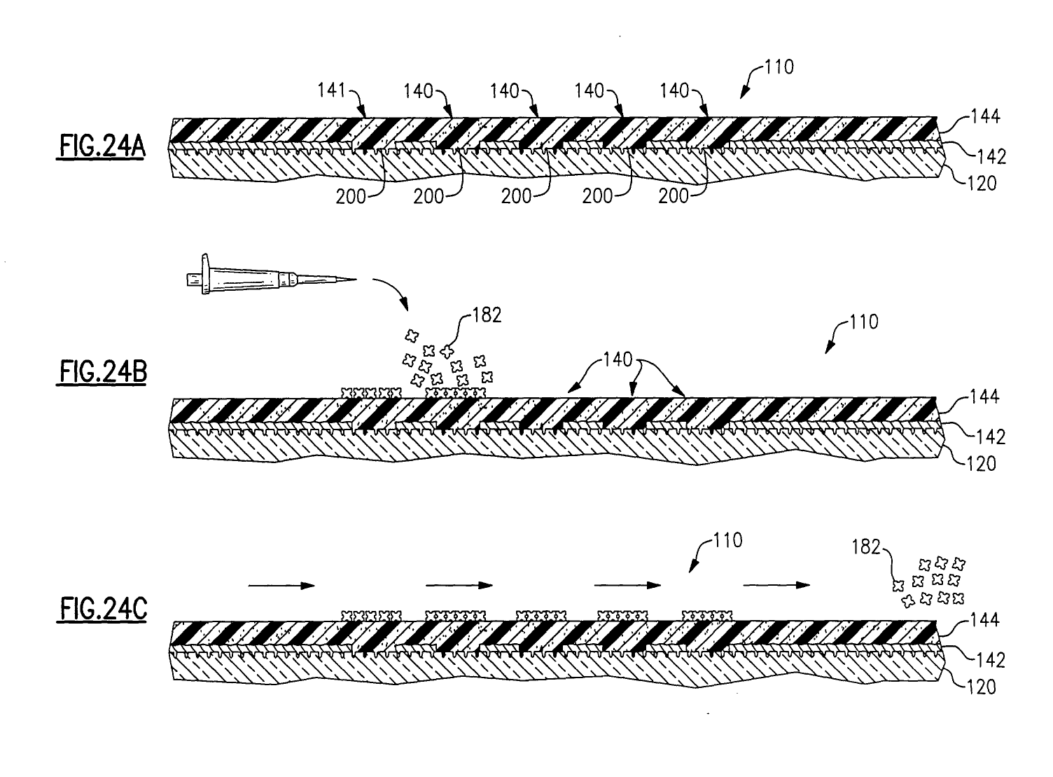

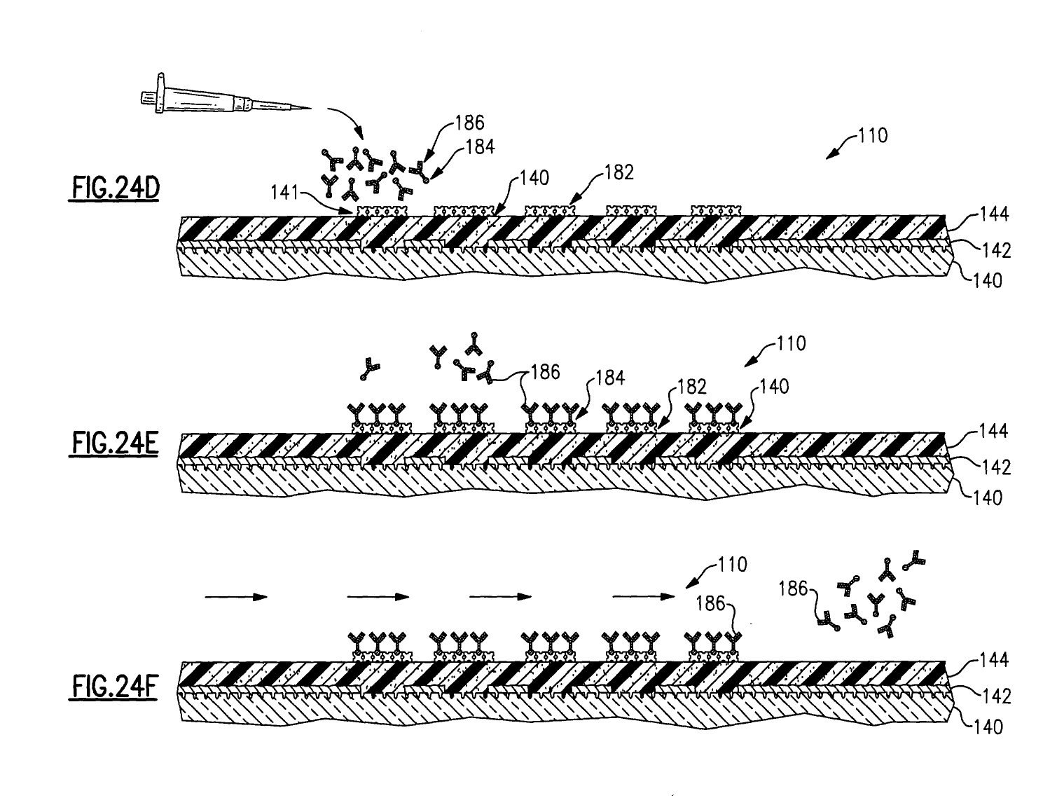

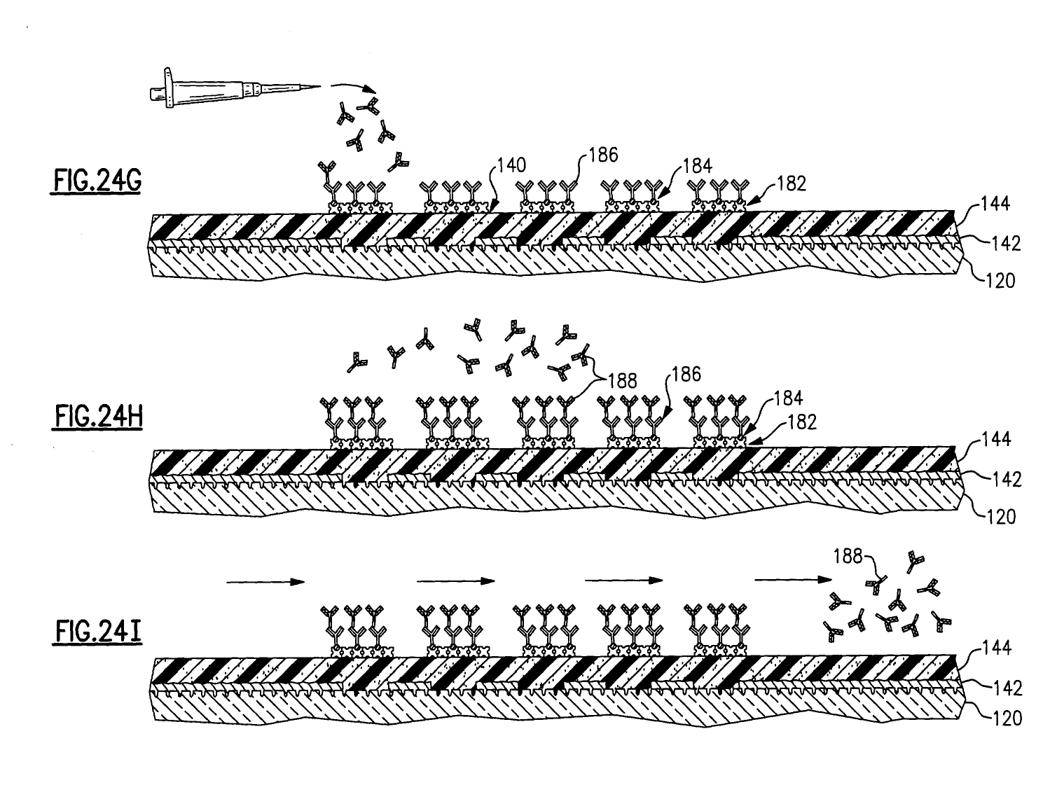

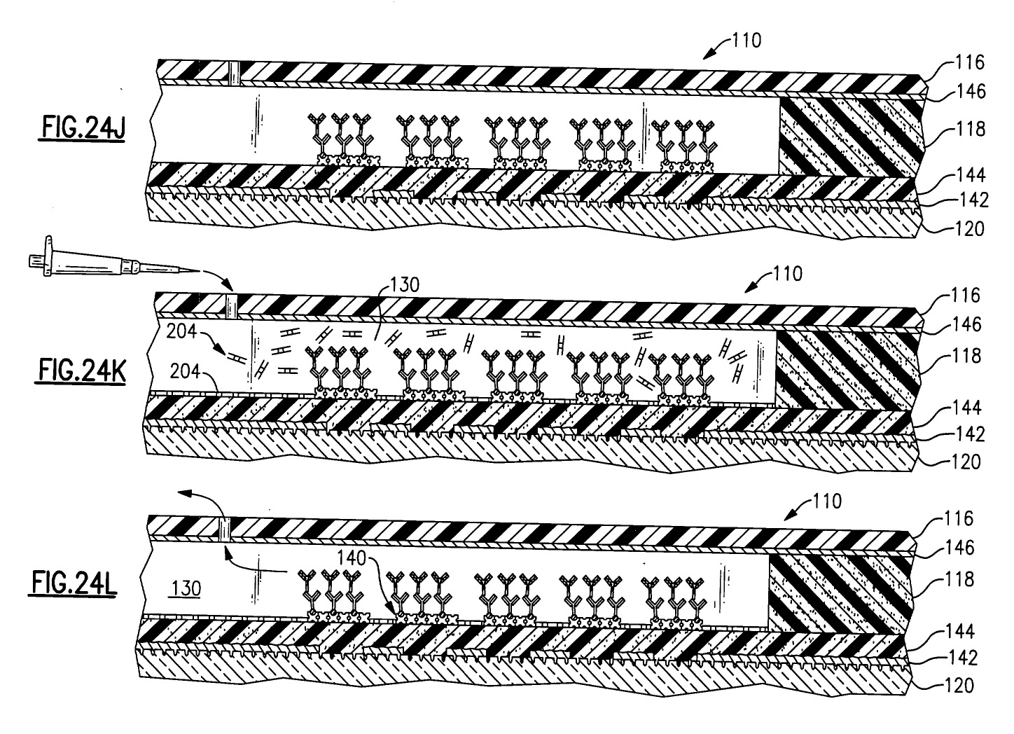

FIGS. 24A-24L are cross-sectional views showing construction of an embodiment of the second implementation of the invention. The first embodiment involves construction of a reflective disc utilizing a cross-linking system to immobilize the capture agents within the flow channels 130 of the bio-disc 110. Referring to FIG. 24A, a light-transparent substrate 120, a reflective layer 142, and an active layer 144 of an optical bio-disc 110 are shown. Portions of reflective layer 142 are removed (or openings were created when deposited) to produce viewing windows 200 through which light can be directed at the locations of capture zones 140 where the antibodies are to be affixed. FIG. 24A shows five such capture zones 140, the first thereof being designated as capture zone 141. The active layer 144 is preferably polystyrene, which is spin-coated over the reflective layer 142 to form a smooth surface with a thickness of about 40 to 300 microns. Streptavidin 182 is then deposited over each capture zone 140 and 141, and the disc 110 is incubated at room temperature in a humidity chamber for approximately 30 minutes (FIG. 24B). The disc 110 is washed to remove unbound streptavidin 182, and then spin-dried to completely remove moisture from the surface of the disc 110 (FIG. 24C). A reference mark or calibration dot 202 is deposited over the first capture zone 141 and biotinylated secondary antibodies 186 are deposited over each successive capture zone 140 (FIGS. 24D and 24E). The disc 110 is then incubated at room temperature in a humidity chamber for preferably 30 minutes (FIG. 24E). Unbound secondary antibodies 186 are removed with a PBS wash, and the disc 110 is spin-dried to remove surface moisture (FIG. 24F). Primary antibodies 188 (FIG. 18F) then are deposited over each capture zone 140 (FIG. 24G). The disc 110 is then incubated at room temperature in a humidity chamber for approximately 30 minutes (FIG. 24H). Unbound primary antibodies 188 are removed with a PBS wash, and the disc 110 is spin-dried to remove surface moisture (FIG. 24I). A respective adhesive member 118 is attached to the active layer 144. The cap portion 116 (FIG. 2) may be similarly formed from polycarbonate and is preferably coated with the reflective surface 146 on the bottom as best shown in FIG. 4. The cap portion 116 is integrally attached to adhesive member 118 thereby forming flow channels 130 within the disc (FIG. 24J). A blocking agent 204, such as StabilGuard®, is injected into each flow channel 130 to quickly and effectively block any remaining nonspecific binding sites of the active layer 144 (FIG. 24K). The disc 110 is incubated at room temperature in a humidity chamber for 30 minutes, and any remaining solution is completely removed via vacuum (FIG. 24L).

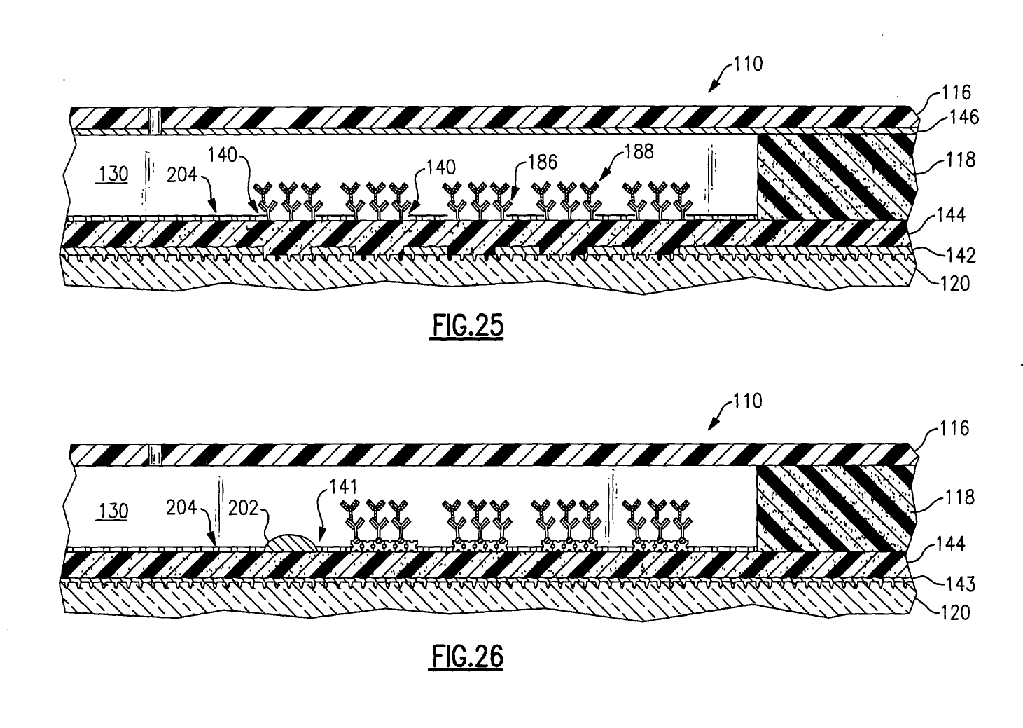

A second embodiment of the second implementation of the invention involves construction of a reflective disc 110 without the use of a cross-linking system to immobilize the capture agents within the flow channels 130 of the bio-disc 110. In this embodiment, a reference mark or calibration dot 202 is deposited over the first window 141 and non-biotinylated secondary antibodies 186 (FIG. 18D) are deposited directly onto the active layer 144 (FIG. 24A) over each successive capture zone 140. The disc 110 is then incubated at room temperature in a humidity chamber for 30 minutes (FIG. 24E). Unbound secondary antibodies 186 are removed with a PBS wash, and the disc 110 is spun-dried to remove surface moisture (FIG. 24F). Primary antibodies 188 (FIG. 18F) then are deposited over each capture zone 140 (FIG. 24G). The disc 110 is then incubated at room temperature in a humidity chamber for 30 minutes (FIG. 24H). Unbound primary antibodies 188 are removed with a PBS wash, and the disc 110 is spin-dried to remove surface moisture (FIG. 24I). An adhesive member 118 is attached to the active layer 144. As with the prior embodiments, the cap portion 116 of this embodiment may be formed from polycarbonate and is preferably coated with a reflective surface 146 on the bottom as best shown in FIG. 4. The cap portion 116 is integrally attached to adhesive member 118 thereby forming flow channels 130 within the disc (FIG. 24J). A blocking agent 204, such as StabilGuard®, is injected into each flow channel 130 to quickly and effectively block any remaining nonspecific binding sites of the active layer 144 (FIG. 24K). The disc is incubated at room temperature in a humidity chamber for preferably 30 minutes in this embodiment, and any remaining solution is completely removed via vacuum (FIG. 24L). FIG. 25 shows a cross-sectional view of the completed reflective bio-disc 110 without use of a cross-linking system.

A third embodiment of the second implementation of the invention involves construction of a transmissive disc 110 utilizing a cross-linking system to immobilize the capture agents within the flow channels 130 of the bio-disc 110. In this embodiment, the substrate 120, semi-reflective layer 143 without viewing windows 200 (FIG. 24A), and active layer 144 are presented as shown in FIG. 5. Deposition of streptavidin 182, biotinylated secondary antibodies 186, primary antibodies 188, reference dot 202, and blocking agent 204 are as described above, and as shown in FIGS. 24B-24K. The adhesive member 118 is attached to the active layer 144 in a similar manner. The optically transparent cap portion 116 (FIG. 5) may be formed from polycarbonate. The cap portion 116 is integrally attached to adhesive member 118 thereby forming flow channels 130 within the disc 110 (FIG. 24J). FIG. 26 shows a cross-sectional view of the completed transmissive bio-disc 110 utilizing a cross-linking system. It should be understood that a fourth embodiment of this second implementation may be constructed by those skilled in the art. The fourth embodiment involves construction of a transmissive disc 110 without the use of a cross-linking system to immobilize the capture agents within the flow channels of the bio-disc 110 (FIGS. 21 and 22).

Implementation III

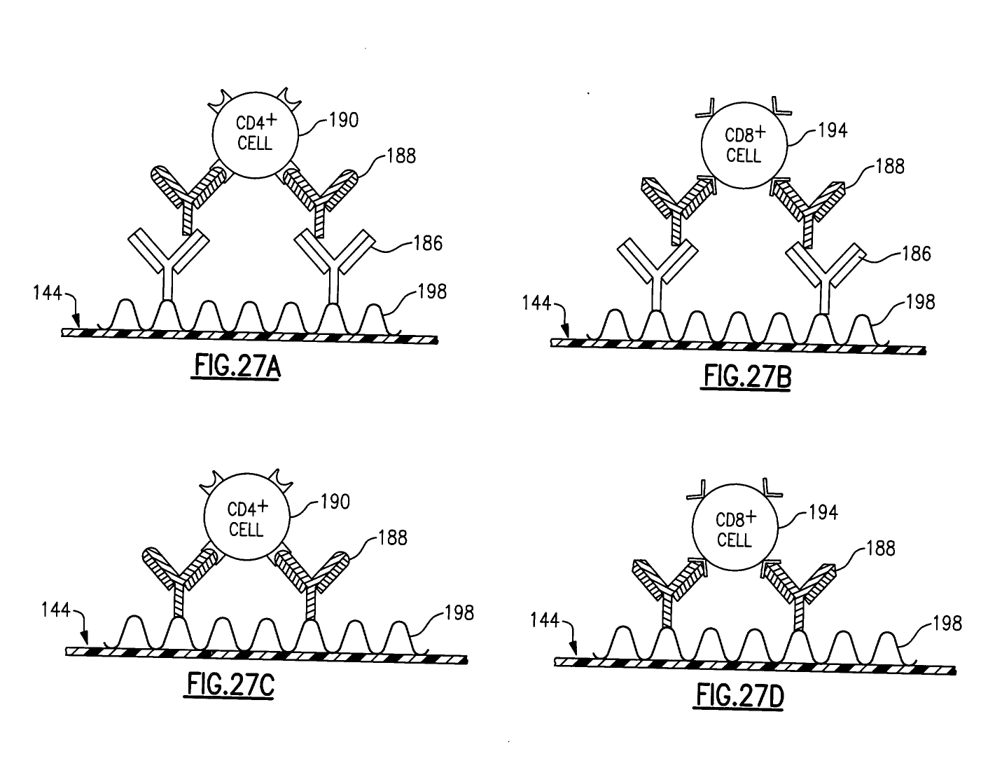

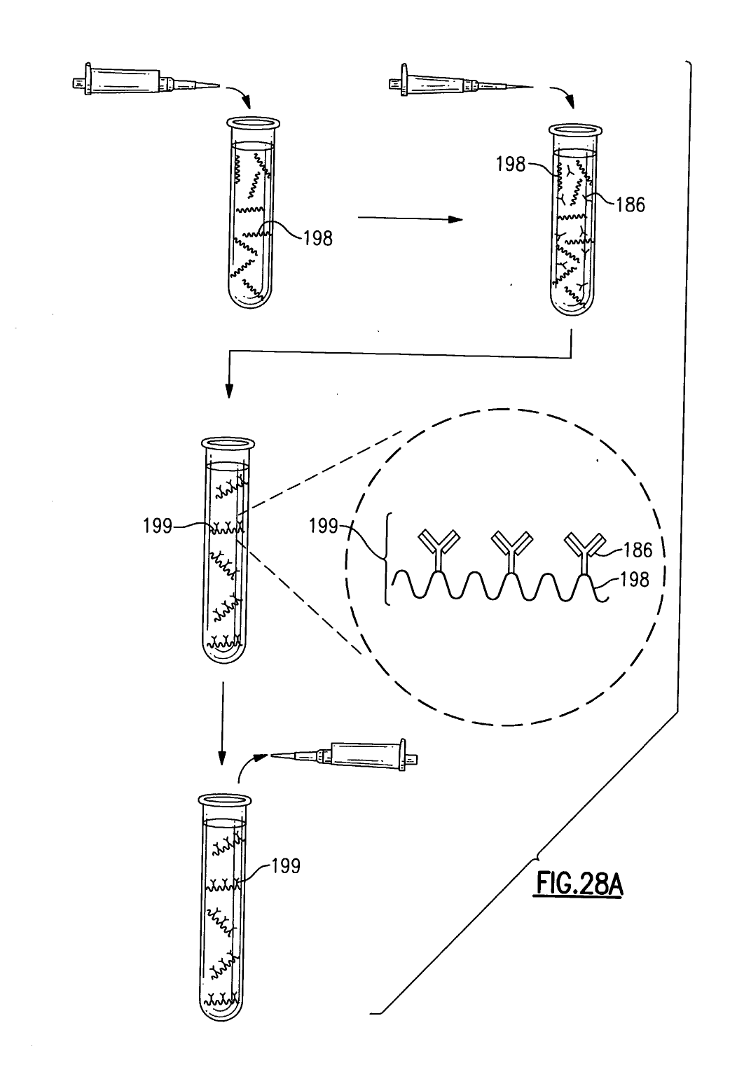

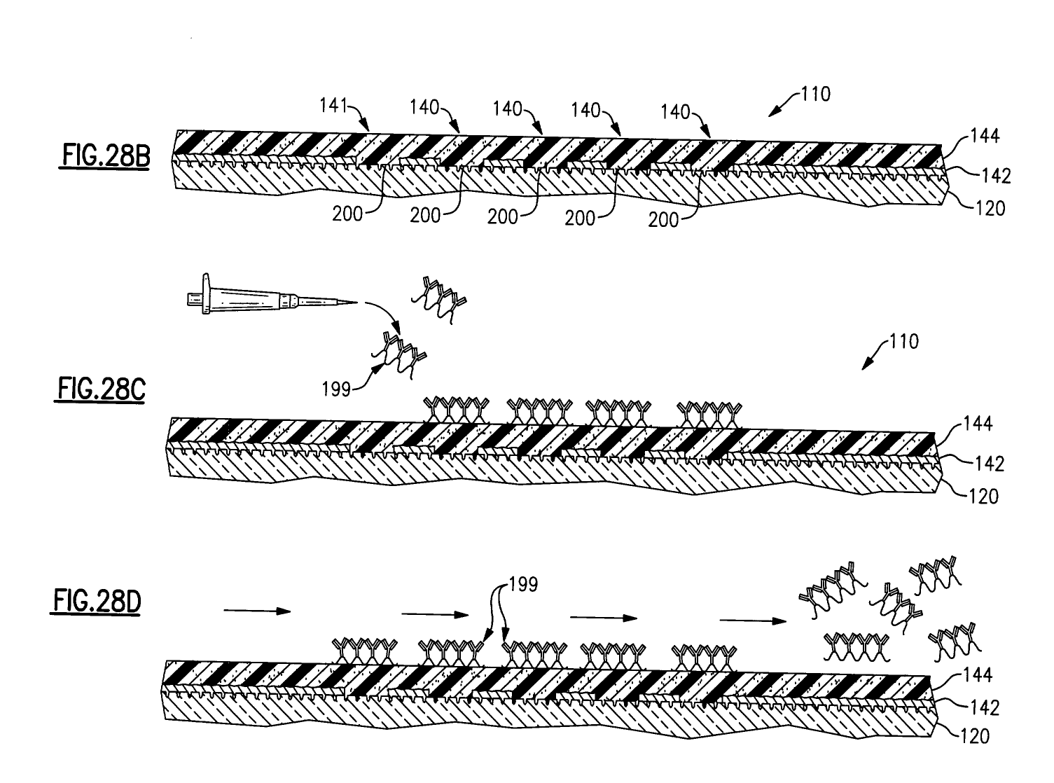

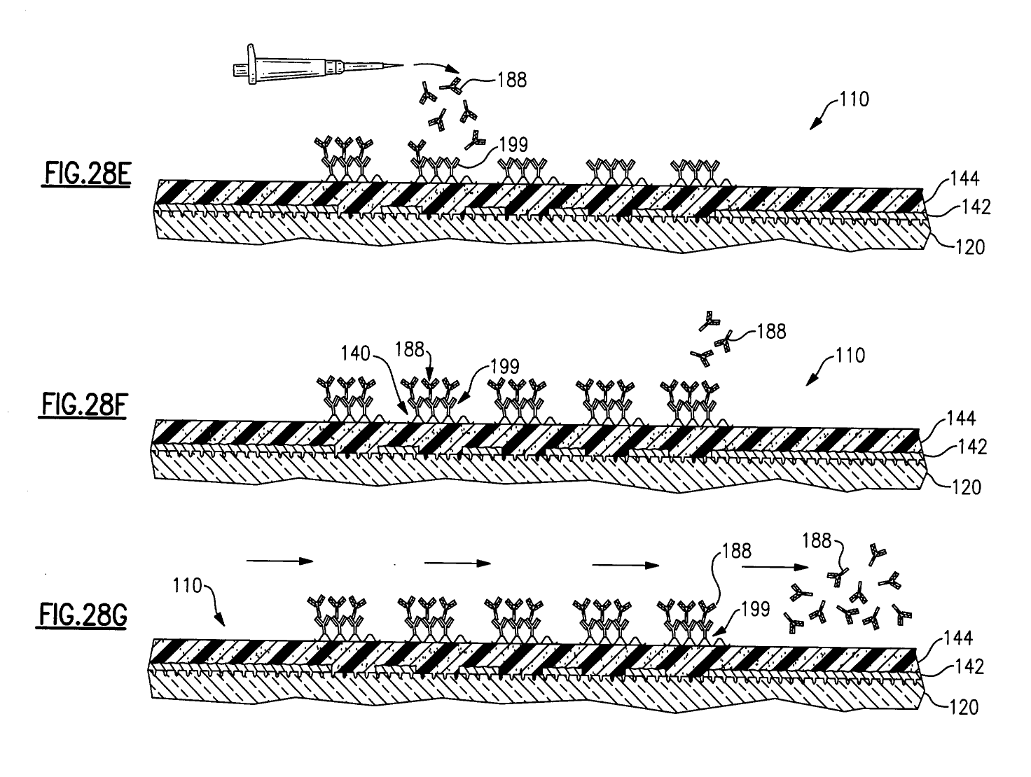

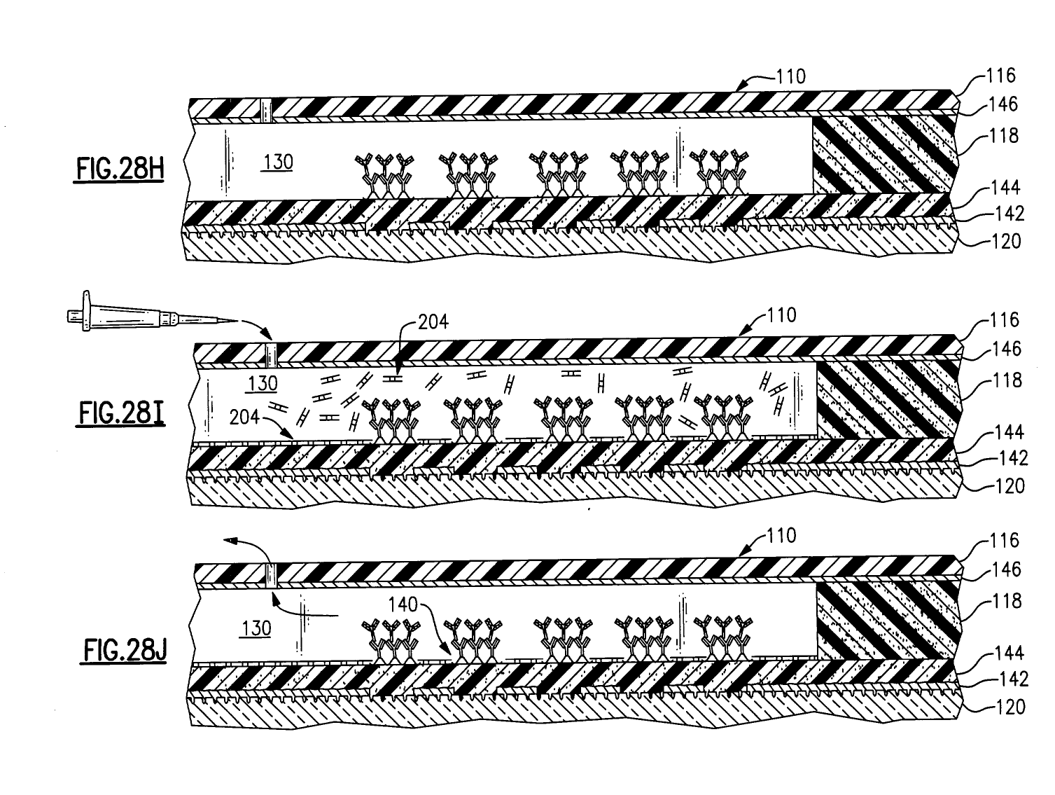

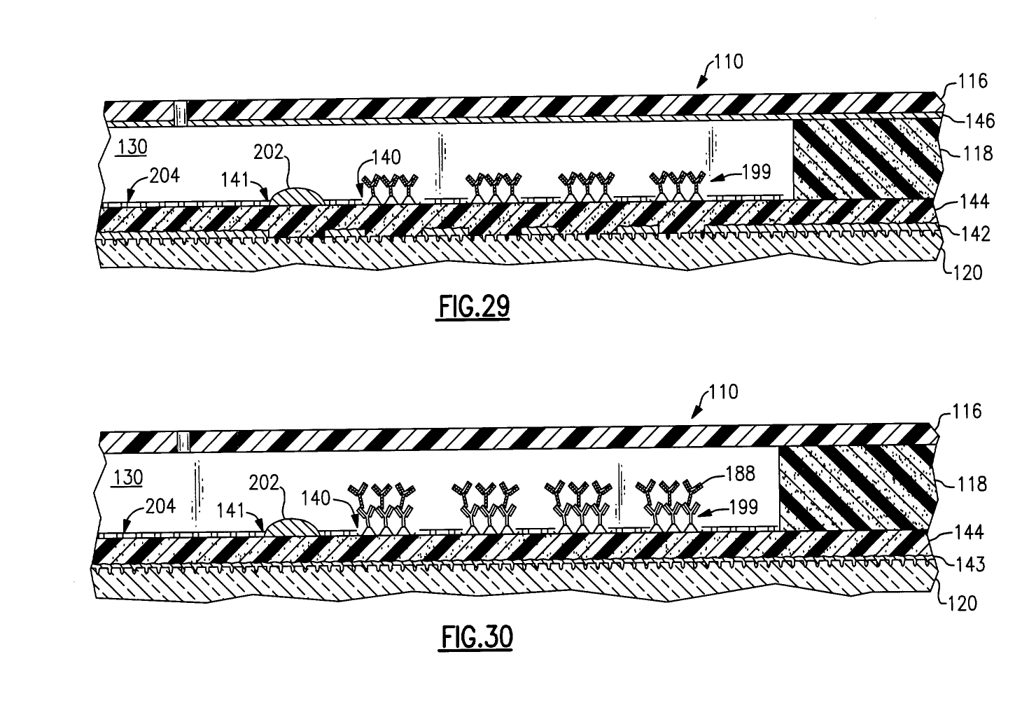

FIGS. 27A-27D are pictorial representations of analyte capture according to a third implementation of the present invention. FIGS. 27A and 27B show capture of CD4⁺ T-cells 190 and CD8⁺ T-cells 194 by primary antibodies 188 (FIG. 18F). The primary antibodies 188 are bound to secondary antibodies 186 (FIG. 18D), which are bound to a strand of DCHO 198 to form a DCHO-antibody complex 199 as shown below in FIG. 28A. The DCHO-antibody complex 199 may contain one or more antibodies cross-linked by the DCHO 198. The DCHO-antibody complex 199 is immobilized on the active layer 144 through binding of some of the secondary antibodies 186 to the active layer 144 of the bio-disc 110 (FIGS. 4 and 9). FIGS. 27C and 27D show another embodiment of the same implementation of the invention without the secondary antibodies 186. In this embodiment, the primary antibodies 188 are bound directly to the DCHO to form the DCHO-antibody complex 199, which is immobilized on the active layer 144 of the bio-disc 110.

FIG. 28A is a pictorial flow diagram showing preparation of antibody-DCHO complexes while FIGS. 28B-28J are multiple views showing construction of an embodiment of the third implementation of the invention. The first embodiment of this third implementation involves construction of a reflective disc utilizing the cross-linking agent DCHO to cross-link two or more capture agents. FIG. 28A shows preparation of the DCHO-antibody complex 199. Equal concentrations of DCHO 198 and secondary antibodies 186 are mixed and allowed to combine, thereby forming the DCHO-antibody (secondary) complex 199. Referring to FIG. 28B, a light-transparent substrate 120, a reflective layer 142, and an active layer 144 of an optical bio-disc 110 are shown. Portions of reflective layer 142 are removed (or opening created when deposited) to produce viewing windows 200 through which light can be directed at the locations of capture zones 140 where the antibodies are to be affixed. FIG. 28B shows five such capture zones 140, the first thereof indicated as capture zone 141. The active layer 144 is preferably polystyrene, which is spin-coated over the reflective layer 142 to form a smooth surface with a thickness of about 40 to 300 microns. DCHO-antibody complex (secondary) 199 is then deposited over each capture zone 140, and the disc 110 is incubated at room temperature in a humidity chamber for 30 minutes in this specific embodiment (FIG. 28C). The disc 110 is washed to remove unbound DCHO-antibody complex (secondary) 199, and then spin-dried to completely remove moisture from the surface of the disc 110 (FIG. 28D). A reference dot 202 is deposited over the first capture zone 141 and primary antibodies 188 are deposited over each successive capture zone 140 (FIG. 28E). The disc 110 is then incubated at room temperature in a humidity chamber for 30 minutes (FIG. 28F). Unbound primary antibodies 188 are removed with a PBS wash, and the disc 110 is spin-dried to remove surface moisture (FIG. 28G). The adhesive member or channel layer 118 is attached to the active layer 144. As with the above embodiments, the cap portion 116 generally shown in FIG. 2 may be formed from polycarbonate and is preferably coated with a reflective surface 146 on the bottom as best shown in FIG. 4. In the present embodiment, the cap portion 116 is integrally attached to adhesive member 118 thereby forming flow channels 130 within the disc as illustrated in FIG. 28H. A blocking agent 204, such as StabilGuard®, is injected into each flow channel 130 to quickly and effectively block any remaining nonspecific binding sites of the active layer 144. This step is shown in FIG. 28I. The disc 110 is incubated at room temperature in a humidity chamber for approximately 30 minutes in this embodiment, and any remaining solution is completely removed via vacuum as represented in FIG. 28J.

According to the manufacturing aspects of this invention, FIGS. 28A-28J also show a method of making an optical assay disc for performing a cluster designation count. This method of making an optical assay disc includes the steps of providing a cross-linker in a tube, adding a capture agent to the tube, allowing the cross-linker and the capture agent to combine (forming a complex), providing a substrate, coating the substrate with an active layer, depositing the complex onto the active layer, and attaching a cap portion to the active layer using an adhesive member. In this embodiment, the cross-linker is aldehyde-activated dextran. The capture agents are for binding with cell surface antigens. In an alternate embodiment, the capture agents are for binding with primary capture agents having a selective affinity for cell surface antigens. In a preferred embodiment, the cell surface antigens are independently selected from the CD family of antigens. In a more preferred embodiment, the cell surface antigens are independently selected from the group consisting of CD3, CD4, CD8, and CD45.

A second embodiment of the third implementation of the invention involves construction of a reflective disc 110 without the use of a secondary antibody to immobilize the primary capture agents within the flow channels 130 of the bio-disc 110. In this embodiment, equal concentrations of DCHO 198 and primary antibodies 188 are mixed and allowed to combine, thereby forming the DCHO-antibody (primary) complex 199 represented in FIG. 28A. A reference or calibration mark 202 is deposited over the first window 141 and the DCHO-antibody (primary) complex 199 are deposited onto the active layer 144 (FIG. 28C) over each successive capture zone 140. The disc 110 is then incubated at room temperature in a humidity chamber for preferably 30 minutes and unbound DCHO-antibody (primary) complex 199 is removed with a PBS wash. The disc 110 is spin-dried to remove surface moisture as illustrated in FIG. 28D. The adhesive member or channel layer 118 is similarly attached to the active layer 144. The cap portion 116 may be formed from polycarbonate and is preferably coated with a reflective surface 146 on the bottom as best shown in FIG. 4. The cap portion 116 is integrally attached to adhesive member 118 thereby forming flow channels 130 within the disc as shown in FIG. 28H. A blocking agent 204, such as StabilGuard®, is injected into each flow channel 130 to quickly and effectively block any remaining nonspecific binding sites of the active layer 144 (FIG. 28I). The disc is incubated at room temperature in a humidity chamber for approximately 30 minutes, and any remaining solution is completely removed via vacuum as illustrated in FIG. 28J. FIG. 29 shows a cross-sectional view of the completed reflective bio-disc 110 without use of secondary antibodies.

A third embodiment of the third implementation of the invention involves construction of a transmissive disc 110 utilizing the cross-linker DCHO to immobilize the capture agents within the flow channels 130 of the bio-disc 110. In this embodiment, the substrate 120, semi-reflective layer 143 without viewing windows 200 (FIG. 28B), and active layer 144 are presented as shown in FIG. 5. Deposition of DCHO-antibody (secondary) complex 199, primary antibodies 188, reference mark or dot 202, and blocking agent 204 are as described above, and as shown in FIGS. 28C-28I. The adhesive member or channel layer 118 is attached to the active layer 144. An optically transparent cap portion 116 (FIG. 5) may be formed from polycarbonate. The cap portion 116 is integrally attached to adhesive member 118 thereby forming flow channels 130 within the disc 110. FIG. 30 shows a cross-sectional view of the completed transmissive bio-disc 110 utilizing a DCHO-antibody (secondary) complex 199 and primary antibodies 188. It should be understood that a fourth embodiment of the third implementation may be constructed by those skilled in the art. The fourth embodiment involves construction of a transmissive disc 110 without the use secondary antibodies to immobilize the primary capture agents within the flow channels of the bio-disc 110 (FIGS. 29 and 30). Other embodiments may involve use of streptavidin 182 (FIG. 18A) and biotin 184 (FIG. 18B) to immobilize primary or secondary antibodies to the active layer 144 of the bio-disc 110. Immobilized antibodies (both primary and secondary) can thereafter be complexed with DCHO 198 for increased concentrations of capture agents within the flow channels 130 of the bio-disc 110.

Implementation IV

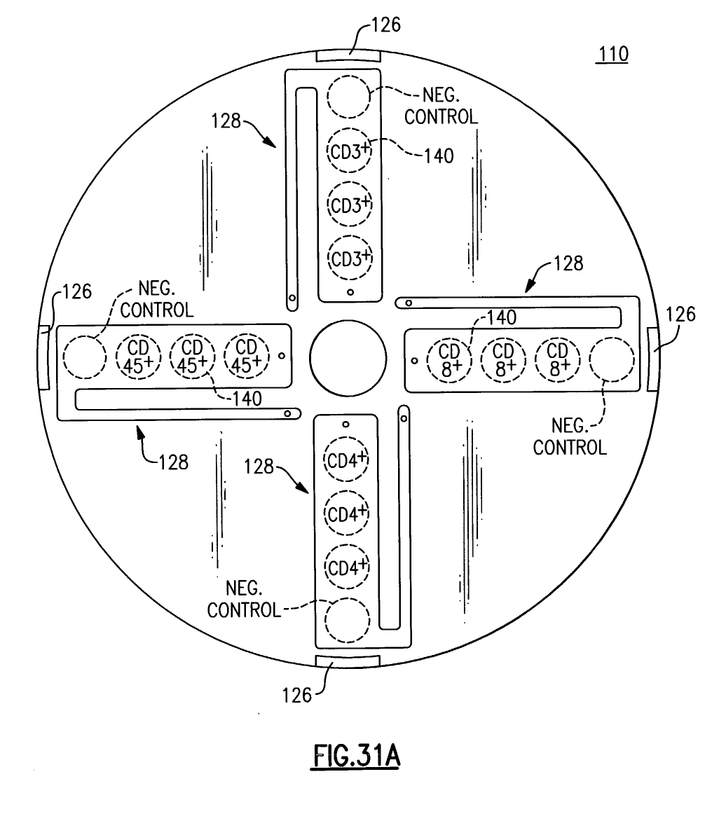

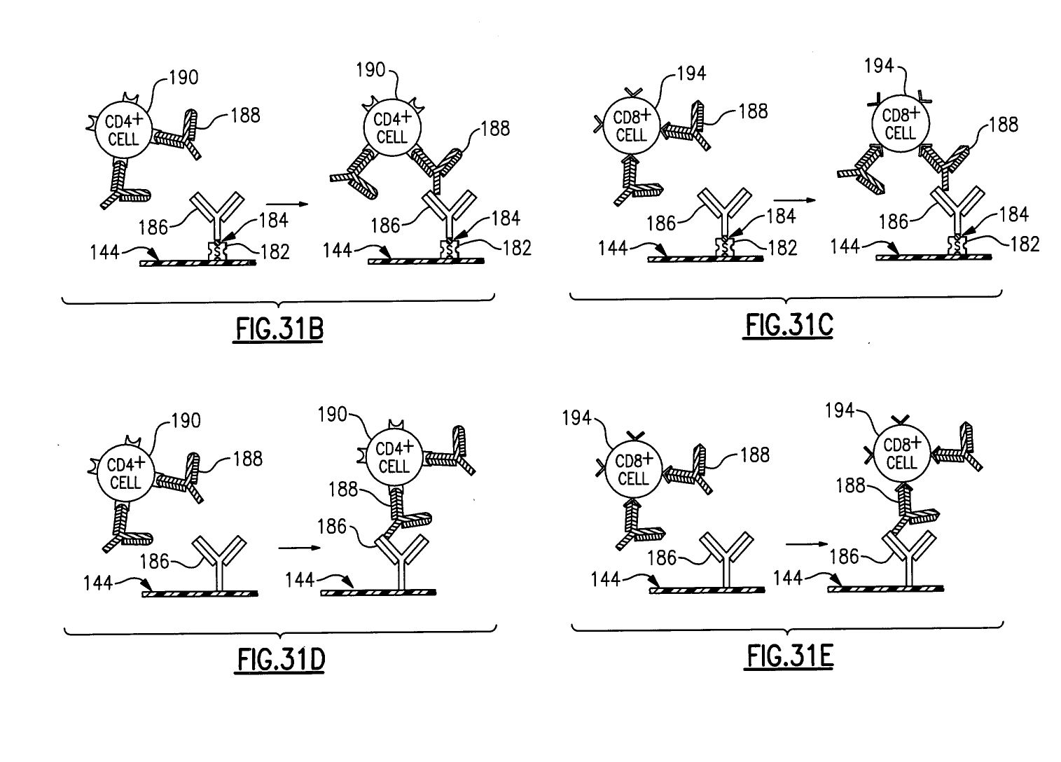

FIG. 31A is a top plan view of an optical bio-disc 110 showing four fluidic circuits 128 each having several capture zones 140 for a specific cell surface marker and a negative control zone. As shown, each fluidic circuit is dedicated to having capture zones 140 specifically directed to CD3, CD4, CD8, and CD45. Any other desired pattern or combination of cell surface antigens such as, for example, other CD markers or cell surface markers may be employed. In this particular implementation, for each individual fluidic circuit it is necessary to have only a single common capture agent in each of the capture zones 140. The disc shown in FIG. 31A is specifically suited for use with the cell capture chemistries and methods described below in FIGS. 31B-31E, 44A-44D, 45, and 46. The bio-disc 110 represented in FIG. 31A may be either a reflective or transmissive disc.

Turning now to FIGS. 31B-31E, there is shown pictorial representations of analyte capture in a fourth implementation of the present invention. FIGS. 31B and 31C show capture by secondary antibodies 186 of CD4⁺ cells 190 and CD8⁺ cells 194 that were previously combined with primary antibodies 188 (FIG. 18F) to form primary antibody-cell complexes. The primary antibodies 188 are bound to biotinylated secondary antibodies 186 (FIG. 18E) immobilized on the active layer 144 of the bio-disc 110 (FIGS. 4 and 9) by the cross-linking agent streptavidin 182. FIGS. 31D and 31E show another embodiment of the same implementation of the invention without a cross-linking system. In this embodiment, the secondary antibodies 186 are immobilized directly on the active layer 144 of the bio-disc 110.

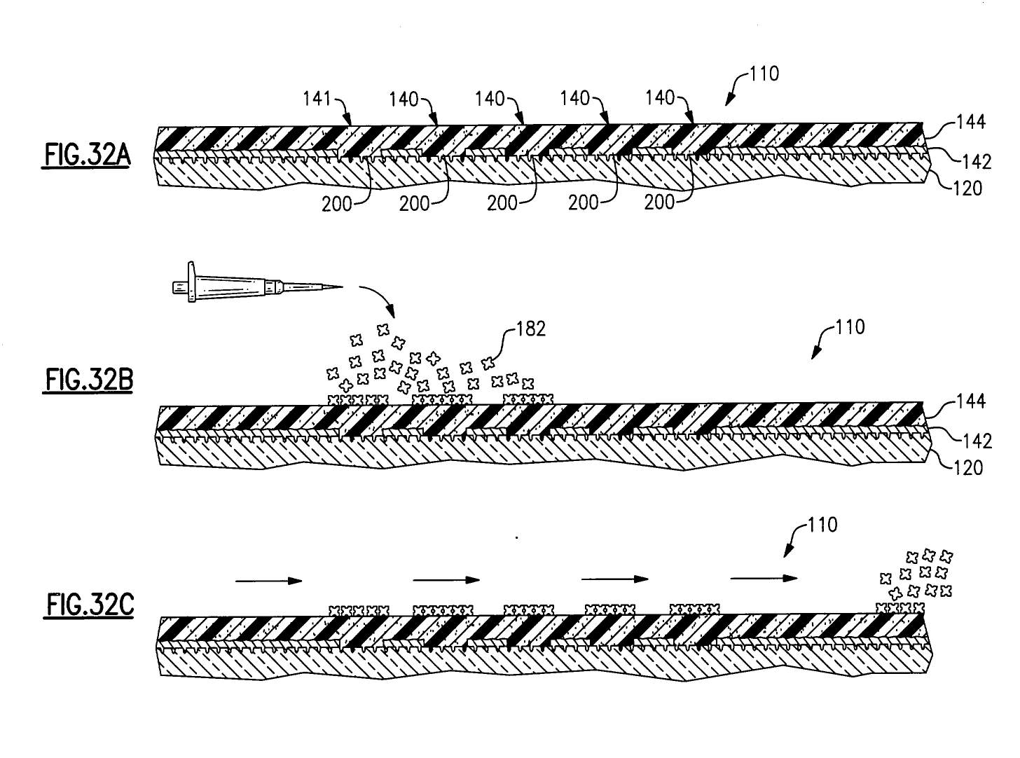

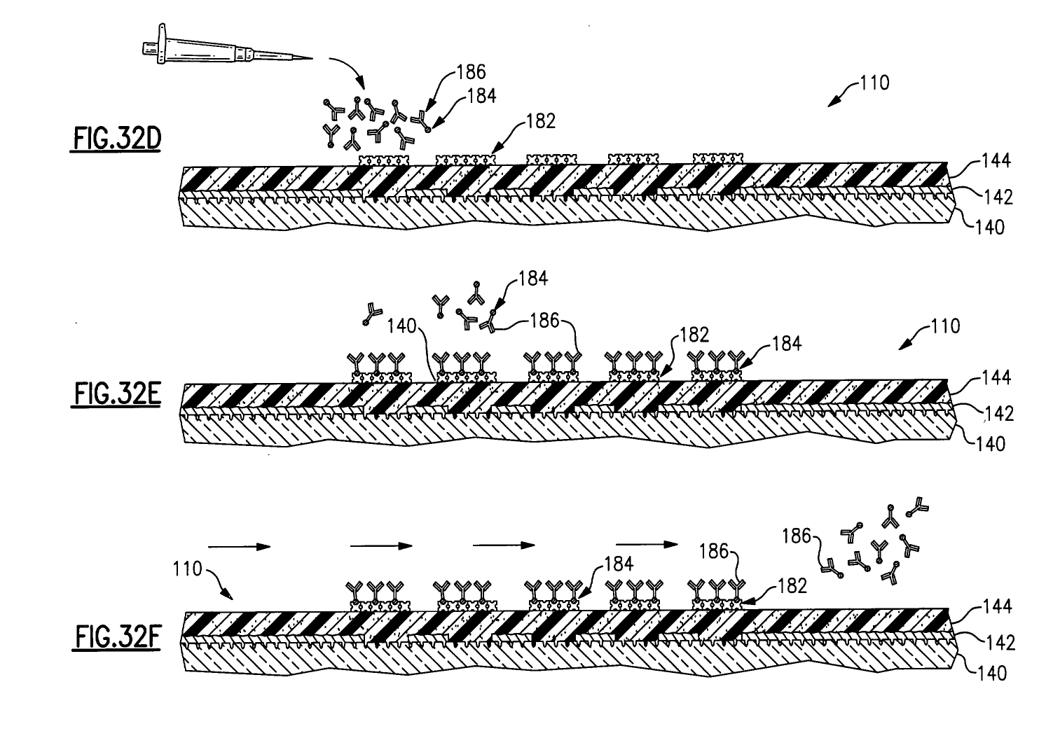

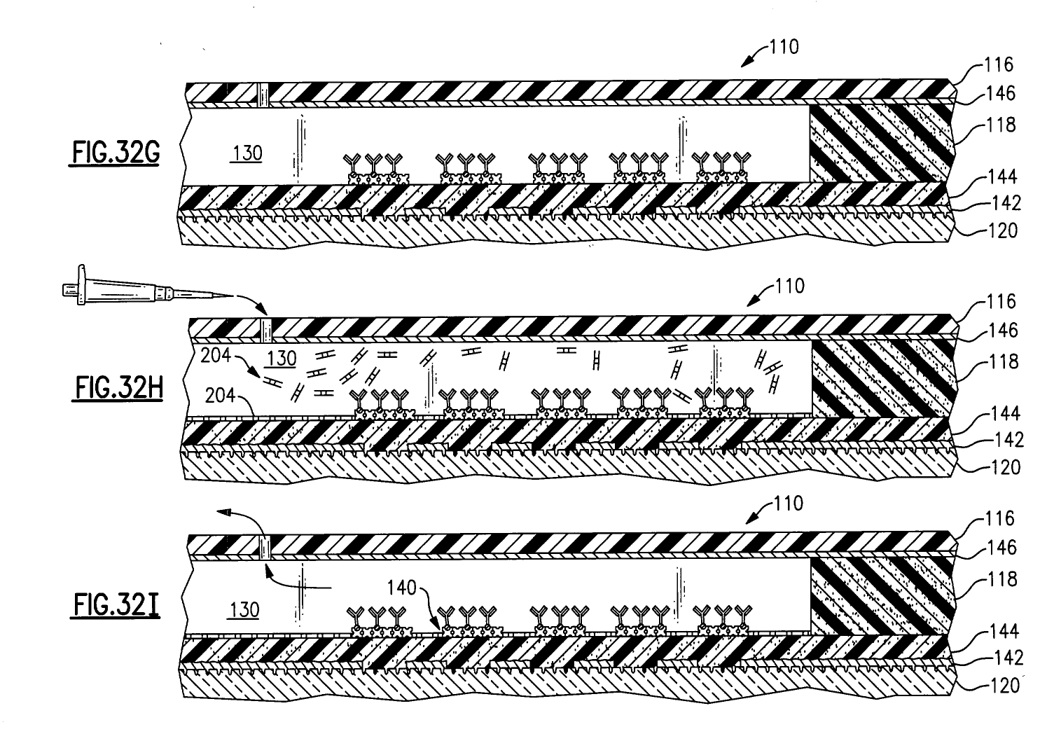

FIGS. 32A-32I are cross-sectional views showing construction of an embodiment of the fourth implementation of the invention. The first embodiment involves construction of a reflective disc utilizing a cross-linking system to immobilize the capture agents within the flow channels of the bio-disc. Referring to FIG. 32A, a light-transparent substrate 120, a reflective layer 142, and an active layer 144 of an optical bio-disc 110 are shown. Portions of reflective layer 142 are removed (or openings were created when deposited) to produce viewing windows 200 through which light can be directed at the locations of capture zones 140 where the antibodies are to be affixed. FIG. 32A shows five such capture zones 140, the first thereof designated as capture zone 141. The active layer 144 is preferably polystyrene, which is spin-coated over the reflective layer 142 to form a smooth surface with a thickness of about 40 to 300 microns. Streptavidin 182 is then deposited over each capture zone 140 and 141, and the disc 110 is incubated at room temperature in a humidity chamber for approximately 30 minutes as represented in FIG. 32B. The disc 110 is washed to remove unbound streptavidin 182, and then spin-dried to completely remove moisture from the surface of the disc 110 as illustrated in FIG. 32C. A reference mark or calibration dot 202 is deposited over the first capture zone 141 and biotinylated secondary antibodies 186 are deposited over each successive capture zone 140 as shown in FIGS. 32D and 32E. The disc 110 is then incubated at room temperature in a humidity chamber for approximately 30 minutes in this embodiment. This step is illustrated in FIG. 32E. Unbound secondary antibodies 186 are removed with a PBS wash, and the disc 110 is spin-dried to remove surface moisture. This step of the present method is represented in FIG. 32F. An adhesive member or channel layer 118 is similarly applied to the active layer 144. A respective cap portion 116 is preferably formed from polycarbonate and coated with a reflective surface 146 on the bottom as best shown in FIG. 4. The cap portion 116 is integrally attached to adhesive member 118 thereby forming flow channels 130 within the disc as shown in FIG. 32G. A blocking agent 204, such as StabilGuard®, is injected into each flow channel 130 to quickly and effectively block any remaining nonspecific binding sites of the active layer 144. This step is shown in FIG. 32H. The disc 110 is incubated at room temperature in a humidity chamber for preferably 30 minutes, and any remaining solution is completely removed via vacuum as represented in FIG. 32I.

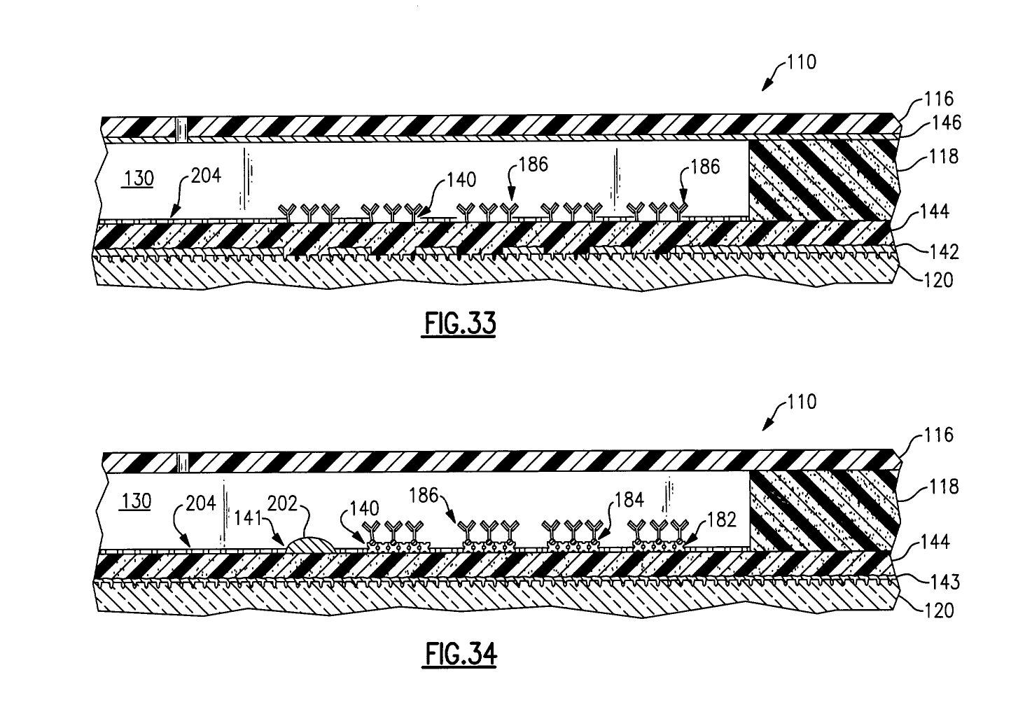

A second embodiment of the fourth implementation of the invention involves construction of a reflective disc 110 without the use of a cross-linking system to immobilize the capture agents within the flow channels 130 of the bio-disc 110. In this embodiment, a reference mark or calibration dot 202 is deposited over the first window 141 and non-biotinylated secondary antibodies 186, shown generally in FIG. 18D, are deposited directly onto the active layer 144 over each successive capture zone 140. The disc 110 is then incubated at room temperature in a humidity chamber for 30 minutes. Unbound secondary antibodies 186 are removed with a PBS wash, and the disc 110 is spin-dried to remove surface moisture. An adhesive member 118 is attached to the active layer 144. A cap portion 116 may be formed from polycarbonate and is preferably coated with a reflective surface 146 on the bottom as best shown in FIG. 4. The cap portion 116 is integrally attached to adhesive member 118 thereby forming flow channels 130 within the disc. A blocking agent 204, such as StabilGuard®, is injected into each flow channel 130 to quickly and effectively block any remaining nonspecific binding sites of the active layer 144. The disc is incubated at room temperature in a humidity chamber for approximately 30 minutes, and any remaining solution is completely removed via vacuum. FIG. 33 shows a cross-sectional view of the completed reflective bio-disc 110 without use of a cross-linking system.

A third embodiment of the fourth implementation of the invention involves construction of a transmissive disc 110 utilizing a cross-linking system to immobilize the capture agents within the flow channels 130 of the bio-disc 110. In this embodiment, the substrate 120, semi-reflective layer 143 without viewing windows 200 (FIG. 32A), and active layer 144 are presented as shown in FIG. 5. Deposition of streptavidin 182, biotinylated primary antibodies 188, reference mark 202, and blocking agent 204 are as described above, and as shown in FIGS. 32B-32H. An adhesive member 118 is attached to the active layer 144. An optically transparent cap portion 116 (FIG. 5) may be formed from polycarbonate. The cap portion 116 is integrally attached to adhesive member 118 thereby forming flow channels 130 within the disc 110 (FIG. 32G). FIG. 34 shows a cross-sectional view of the completed transmissive bio-disc 110 utilizing a cross-linking system. It should be understood that a fourth embodiment of the fourth implementation may be constructed by those skilled in the art. The fourth embodiment involves construction of a transmissive disc 110 without the use of a cross-linking system to immobilize the secondary capture agents within the flow channels of the bio-disc 110 (FIGS. 33 and 34).

Cluster Designation Count—Implementation I

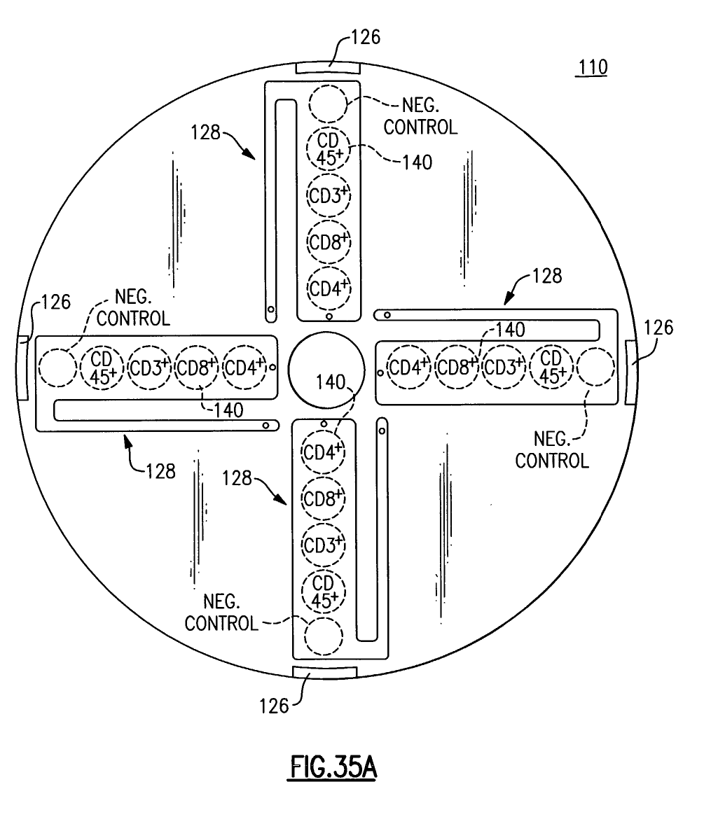

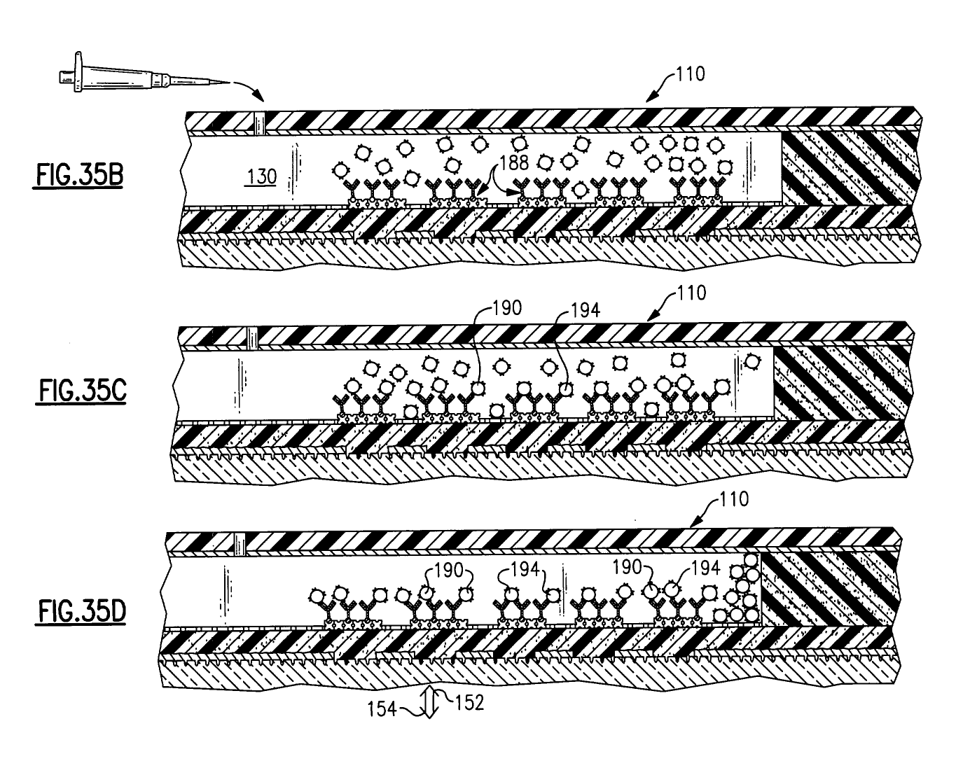

FIG. 35A is a top plan view of an optical bio-disc 110 showing four fluidic circuits 128 each having several capture zones 140 for four different specific cell surface markers and a negative control zone. As shown, each fluidic circuit is dedicated to having four capture zones 140 each one specifically directed to CD4, CD8, CD3, and CD45. Any other desired pattern or combination of cell surface antigens such as, for example, other CD markers or cell surface markers may be employed. In this particular implementation, there is no restriction that for each individual fluidic circuit it is necessary to have only a single common capture agent in each of the capture zones 140. On the contrary, in this implementation, it is desirable to have capture zones with different capture agents. These capture zones may be arranged, for example, in an array format having at least a 2-by-2 matrix. The disc shown in FIG. 35A is specifically suited for use with the cell capture chemistries and methods described below in FIGS. 35B-35D, 36, 37, 38A-38C, 39, 40, 41A-41C, 42, and 43. The bio-disc 110 represented in FIG. 35A may be either a reflective or transmissive disc.

Referring next to FIGS. 35B-35D, analysis of a purified and washed MNC sample (FIG. 17A) using a bio-disc of the first implementation of the invention is shown. The flow channels 130 of a bio-disc 110 are flooded with the MNC sample as represented in FIG. 35B. Capillary action, pressure applied with an external applicator, and/or centrifugal force (i.e., the force on a body in curvilinear motion directed away from the center or curvature or axis of rotation) act upon the test sample to achieve contact with the primary antibodies 188. As shown in FIG. 35C, the primary antibodies come into contact with and then capture any CD4⁺ cells 190 and CD8⁺ cells 194 present within the test sample. The optical drive motor 162 (FIG. 10) spins the disc, which clears the capture zones 140 (FIG. 20I) of unbound T-cells. The incident beam 152 of an optical disc drive 112 (FIG. 1) interacts with the captured CD4⁺ cells 190 and CD8⁺ cells 194, FIG. 35D, and the return beam 154 is reflected to the detector 157 (FIG. 10) for processing and analysis.

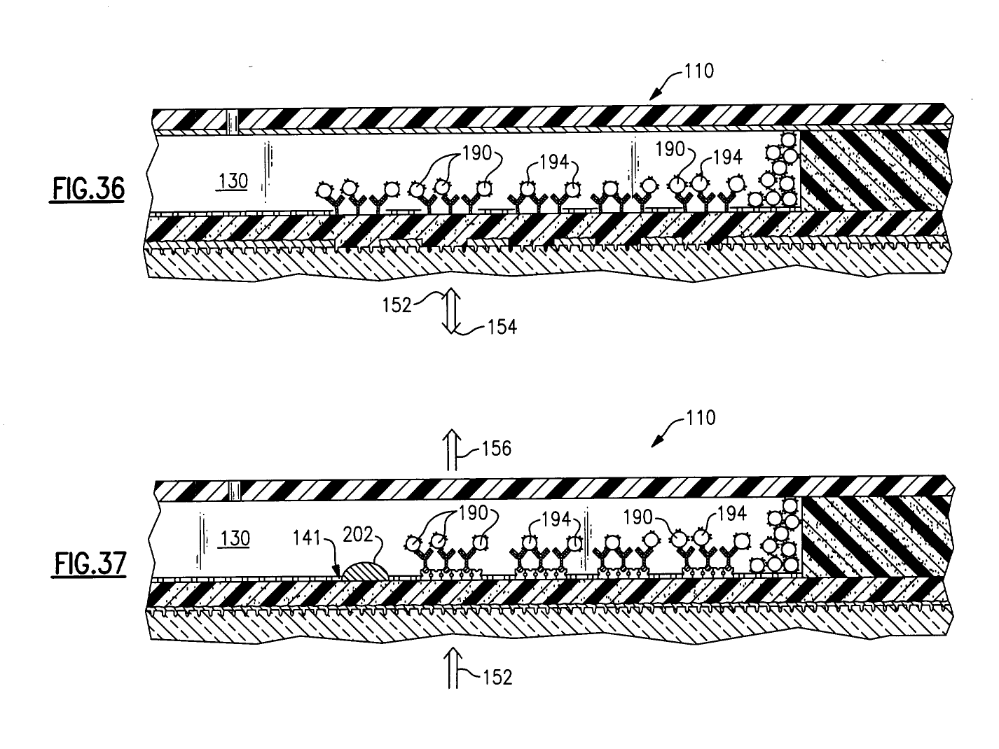

FIG. 36 shows analysis of the same test sample from FIGS. 35B-35D, using the second embodiment of the first implementation of the invention. The flow channels 130 of a bio-disc 110 are flooded with the MNC sample (FIG. 35B). Capillary action, pressure applied with an external applicator, and/or centrifugal force (i.e., the force on a body in curvilinear motion directed away from the center or curvature or axis of rotation) act upon the test sample to achieve contact with the primary antibodies 188. The primary antibodies come into contact with, and capture any CD4⁺ T-cells 190 and CD8⁺ T-cells 194 present within the test sample (FIG. 35C). The optical drive motor 162 (FIG. 10) spins the disc, which clears the capture zones 140 (FIG. 20I) of unbound T-cells. The incident beam 152 of an optical disc drive 112 (FIG. 1) interacts with the captured CD4⁺ T-cells 190 and CD8⁺ T-cells 194 (FIG. 35D) and the return beam 154 is reflected to the detector 157 (FIG. 10) for processing and analysis.

FIG. 37 shows analysis of the same test sample from FIGS. 35B-35D, using the third embodiment of the first implementation of the invention. The flow channels 130 of a bio-disc 110 are flooded with the MNC sample (FIG. 35B). Capillary action, pressure applied with an external applicator, and/or centrifugal force (i.e., the force on a body in curvilinear motion directed away from the center or curvature or axis of rotation) act upon the test sample to achieve contact with the primary antibodies 188. The primary antibodies come into contact with and then capture any CD4⁺ T-cells 190 and CD8⁺ T-cells 194 present within the test sample (FIG. 35C). The optical drive motor 162 (FIG. 10) spins the disc, which clears the capture zones 140 (FIG. 20I) of unbound T-cells. The incident beam 152 of an optical disc drive 112 (FIG. 1) interacts with the captured CD4⁺ T-cells 190 and CD8⁺ T-cells 194 (FIG. 35D) and the transmitted beam 156is passed to the detector 157 (FIG. 10) for processing and analysis.

Cluster Designation Count—Implementation II

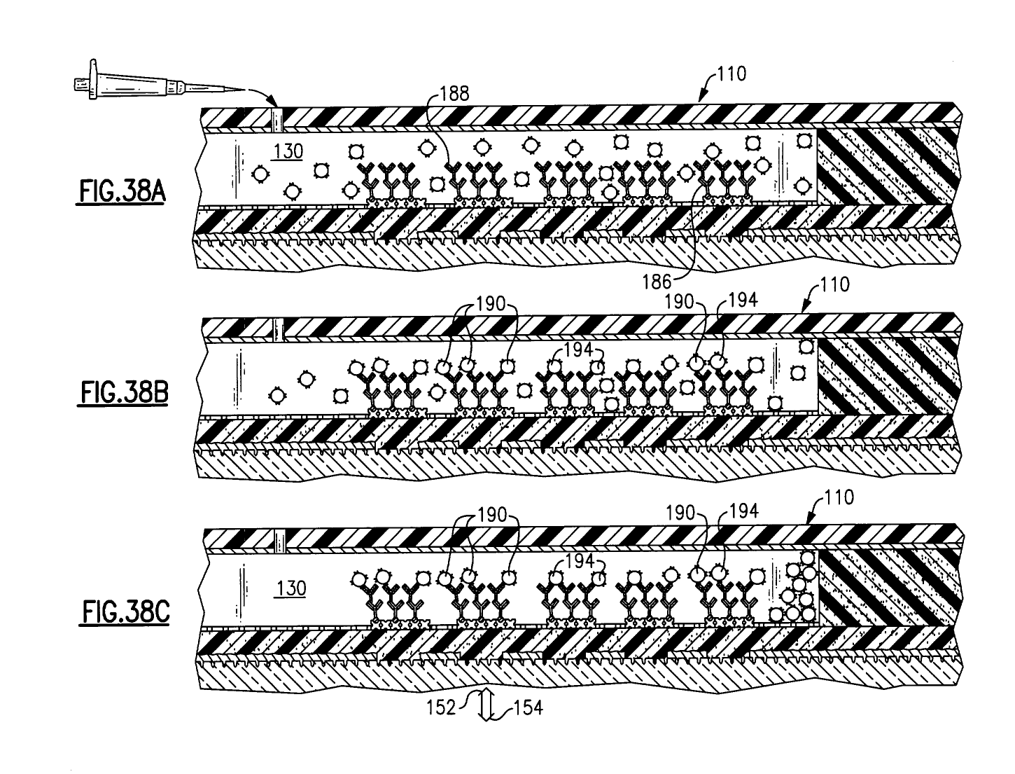

With reference now to FIGS. 38A-38C, analysis of a purified and washed MNC sample (FIG. 17A) using a bio-disc of the second implementation of the invention is shown. The flow channels 130 of a bio-disc 110 are flooded with the MNC sample as shown in FIG. 38A. Capillary action, pressure applied with an external applicator, and/or centrifugal force (i.e., the force on a body in curvilinear motion directed away from the center or curvature or axis of rotation) act upon the test sample to achieve contact with the primary antibodies 188. The primary antibodies come into contact with, and capture any CD4⁺ T-cells 190 and CD8⁺ T-cells 194 present within the test sample. This step is illustrated in FIG. 38B. The optical drive motor 162 (FIG. 10) spins the disc, which clears the capture zones 140 (FIG. 24L) of unbound T-cells. The incident beam 152 of an optical disc drive 112 (FIG. 1) interacts with the captured CD4⁺ T-cells 190 and CD8⁺ T-cells 194, FIG. 38C, and the return beam 154 is reflected to the detector 157 (FIG. 10) for processing and analysis.

FIG. 39 shows analysis of the same test sample from FIGS. 38A-38C, using the second embodiment of the second implementation of the invention. The flow channels 130 of a bio-disc 110 are flooded with the MNC sample (FIG. 38A). Capillary action, pressure applied with an external applicator, and/or centrifugal force (i.e., the force on a body in curvilinear motion directed away from the center or curvature or axis of rotation) act upon the test sample to achieve contact with the primary antibodies 188. The primary antibodies come into contact with, and capture any CD4⁺ T-cells 190 and CD8⁺ T-cells 194 present within the test sample (FIG. 38B). The optical drive motor 162 (FIG. 10) spins the disc, which clears the capture zones 140 (FIG. 24L) of unbound T-cells. The incident beam 152 of an optical disc drive 112 (FIG. 1) interacts with the captured CD4⁺ T-cells 190 and CD8⁺ T-cells 194 and (FIG. 38C) the return beam 154 is reflected to the detector 157 (FIG. 10) for processing and analysis.

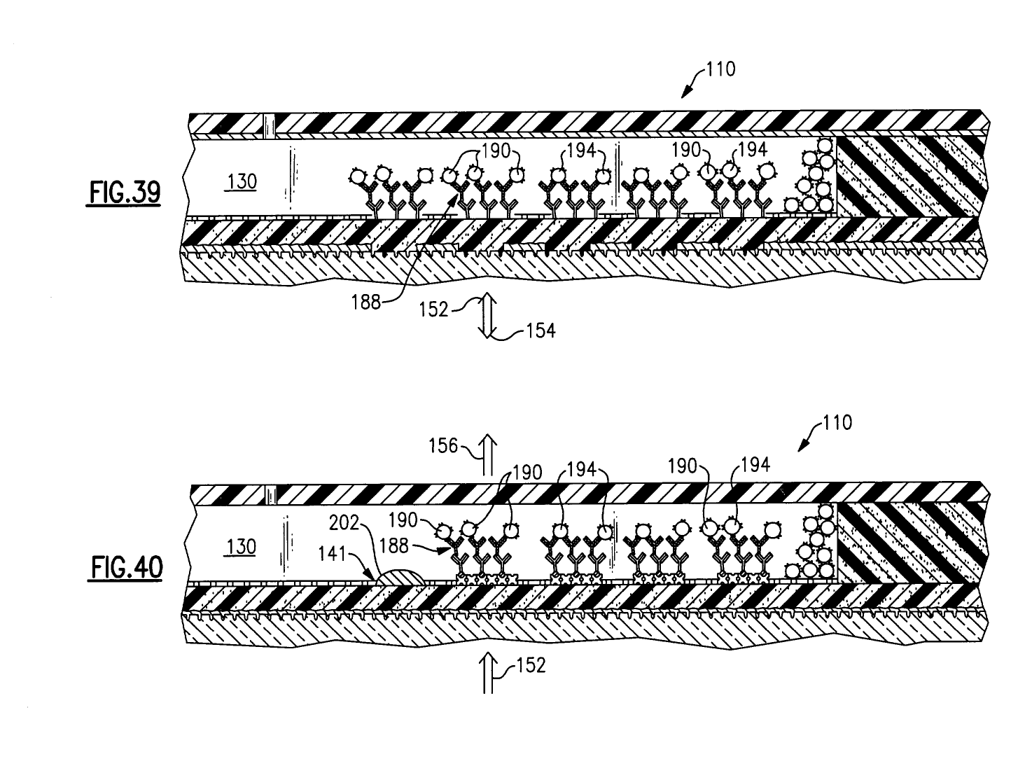

FIG. 40 shows analysis of the same test sample from FIGS. 38A-38C, using the third embodiment of the second implementation of the invention. The flow channels 130 of a bio-disc 110 are flooded with the MNC sample (FIG. 38A). Capillary action, pressure applied with an external applicator, and/or centrifugal force (i.e., the force on a body in curvilinear motion directed away from the center or curvature or axis of rotation) act upon the test sample to achieve contact with the primary antibodies 188. The primary antibodies come into contact with, and capture any CD4⁺ T-cells 190 and CD8⁺ T-cells 194 present within the test sample (FIG. 38B). The optical drive motor 162 (FIG. 10) spins the disc, which clears the capture zones 140 (FIG. 24L) of unbound T-cells. The incident beam 152 of an optical disc drive 112 (FIG. 1) interacts with the captured CD4⁺ T-cells 190 and CD8⁺ T-cells 194 (FIG. 38C) and the transmitted beam 156 is passed to the detector 157 (FIG. 10) for processing and analysis.

Cluster Designation Count—Implementation III

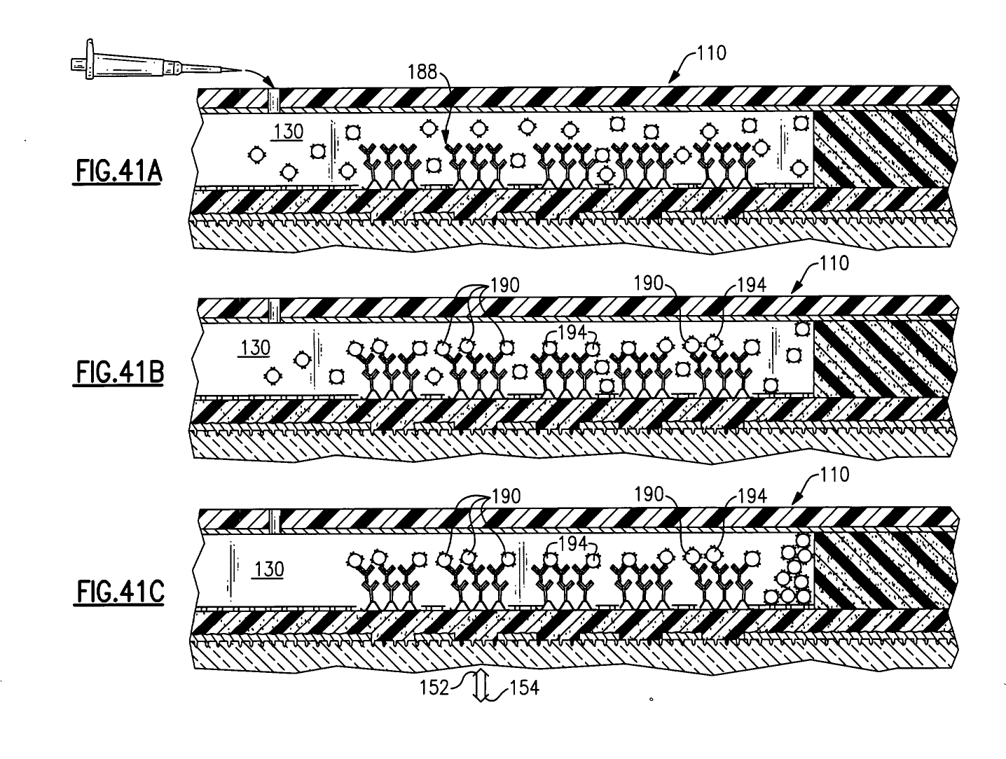

Turning next to FIGS. 41A-41C, analysis of a purified and washed MNC sample (FIG. 17A) using a bio-disc of the third implementation of the invention is shown. The flow channels 130 of a bio-disc 110 are flooded with the MNC sample as illustrated in FIG. 41A. Capillary action, pressure applied with an external applicator, and/or centrifugal force (i.e., the force on a body in curvilinear motion directed away from the center or curvature or axis of rotation) act upon the test sample to achieve contact with the primary antibodies 188. The primary antibodies come into contact with and then capture any CD4⁺ T-cells 190 and CD8⁺ T-cells 194 present within the test sample. This step of the present method is shown FIG. 41B. The optical drive motor 162 (FIG. 10) spins the disc, which clears the capture zones 140 (FIG. 28J) of unbound T-cells. The incident beam 152 of an optical disc drive 112 (FIG. 1) interacts with the captured CD4⁺ T-cells 190 and CD8⁺ T-cells 194, FIG. 41C, and the return beam 154 is reflected to the detector 157 (FIG. 10) for processing and analysis.

FIG. 42 shows analysis of the same test sample from FIGS. 41A-41C, using the second embodiment of the third implementation of the invention. The flow channels 130 of a bio-disc 110 are flooded with the MNC sample (FIG. 41A). Capillary action, pressure applied with an external applicator, and/or centrifugal force (i.e., the force on a body in curvilinear motion directed away from the center or curvature or axis of rotation) act upon the test sample to achieve contact with the primary antibodies 188. The primary antibodies come into contact with, and capture any CD4⁺ T-cells 190 and CD8⁺ T-cells 194 present within the test sample, (FIG. 41B). The optical drive motor 162 (FIG. 10) spins the disc, which clears the capture zones 140 (FIG. 28J) of unbound T-cells. The incident beam 152 of an optical disc drive 112 (FIG. 1) interacts with the captured CD4⁺ T-cells 190 and CD8⁺ T-cells 194 (FIG. 41C) and the return beam 154 is reflected to the detector 157 (FIG. 10) for processing and analysis.

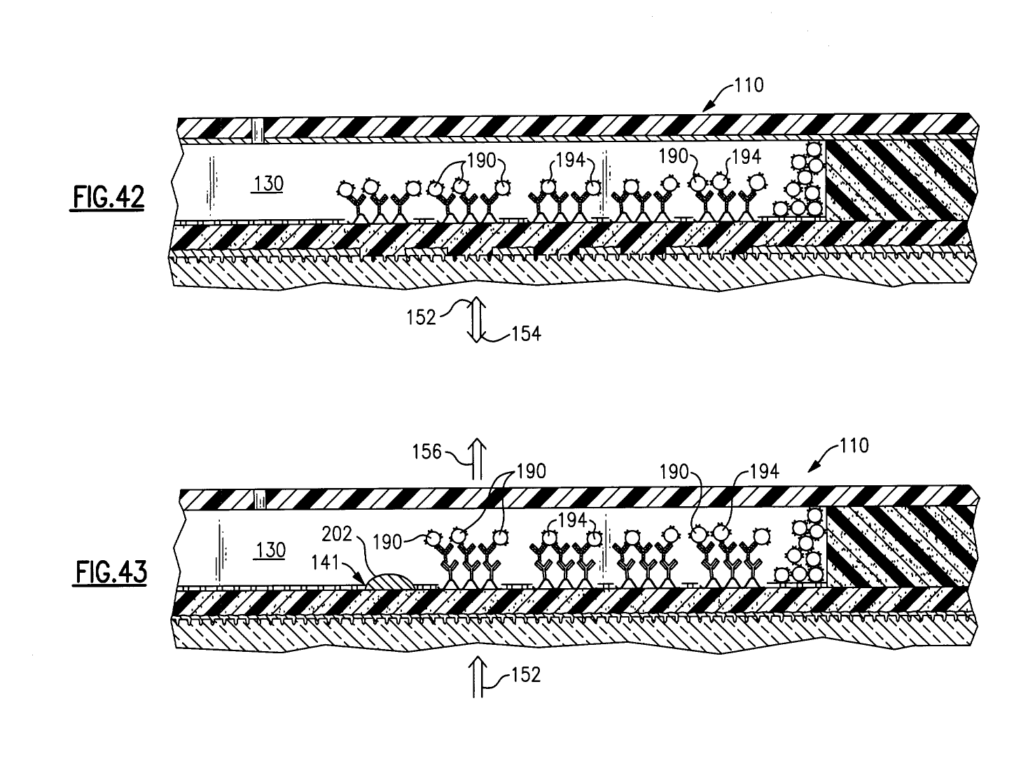

FIG. 43 shows analysis of the same test sample from FIGS. 41A-41C, using the third embodiment of the third implementation of the invention. The flow channels 130 of a bio-disc 110 are flooded with the MNC sample (FIG. 41A). Capillary action, pressure applied with an external applicator, and/or centrifugal force (i.e., the force on a body in curvilinear motion directed away from the center or curvature or axis of rotation) act upon the test sample to achieve contact with the primary antibodies 188. The primary antibodies come into contact with, and capture any CD4⁺ T-cells 190 and CD8⁺ T-cells 194 present within the test sample (FIG. 41B). The optical drive motor 162 (FIG. 10) spins the disc, which clears the capture zones 140 (FIG. 28J) of unbound T-cells. The incident beam 152 of an optical disc drive 112 (FIG. 1) interacts with the captured CD4⁺ T-cells 190 and CD8⁺ T-cells 194 (FIG. 41C) and the transmitted beam 156is passed to the detector 157 (FIG. 10) for processing and analysis.

Cluster Designation Count—Implementation IV

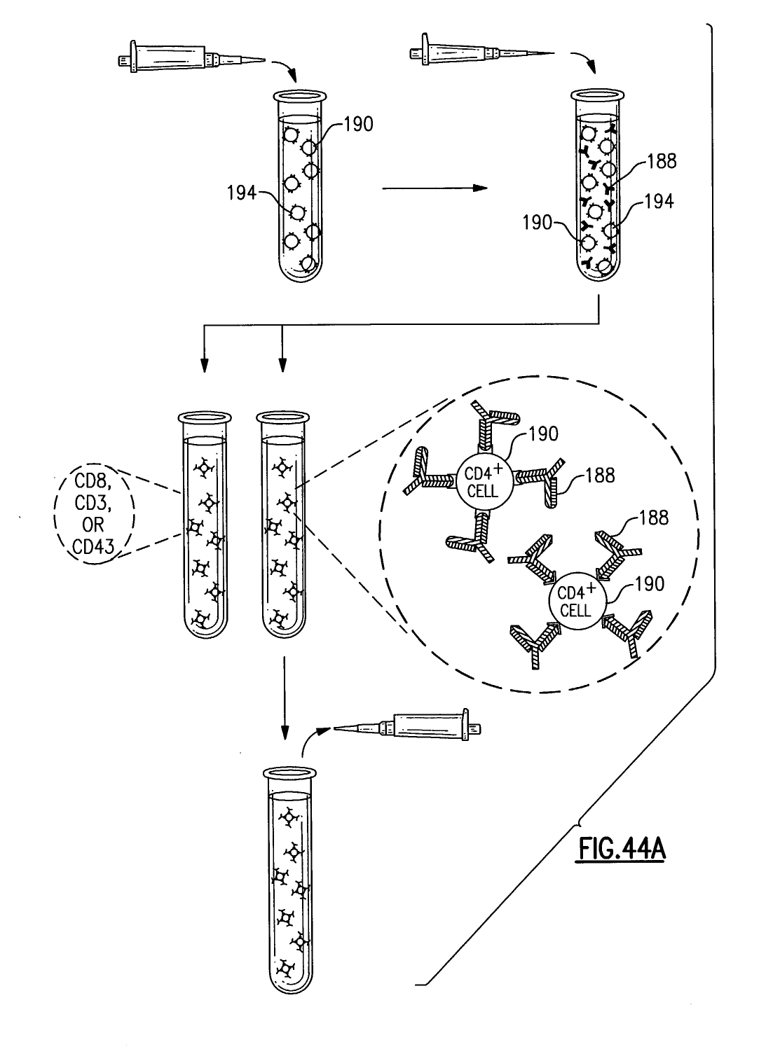

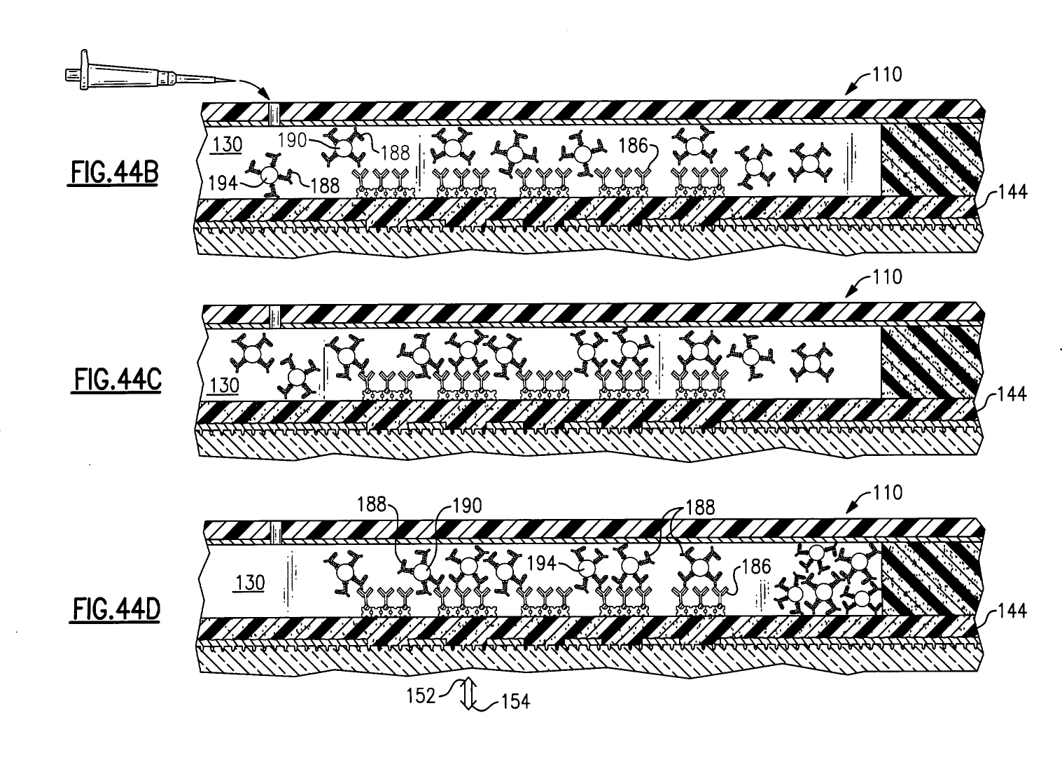

Referring now to FIGS. 44A-44D, analysis of a purified and washed MNC sample (FIG. 17A) using a bio-disc of the fourth implementation of the invention is shown. FIG. 44A is a pictorial flow diagram showing preparation of the primary antibody-T-cell complex. The MNC suspension containing both CD4⁺ T-cells 190 and CD8⁺ T-cells 194 is mixed with primary antibodies 188, and allowed to bind, thereby forming the primary antibody-T-cell complexes. As would be apparent to one of skill in the art, other cells with different surface markers may also be tagged using the current implementation. As shown in FIG. 44B, the flow channels 130 of a bio-disc 110 are flooded with the primary antibody-T-cell complexes. Capillary action, pressure applied with an external applicator, and/or centrifugal force (i.e., the force on a body in curvilinear motion directed away from the center or curvature or axis of rotation) act upon the test sample to achieve contact with the secondary antibodies 188 immobilized on the active layer 144 of the bio-disc 110. The secondary antibodies 186, having an affinity for the primary antibody-T-cell complexes capture the complexes as shown in FIG. 44C. The optical drive motor 162 (FIG. 10) spins the disc, which clears the capture zones 140 (FIG. 32I) of unbound complexes. The incident beam 152 of the optical disc drive 112 (FIG. 1) interacts with the captured complexes, FIG. 44D, and the return beam 154 is reflected to the detector 157 (FIG. 10) for processing and analysis.

FIGS. 44A-44D also show another principal aspect of the methods of this invention. In conjunction with FIG. 17A, there is provided another method of performing a cluster designation count. This method includes the steps of (1) providing a blood sample in a tube containing a separation gradient, (2) rotating the tube at a time and speed sufficient to separate the blood sample into layers, (3) resuspending a MNC layer containing T-cells to form a MNC suspension, (4) adding a primary antibody to the MNC suspension to form a primary antibody-T-cell complex, (5) providing a sample of the primary antibody-T-cell complex on a disc surface that includes at least one capture zone containing at least one capture agent, (6) loading the disc into an optical reader, (7) directing an incident beam of electromagnetic radiation to the capture zone, (8) detecting a beam of electromagnetic radiation formed after interacting with the disc at the capture zone, (9) converting the detected beam into an output signal, and (10) analyzing the output signal to extract information relating to the number of cells captured at the capture zone. In one embodiment of this method, the optical disc is constructed with a reflective layer such that light directed to the capture zone and not striking a cell is reflected. In another embodiment of this method, the optical disc is constructed such that light directed to the capture zone and not striking a cell is transmitted through the optical disc.

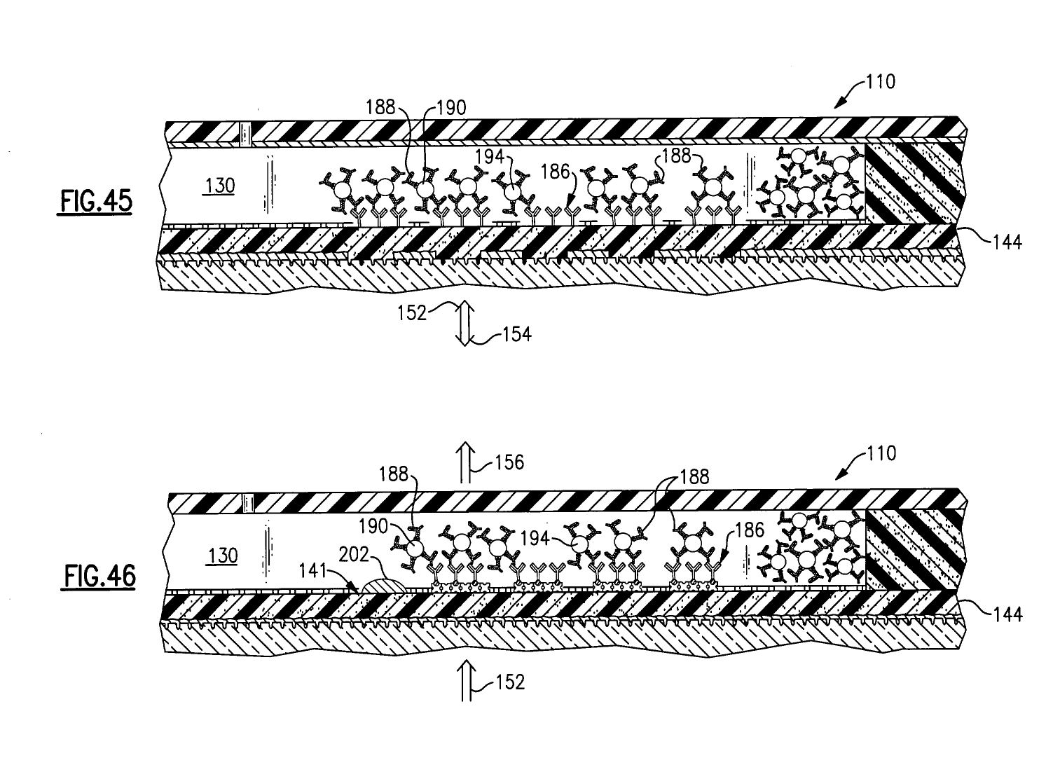

FIG. 45 shows analysis of the same test sample from FIGS. 44A-44D, using the second embodiment of the fourth implementation of the invention. The flow channels 130 of a bio-disc 110 are flooded with the primary antibody-T-cell complexes (FIG. 44B). Capillary action, pressure applied with an external applicator, and/or centrifugal force (i.e., the force on a body in curvilinear motion directed away from the center or curvature or axis of rotation) act upon the test sample to achieve contact with the secondary antibodies 188 immobilized on the active layer 144 of the bio-disc 110. The secondary antibodies 186, having an affinity for the primary antibody-T-cell complexes capture the complexes (FIG. 44C). The optical drive motor 162 (FIG. 10) spins the disc, which clears the capture zones 140 (FIG. 32I) of unbound complexes. The incident beam 152 of the optical disc drive 112 (FIG. 1) interacts with the captured complexes (FIG. 44D) and the return beam 154 is reflected to the detector 157 (FIG. 10) for processing and analysis.

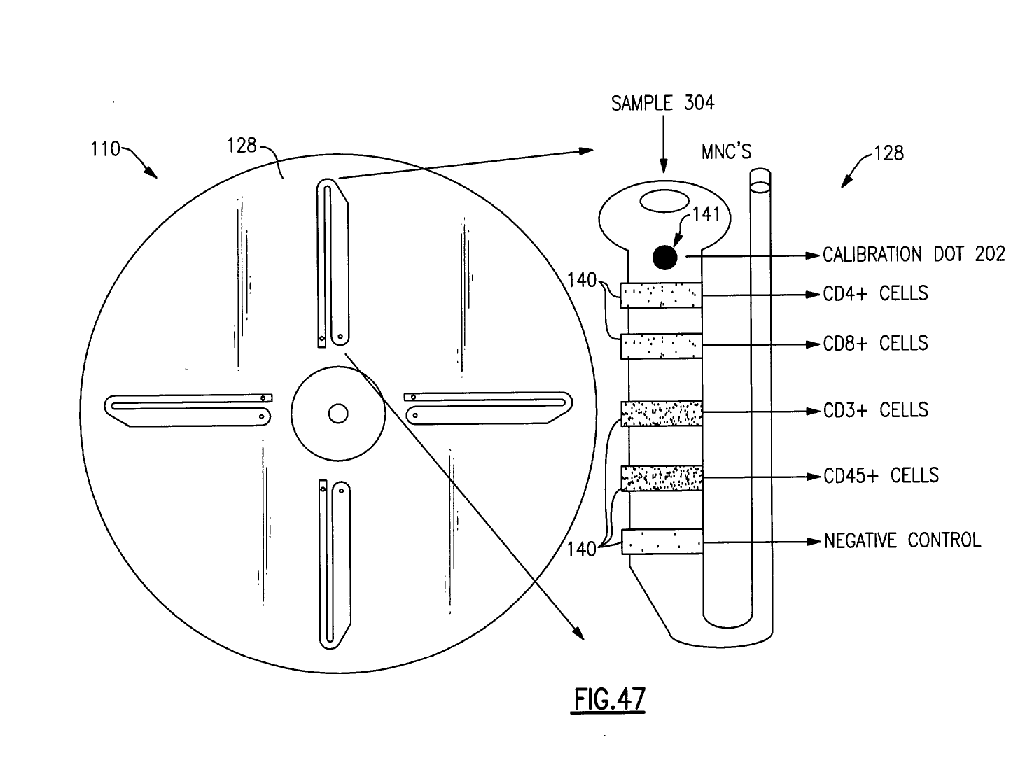

FIG. 46 shows analysis of the same test sample from FIGS. 44A-44D, using the third embodiment of the fourth implementation of the invention. The flow channels 130 of a bio-disc 110 are flooded with the primary antibody-T-cell complexes (FIG. 44B). Capillary action, pressure applied with an external applicator, and/or centrifugal force (i.e., the force on a body in curvilinear motion directed away from the center or curvature or axis of rotation) act upon the test sample to achieve contact with the secondary antibodies 188 immobilized on the active layer 144 of the bio-disc 110. The secondary antibodies 186, having an affinity for the primary antibody-T-cell complexes capture the complexes (FIG. 44C). The optical drive motor 162 (FIG. 10) spins the disc, which clears the capture zones 140 (FIG. 32I) of unbound complexes. The incident beam 152 of an optical disc drive 112 (FIG. 1) interacts with the captured complexes (FIG. 44D) and the transmitted beam 156 is passed to the detector 157 (FIG. 10) for processing and analysis.

Those skilled in the art will appreciate in light of these teachings that one or more embodiments of one or more implementations of the methods of the invention may be combined without departing from the scope and spirit of this invention.

Cell Detection and Related Software

Referring now to FIG. 47, there is shown the optical bio-disc 110 including a fluid circuit 128 for holding a sample. FIG. 47 also shows the fluidic circuit 47 enlarged to illustrate different capture or target zones 140 and the target zone 141 including the reference mark or calibration dot 202. In this specific embodiment, five capture zones 140 are employed, each respectively implemented to capture CD4+ cells, CD8+ cells, CD3+ cells, CD45+ cells, and myoglobin as the negative control.

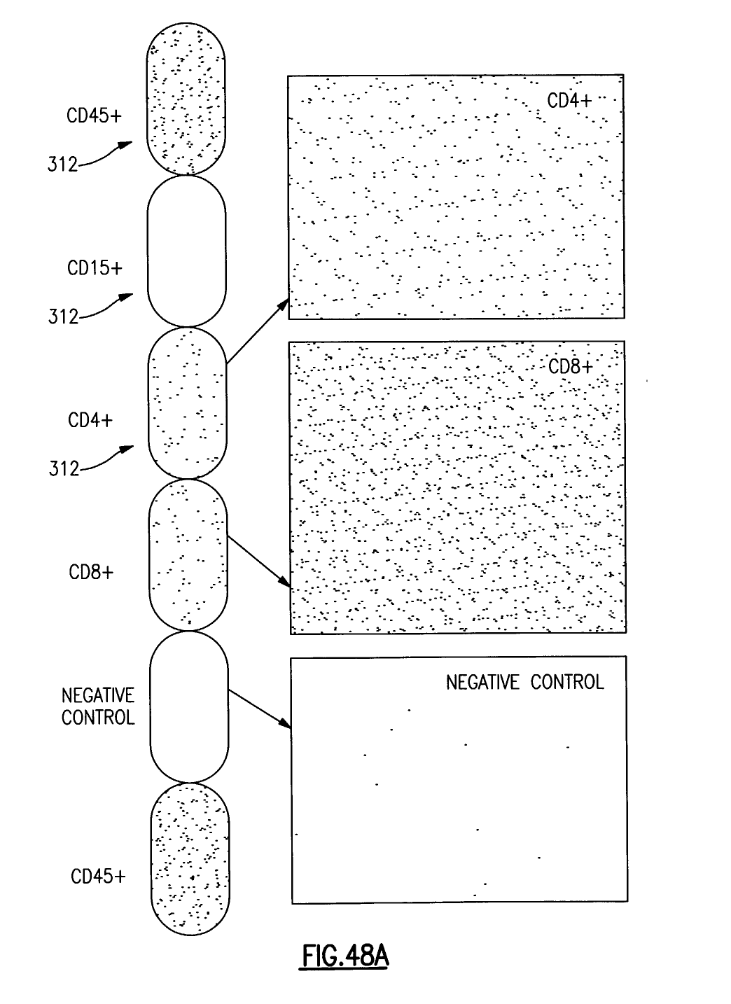

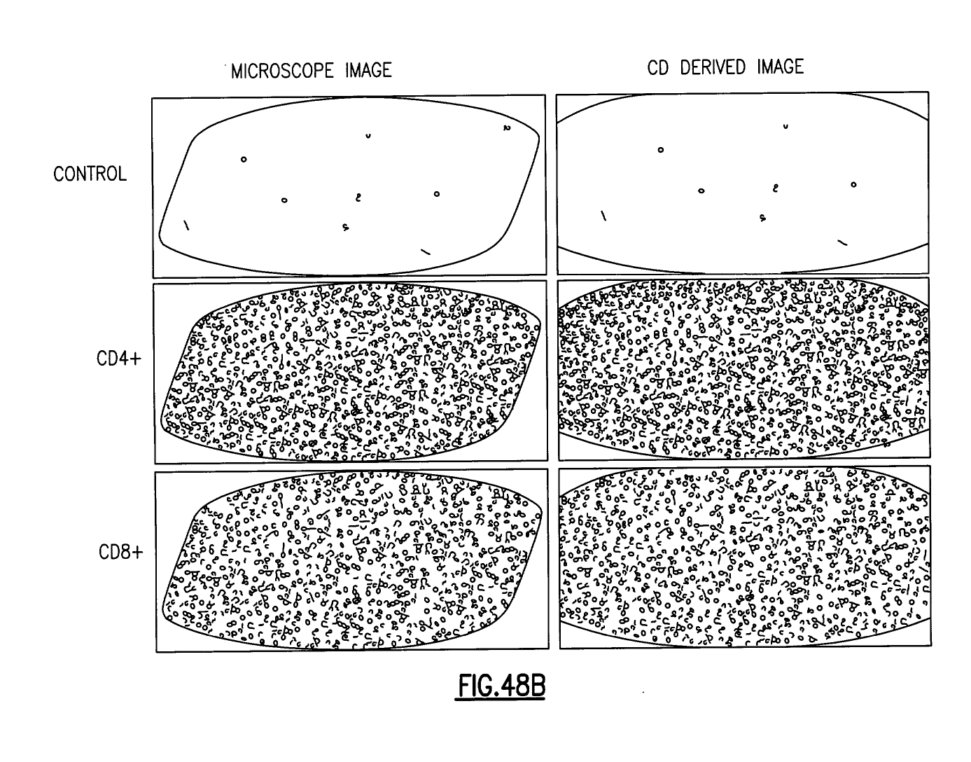

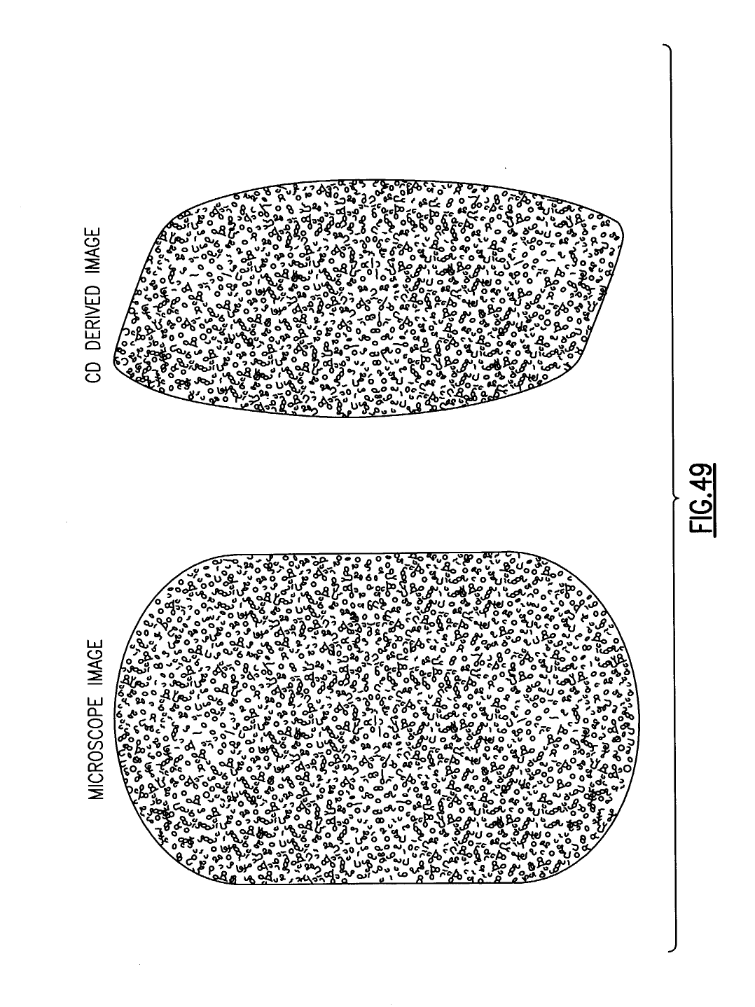

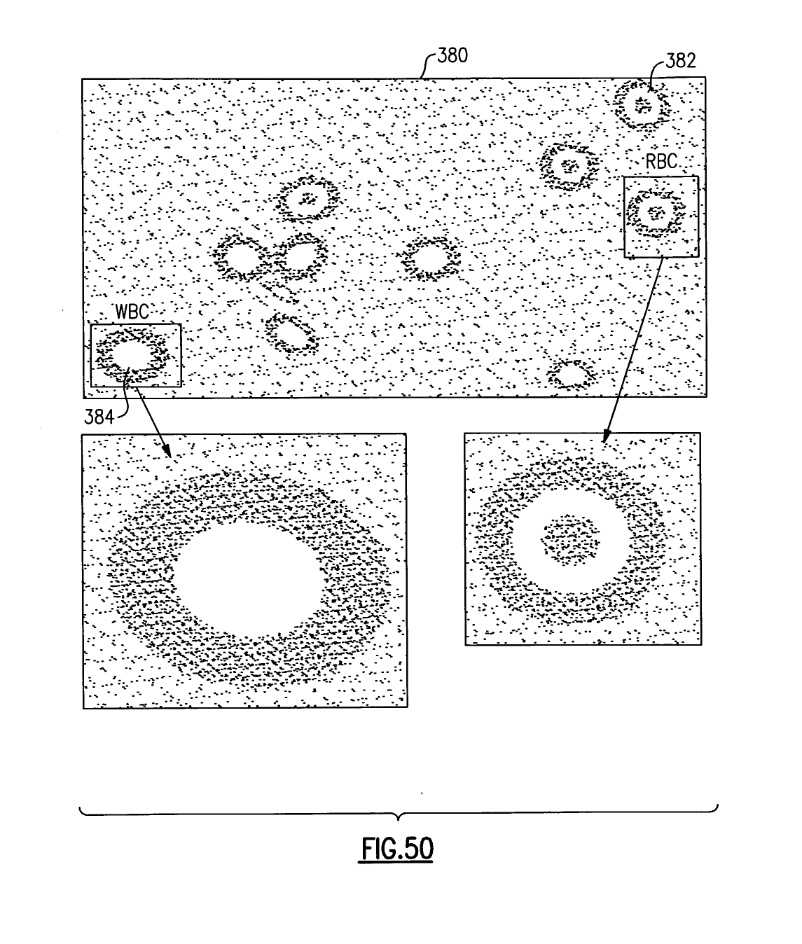

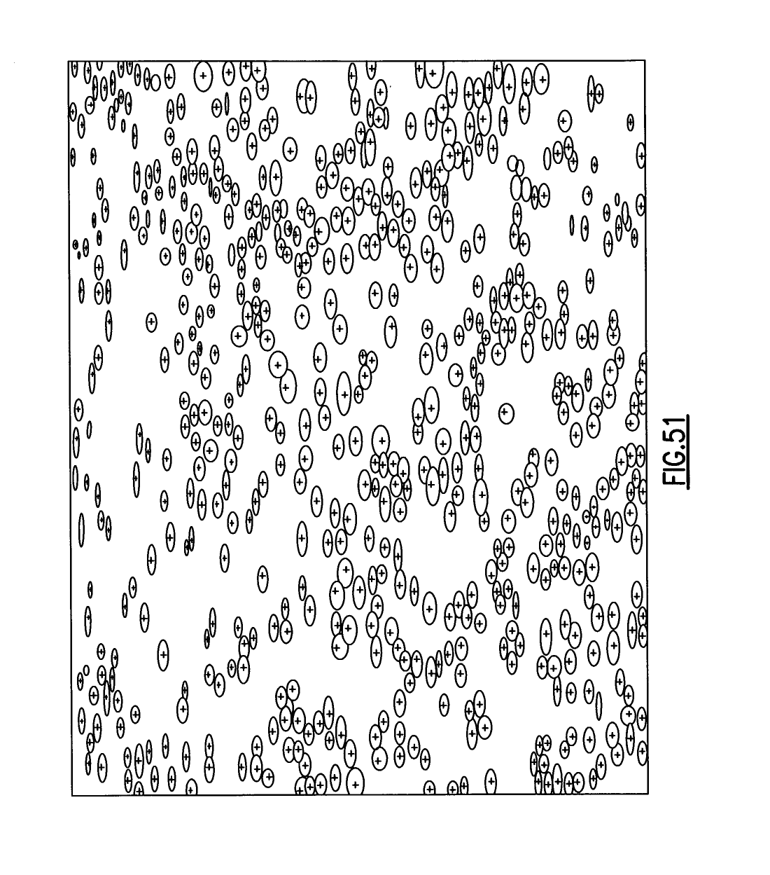

FIG. 48A an image obtained from a different embodiment including six capture zones 140 each implemented, respectively, to capture CD45+ cells, CD15+ cells, CD4+ cells CD8+ cells, a second capture zone for CD15+ cells, and a second capture zone for CD45+ cells. In this embodiment, the two CD45 and CD15 capture zones may be used as a validation control or check to verify that the capture efficiency and expected count results are in agreement. FIG. 48A also shows a series of cell surface antigens with enlarged views for CD4, CD8, and a control. As indicated here, the image is of a number of cells shown against a background field. FIG. 48B represents a close up view of the control, CD4, and CD8 capture zones from an actual microscope image compared to a bio-disc derived image according to the present invention. FIG. 49 illustrates another comparison in greater detail of an actual microscope image and a corresponding bio-disc image according to the present invention. As represented in FIGS. 48B and 49, the inventors have found that the bio-disc images are of equal quality and resolution compared to those obtainable from a microscope. These images thus demonstrate that individual cells can be made visible against a background using the apparatus and methods of the present invention. Methods for detecting investigational features are described in more detail in commonly assigned U.S. Provisional Application Serial Nos. 60/270,095 and 60/292,108 each entitled “Signal Processing Apparatus and Methods for Obtaining Signal Signatures of Investigational Features Detected on a Surface of an Optical Disc Assembly” respectively filed on Feb. 2, 2001 and May 18, 2001, and in commonly assigned U.S. patent application Ser. No. 10/043,688 entitled “Optical Disc Analysis System Including Related Methods For Biological and Medical Imaging” as filed on Jan. 10, 2002, all of which are incorporated herein by reference.

These cells can be detected through one of a variety of different methods including, for example, using edge detection hardware or software to detect and count sufficiently large changes in the level of transmitted or reflected light and thus count the transitions and hence the cells. Another method, described in more detail below, uses image or pattern recognition software to identify cells against the background. Image recognition can distinguish WBCs from RBCs, and also distinguish neutrophils, monocytes, basophils, eosinophils, granulocytes, and lymphocytes.

An optical disc with tracks on the order of 1.6 microns apart can be used to image cells or aggregates on the disc. For example, a white blood cell would typically have a diameter of at least 5 and as many as 12 tracks, and therefore an image of that white blood cell can be obtained.

To obtain such an image, a transmissive disc of the type shown in FIGS. 2, 3 and 4 may be used (although a reflective disc would be operable), and a disc drive system of the type shown in FIG. 10 including trigger sensor 160 and top detector 158. Trigger detector 158 detects a trigger mark 126 in a transmissive disc and provides a signal to a computer that data is to be collected and/or processed when that mark is detected. As the light source passes across the tracks in the viewing window, an image is obtained for the received transmitted light. The top detector in this case can be a single detector, or an array of multiple detector elements oriented in the radial and/or circumferential direction. Such detectors and detection methods are described, for example, in commonly assigned U.S. Provisional Application Serial No. 60/247,465 entitled “Optical Disc Drive For Bio-Optical Disc” filed Nov. 9, 2000; U.S. Provisional Application Serial No. 60/293,093 entitled “Disc Drive Assembly For Optical Bio-Discs” filed May 22, 2001; and U.S. patent application Ser. No. 10/008,156 entitled “Disc Drive System and Methods for Use with Bio-discs” filed Nov. 9, 2001, each of which is incorporated herein by reference in its entirety.

After images such as those in FIGS. 48A, 48B and 49 are obtained, the image data can be processed further with image recognition software designed to identify desired features. It is further desirable that the image recognition software not only have the ability to distinguish cells from background, but also one type of cell from another.

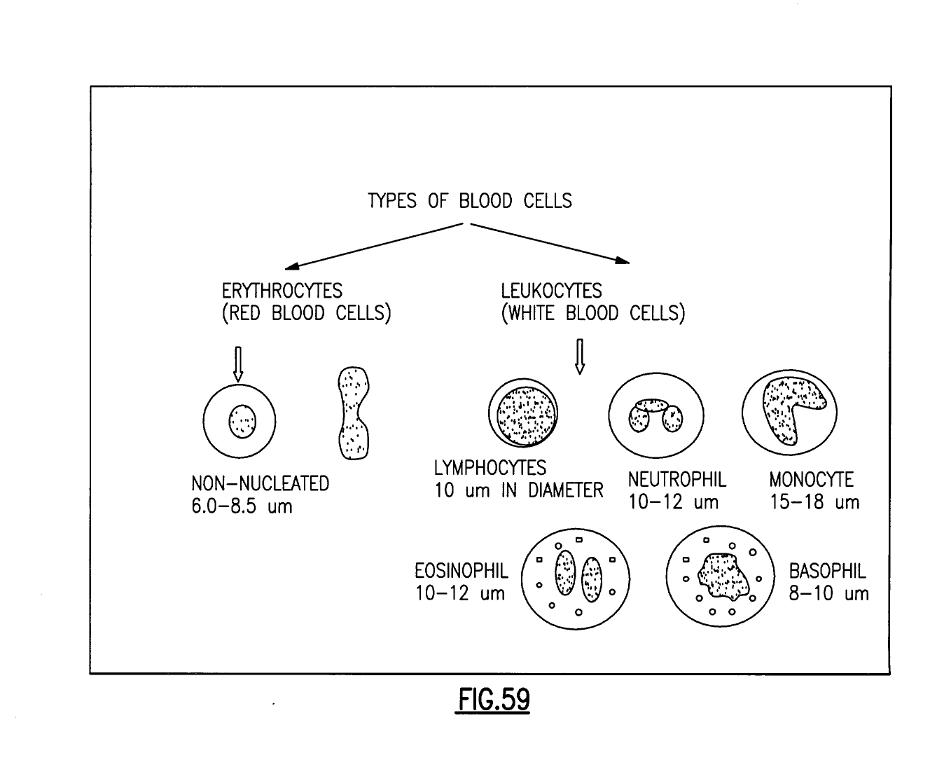

With reference now to FIG. 50, there is shown an image derived from investigational data that includes both red blood cells and white blood cells. As indicated in the enlarged views, these white and red cells have clearly distinct characteristics and thus can be detected against the background and can also be distinguished from each other with image recognition. In addition, it is also possible to distinguish types of white blood cells from each other, including lymphocytes, monocytes, neutrophils, eosinophils, granulocytes, and basophils by staining the nuclei of these cells as discussed below in conjunction with FIG. 59, 60, and 61.

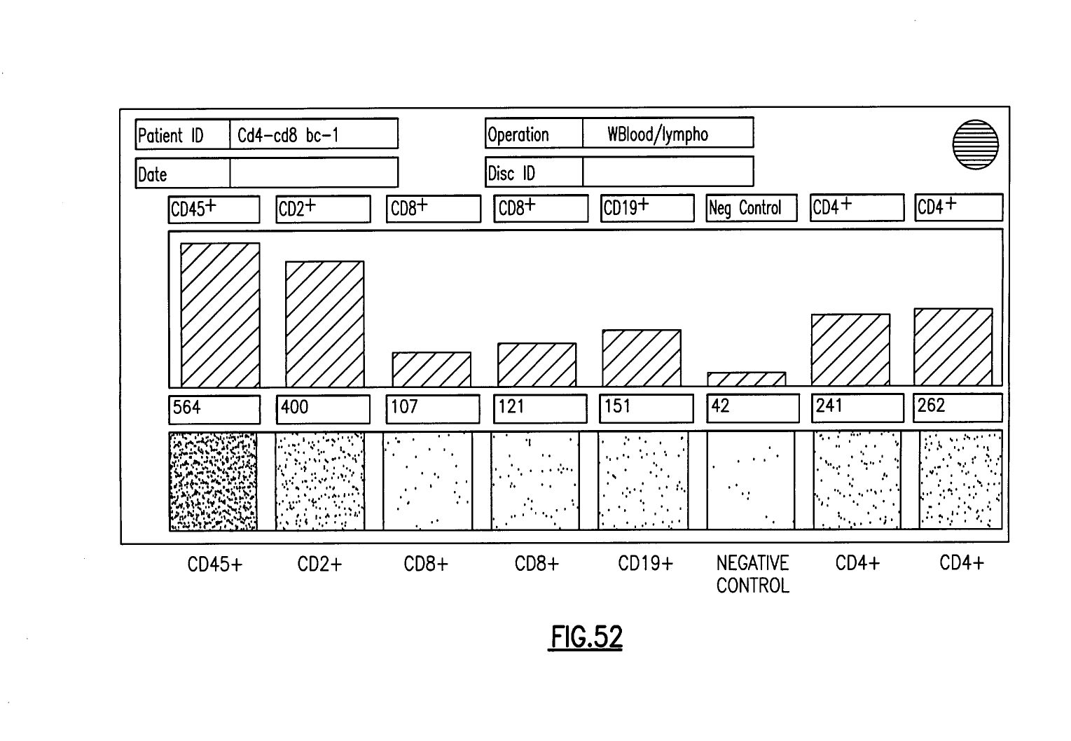

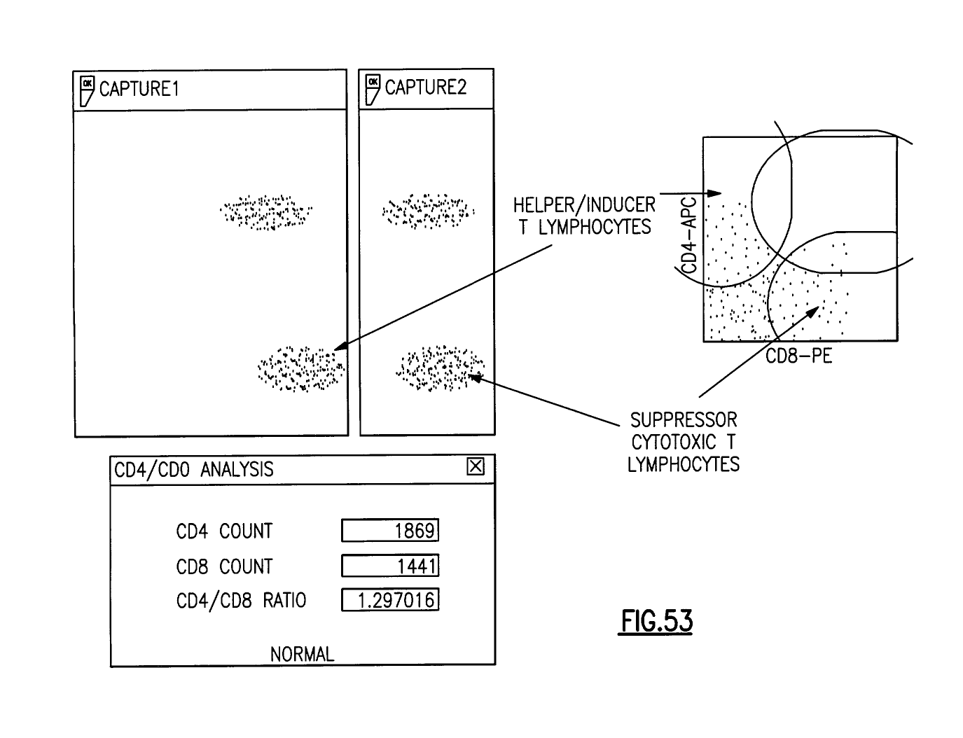

FIG. 51 shows a sample field with a number of cells with a plus sign indicating each object that is identified as a cell. After the number of cells have been detected for every zone or any number of desired zones, resulting cell count data can be displayed in a single screen that provides an easy to view representation such as that shown in FIG. 52. As depicted in FIG. 52, the specific cell counts are provided along with a bar graph to demonstrate relative numbers of cells. In the case of a CD4/CD8 analysis, the system can also produce a CD4/CD8 ratio as well as any other desired mathematical calculation or comparison. FIG. 53 provides a different view of the process showing cells in an image field being converted to a CD4 count, a CD8 count, and a ratio, with the output indicating that the ratio is in a normal range.

Aspects of the present invention relating to cell detection and associated processing methods are also disclosed in U.S. Provisional Application Serial No. 60/316,273 entitled “Capture Layer Assemblies and Optical Bio-Discs for Immunophenotyping” filed Aug. 31, 2001; U.S. Provisional Application Serial No. 60/318,026 entitled “Methods for Imaging Blood cells, Blood-Borne Parasites and Pathogens, and Other Biological Matter Including Related Optical Bio-Discs and Drive Assemblies” filed Sep. 7, 2001; U.S. Provisional Application Serial No. 60/322,527 entitled “Optical Analysis Discs Including Microfluidic Circuits for Performing Cell Counts” filed Sep. 14, 2001; U.S. Provisional Application Serial No. 60/322,040 entitled “Optical Analysis Discs Including Fluidic Circuits for Optical Imaging and Quantitative Evaluation of Blood Cells Including Lymphocytes” filed Sep. 11, 2001; U.S. Provisional Application Serial No. 60/322,863 entitled “Methods for Differential Cell Counts Including Leukocytes and Use of Optical Bio-Disc for Performing Same” filed Sep. 12, 2001; and U.S. Provisional Application Serial No. 60/322,793 entitled “Methods for Reducing Non-Specific Binding of Cells on Optical Bio-Discs Utilizing Charged Matter Including Heparin, Plasma, or Poly-Lysine” filed Sep. 17, 2001, all of which are herein incorporated by reference.

Differential Cell Count Methods and Related Software

A number of methods and related algorithms for white blood cell counting using optical disc data are herein discussed in further detail. These methods and related algorithms are not limited to counting white blood cells, but may be readily applied to conducting counts of any type of cellular matter including, but not limited to, red blood cells, white blood cells, beads, and any other objects, both biological and non-biological, that produce similar optical signatures that can be detected by an optical reader.

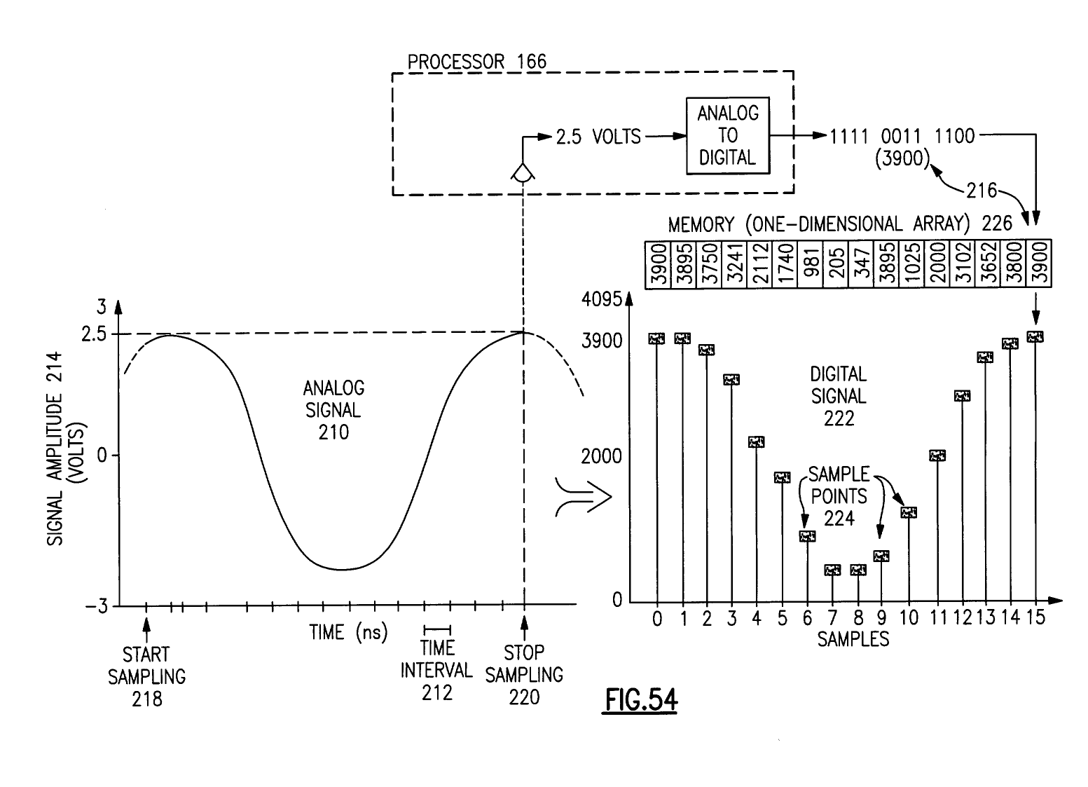

For the purposes of illustration, the following description of the methods and algorithms related to the present invention as described with reference to FIGS. 54-58, are directed to white blood cell counting. With some modifications, these methods and algorithms can be applied to counting other types of cells, or objects similar in size to white blood cells. The data evaluation aspects of the cell counting methods and algorithms are described generally herein to provide related background for the methods and apparatus of the present invention. Methods and algorithms for capturing and processing investigational data from the optical bio-disc has general broad applicability and has been disclosed in further detail in commonly assigned U.S. Provisional Application No. 60/291,233 entitled “Variable Sampling Control For Rendering Pixelation of Analysis Results In Optical Bio-Disc Assembly And Apparatus Relating Thereto” filed May 16, 2001 which is herein incorporated by reference and the above incorporated U.S. Provisional Application No. 60/404,921 entitled “Methods For Differential Cell Counts Including Related Apparatus And Software For Performing Same”. In the following discussion, the basic scheme of the methods and algorithms with a brief explanation is presented. As illustrated in FIG. 10, information concerning attributes of the biological test sample is retrieved from the optical bio-disc 110 in the form of a beam of electromagnetic radiation that has been modified or modulated by interaction with the test sample. In the case of the reflective optical bio-disc discussed in conjunction with FIGS. 2, 3, 4, 11, 13, and 15, the return beam 154 carries the information about the biological sample. As discussed above, such information about the biological sample is contained in the return beam essentially only when the incident beam is within the flow channel 130 or target zones 140 and thus in contact with the sample. In the reflective embodiment of the bio-disc 110, the return beam 154 may also carry information encoded in or on the reflective layer 142 or otherwise encoded in the wobble grooves 170 illustrated in FIGS. 13 and 14. As would be apparent to one of skill in the art, pre-recorded information is contained in the return beam 154 of the reflective disc with target zones, only when the corresponding incident beam is in contact with the reflective layer 142. Such information is not contained in the return beam 154 when the incident beam 152 is in an area where the information bearing reflective layer 142 has been removed or is otherwise absent. In the case of the transmissive optical bio-disc discussed in conjunction with FIGS. 5, 6, 8, 9, 12, 14, and 16, the transmitted beam 156 carries the information about the biological sample.

With continuing reference to FIG. 10, the information about the biological test sample, whether it is obtained from the return beam 154 of the reflective disc or the transmitted beam 156 of the transmissive disc, is directed to a processor 166 for signal processing. This processing involves transformation of the analog signal detected by the bottom detector 157 (reflective disc) or the top detector 158 (transmissive disc) to a discrete digital form.

Referring next to FIG. 54, the signal transformation involves sampling the analog signal 210 at fixed time intervals 212, and encoding the corresponding instantaneous analog amplitude 214 of the signal as a discrete binary integer 216. Sampling is started at some start time 218 and stopped at some end time 220. The two common values associated with any analog-to-digital conversion process are sampling frequency and bit depth. The sampling frequency, also called the sampling rate, is the number of samples taken per unit time. A higher sampling frequency yields a smaller time interval 212 between consecutive samples, which results in a higher fidelity of the digital signal 222 compared to the original analog signal 210. Bit depth is the number of bits used in each sample point to encode the sampled amplitude 214 of the analog signal 210. The greater the bit depth, the better the binary integer 216 will approximate the original analog amplitude 214. In the present embodiment, the sampling rate is 8 MHz with a bit depth of 12 bits per sample, allowing an integer sample range of 0 to 4095 (0 to (2ⁿ⁻1), where n is the bit depth. This combination may change to accommodate the particular accuracy necessary in other embodiments. By way of example and not limitation, it may be desirable to increase sampling frequency in embodiments involving methods for counting beads, which are generally smaller than cells. The sampled data is then sent to processor 166 for analog-to-digital transformation.

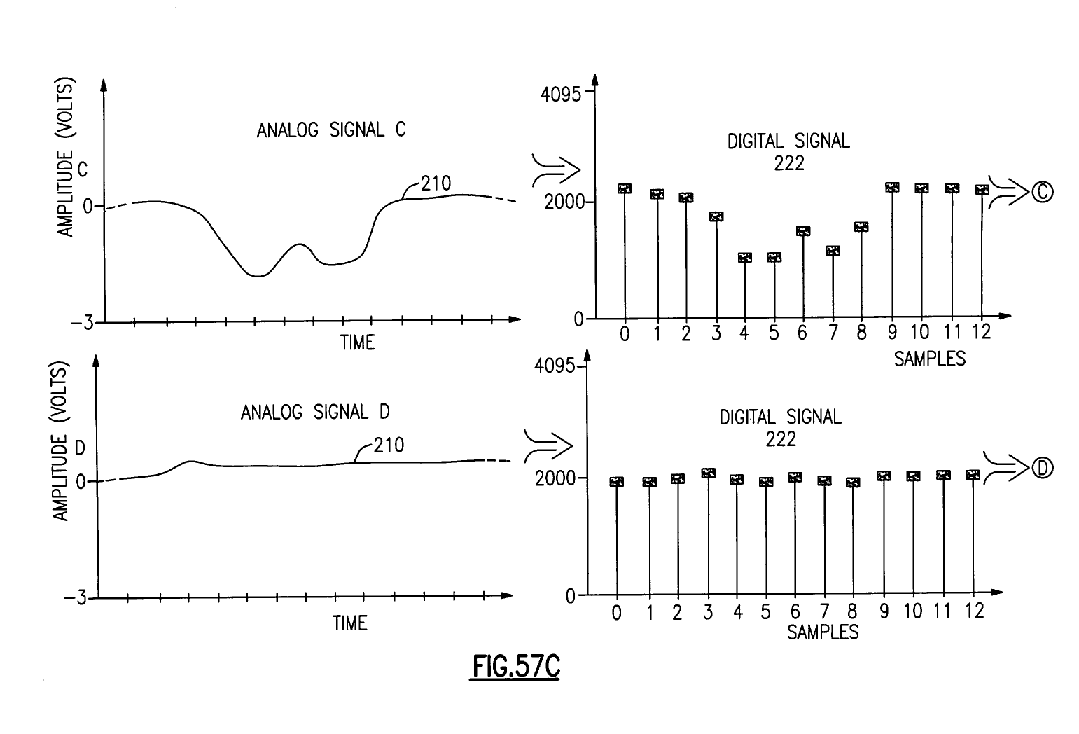

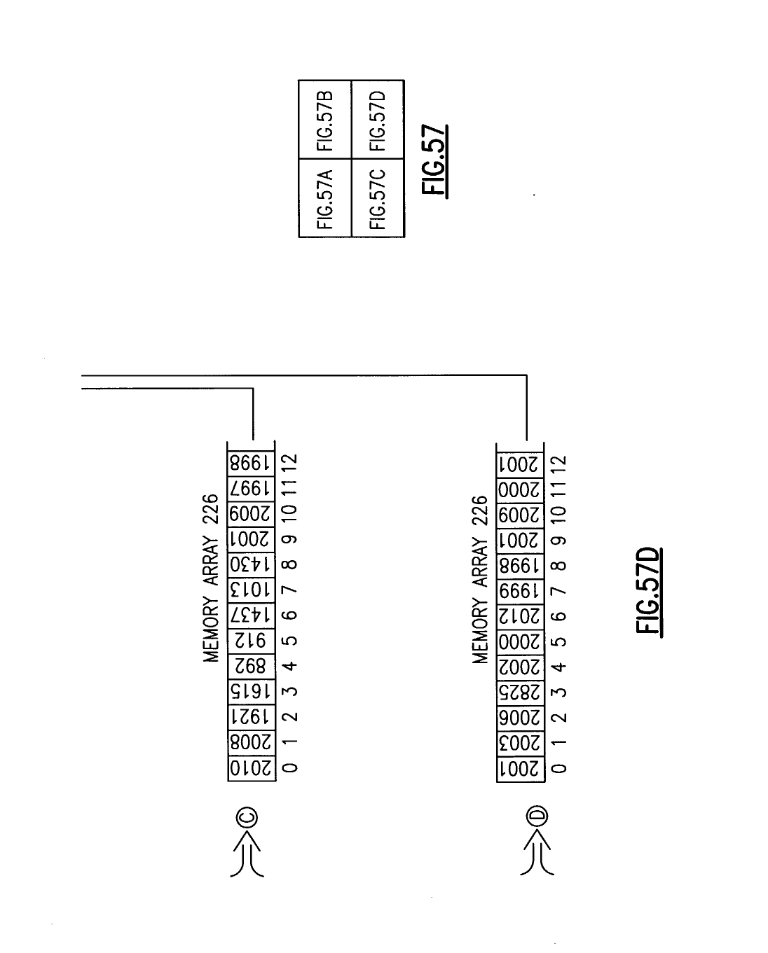

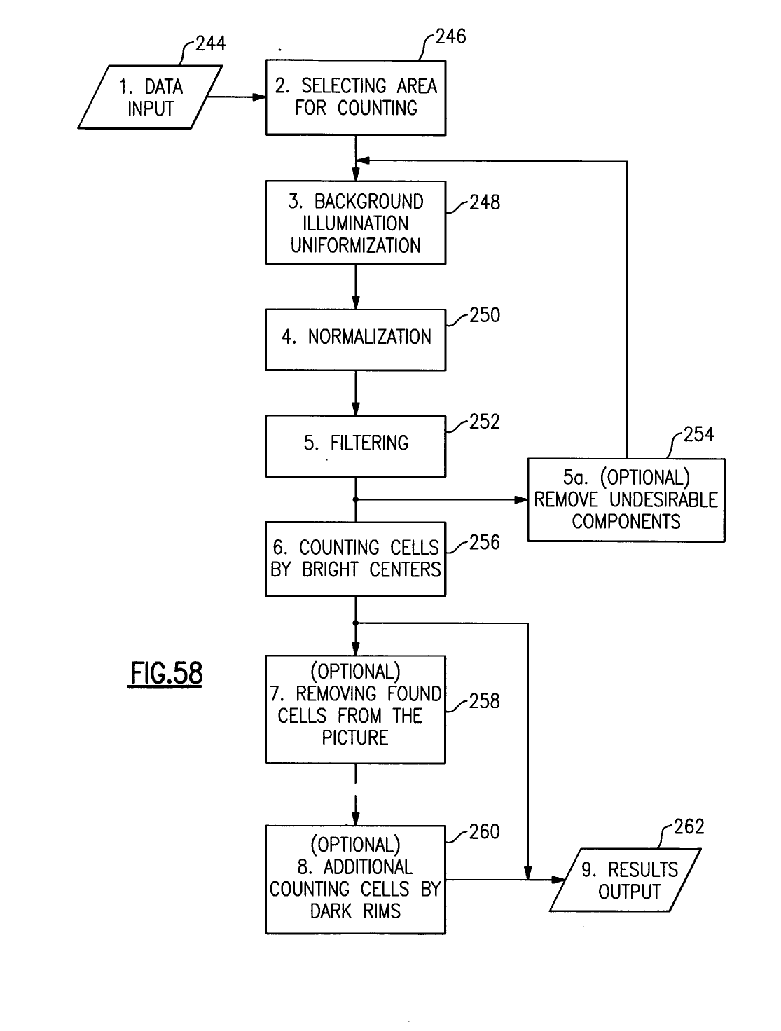

During the analog-to-digital transformation, each consecutive sample point 224 along the laser path is stored consecutively on disc or in memory as a one-dimensional array 226. Each consecutive track contributes an independent one-dimensional array, which yields a two-dimensional array 228 (FIG. 57A) that is analogous to an image.

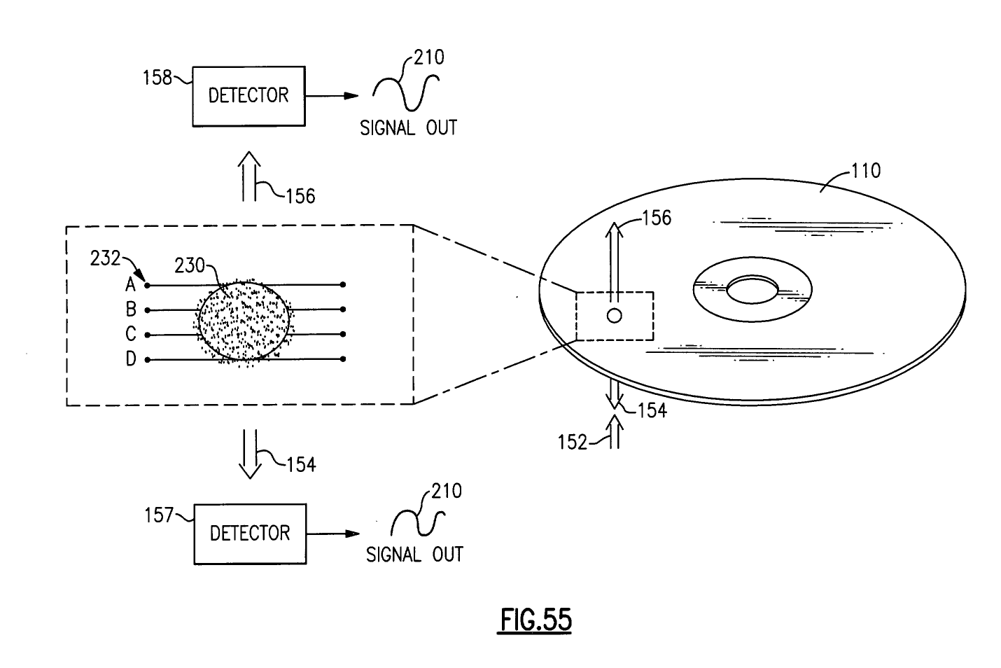

FIG. 55 is a perspective view of an optical bio-disc 110 of the present invention with an enlarged detailed perspective view of the section indicated showing a captured white blood cell 230 positioned relative to the tracks 232 of the optical bio-disc. As shown, the interaction of incident beam 152 with white blood cell 230 yields a signal-containing beam, either in the form of a return beam 154 of the reflective disc or a transmitted beam 156 of the transmissive disc, which is detected by either of detectors 157 or 158.

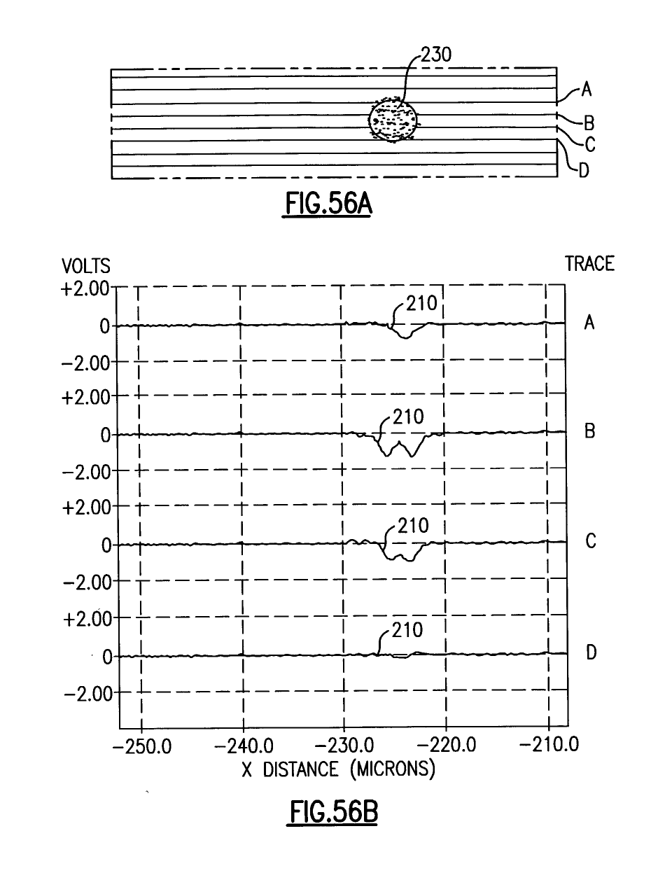

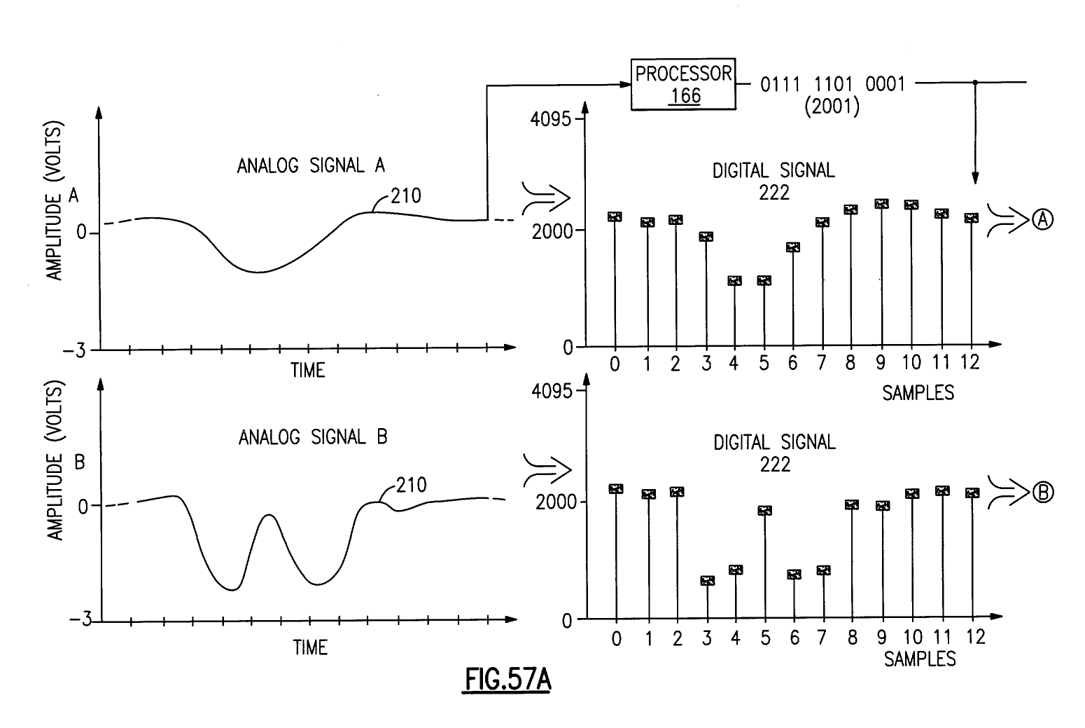

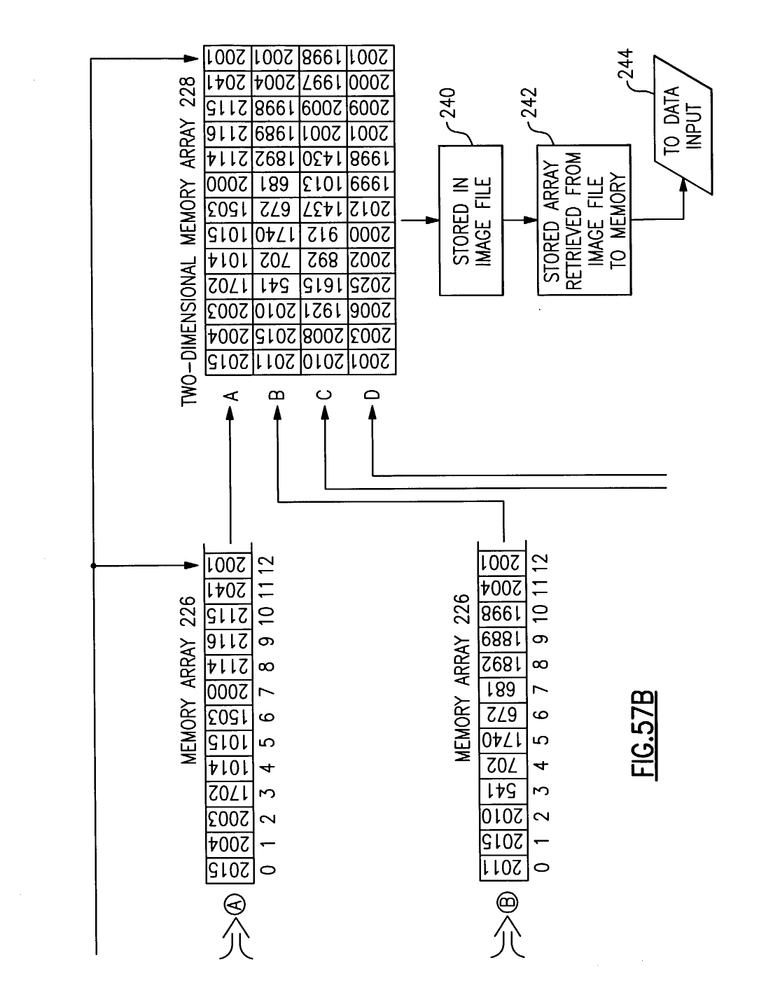

FIG. 56A is another graphical representation of the white blood cell 230 positioned relative to the tracks 232 of the optical bio-disc 110 shown in FIG. 55. As shown in FIGS. 55 and 56A, the white blood cell 230 covers approximately four tracks A, B, C, and D. FIG. 56B shows a series of signature traces derived from the white blood cell 210 of FIGS. 55 and 56A. As indicated in FIG. 56B, the detection system provides four analogue signals A, B, C, and D corresponding to tracks A, B, C, and D. As further shown in FIG. 56B, each of the analogue signals A, B, C, and D carries specific information about the white blood cell 230. Thus as illustrated, a scan over a white blood cell 230 yields distinct perturbations of the incident beam that can be detected and processed. The analog signature traces (signals) 210 are then directed to processor 166 for transformation to an analogous digital signal 222 as shown in FIGS. 57A and 57C as discussed in further detail below.

FIG. 57 is a graphical representation illustrating the relationship between FIGS. 57A, 57B, 57C, and 57D. FIGS. 57A, 57B, 57C, and 57D are pictorial graphical representations of transformation of the signature traces from FIG. 56B into digital signals 222 that are stored as one-dimensional arrays 226 and combined into a two-dimensional array 228 for data input 244.

With particular reference now to FIG. 57A, there is shown sampled analog signals 210 from tracks A and B of the optical bio-disc shown in FIGS. 55 and 56A. Processor 166 then encodes the corresponding instantaneous analog amplitude 214 of the analog signal 210 as a discrete binary integer 216 (see FIG. 54). The resulting series of data points is the digital signal 222 that is analogous to the sampled analog signal 210.

Referring next to FIG. 57B, digital signal 222 from tracks A and B (FIG. 57A) is stored as an independent one-dimensional memory array 226. Each consecutive track contributes a corresponding one-dimensional array, which when combined with the previous one-dimensional arrays, yields a two-dimensional array 228 that is analogous to an image. The digital data is then stored in memory or on disc as a two-dimensional array 228 of sample points 224 (FIG. 54) that represent the relative intensity of the return beam 154 or transmitted beam 156 (FIG. 55) at a particular point in the sample area. The two-dimensional array is then stored in memory or on disc in the form of a raw file or image file 240 as represented in FIG. 57B. The data stored in the image file 240 is then retrieved 242 to memory and used as data input 244 to analyzer 168 shown in FIG. 10.

FIG. 57C shows sampled analog signals 210 from tracks C and D of the optical bio-disc shown in FIGS. 55 and 56A. Processor 166 then encodes the corresponding instantaneous analog amplitude 214 of the analog signal 210 as a discrete binary integer 216 (FIG. 54). The resulting series of data points is the digital signal 222 that is analogous to the sampled analog signal 210.

Referring now to FIG. 57D, digital signal 222 from tracks C and D is stored as an independent one-dimensional memory array 226. Each consecutive track contributes a corresponding one-dimensional array, which when combined with the previous one-dimensional arrays, yields a two-dimensional array 228 that is analogous to an image. As above, the digital data is then stored in memory or on disc as a two-dimensional array 228 of sample points 224 (FIG. 54) that represent the relative intensity of the return beam 154 or transmitted beam 156 (FIG. 55) at a particular point in the sample area. The two-dimensional array is then stored in memory or on disc in the form of a raw file or image file 240 as shown in FIG. 57B. As indicated above, the data stored in the image file 240 is then retrieved 242 to memory and used as data input 244 to analyzer 168 FIG. 10.

The computational and processing algorithms of the present invention are stored in analyzer 168 (FIG. 10) and applied to the input data 244 to produce useful output results 262 (FIG. 58) that may be displayed on the display monitor 114 (FIG. 10). With reference now to FIG. 58 there is shown a logic flow chart of the principal steps for data evaluation according to the processing methods and computational algorithms of the present invention. A first principal step of the present processing method involves receipt of the input data 244. As described above, data evaluation starts with an array of integers in the range of 0 to 4096.

The next principle step 246 is selecting an area of the disc for counting. Once this area is defined, an objective then becomes making an actual count of all white blood cells contained in the defined area. The implementation of step 246 depends on the configuration of the disc and user's options. By way of example and not limitation, embodiments of the invention using discs with windows such as the target zones 140 shown in FIGS. 2 and 5, the software recognizes the windows and crops a section thereof for analysis and counting. In one preferred embodiment, such as that illustrated in FIG. 2, the target zones or windows have the shape of 1×2 mm rectangles with a semicircular section on each end thereof. In this embodiment, the software crops a standard rectangle of 1×2 mm area inside a respective window. In an aspect of this embodiment, the reader may take several consecutive sample values to compare the number of cells in several different windows.

In embodiments of the invention using a transmissive disc without windows, as shown in FIGS. 5, 6, 8, and 9, step 246 may be performed in one of two different manners. The position of the standard rectangle is chosen either by positioning its center relative to a point with fixed coordinates, or by finding reference mark 202 (see for example FIGS. 24L, 25, and 26), which is a spot of dark dye. In the case where a reference mark 202 is employed, a dye with a desired contrast is deposited in a specific position 141 (FIG. 20E for example) on the disc with respect to two clusters of cells. The optical disc reader is then directed to skip to the center of one of the clusters of cells and the standard rectangle is then centered around the selected cluster.

As for the user options mentioned above in regard to step 246, the user may specify a desired sample area shape for cell counting, such as a rectangular area, by direct interaction with mouse selection or otherwise. In the present embodiment of the software, this involves using the mouse to click and drag a rectangle over the desired portion of the optical bio-disc-derived image that is displayed on a monitor 114. Regardless of the evaluation area selection method, a respective rectangular area is evaluated for counting in the next step 248.

The third principal step in FIG. 58 is step 248, which is directed to background illumination uniformization. This process corrects possible background uniformity fluctuations caused in some hardware configurations. Background illumination uniformization offsets the intensity level of each sample point such that the overall background, or the portion of the image that is not cells, approaches a plane with an arbitrary background value V_{background}. While V_{background} may be decided in many ways, such as taking the average value over the standard rectangular sample area, in the present embodiment, the value is set to 2000. The value V at each point P of the selected rectangular sample area is replaced with the number (V_{background}+ (V−average value over the neighborhood of P)) and truncated, if necessary, to fit the actual possible range of values, which is 0 to 4095 in a preferred embodiment of the present invention. The dimensions of the neighborhood are chosen to be sufficiently larger than the size of a cell and sufficiently smaller than the size of the standard rectangle.

The next step in the flow chart of FIG. 58 is a normalization step 250. In conducting normalization step 250, a linear transform is performed with the data in the standard rectangular sample area so that the average becomes 2000 with a standard deviation of 600. If necessary, the values are truncated to fit the range 0 to 4096. This step 250, as well as the background illumination uniformization step 248, makes the software less sensitive to hardware modifications and tuning. By way of example and not limitation, the signal gain in the detection circuitry, such as top detector 158 (FIG. 55), may change without significantly affecting the resultant cell counts.

As shown in FIG. 58, a filtering step 252 is next performed. For each point P in the standard rectangle, the number of points in the neighborhood of P, with dimensions smaller than indicated in step 248, with values sufficiently distinct from V_{background} is calculated. The points calculated should approximate the size of a cell in the image. If this number is large enough, the value at P remains as it was; otherwise it is assigned to V_{background}. This filtering operation is performed to remove noise, and in the optimal case only cells remain in the image while the background is uniformly equal V_{background}.

An optional step 254 directed to removing bad components may be performed as indicated in FIG. 58. Defects such as scratches, bubbles, dirt, and other similar irregularities may pass through filtering step 252. These defects may cause cell counting errors either directly or by affecting the overall distribution in the images histogram. Typically, these defects are sufficiently larger in size than cells and can be removed in step 254 as follows. First a binary image with the same dimensions as the selected region is formed. A in the binary image is defined as white, if the value at the corresponding point of the original image is equal to V_{background}, and black otherwise. Next, connected components of black points are extracted. Then subsequent erosion and expansion are applied to regularize the view of components. And finally, components that are larger than a defined threshold are removed. In one embodiment of this optional step, the component is removed from the original image by assigning the corresponding sample points in the original image with the value V_{background}. The threshold that determines which components constitute countable objects and which are to be removed is a user-defined value. This threshold may also vary depending on the investigational feature being counted i.e. white blood cells, red blood cells, or other biological matter. After optional step 254, steps 248, 250, and 252 are preferably repeated.

The next principal processing step shown in FIG. 58 is step 256, which is directed to counting cells by bright centers. The counting step 256 consists of several substeps. The first of these substeps includes performing a convolution. In this convolution substep, an auxiliary array referred to as a convolved picture is formed. The value of the convolved picture at point P is the result of integration of a picture after filtering in the circular neighborhood of P. More precisely, for one specific embodiment, the function that is integrated, is the function that equals v−2000 when v is greater than 2000 and 0 when v is less than or equal to 2000. The next substep performed in counting step 256 is finding the local maxima of the convolved picture in the neighborhood of a radius about the size of a cell. Next, duplicate local maxima with the same value in a closed neighborhood of each other are avoided. In the last substep in counting step 256, the remaining local maxima are declared to mark cells.

In some hardware configurations, some cells may appear without bright centers. In these instances, only a dark rim is visible and the following two optional steps 258 and 260 are useful.

Step 258 is directed to removing found cells from the picture. In step 258, the circular region around the center of each found cell is filled with the value 2000 so that the cells with both bright centers and dark rims would not be found twice.

Step 260 is directed to counting additional cells by dark rims. Two transforms are made with the image after step 258. In the first substep of this routine, substep (a), the value v at each point is replaced with (2000−v) and if the result is negative it is replaced with zero. In substep (b), the resulting picture is then convolved with a ring of inner radius R1 and outer radius R2. R1 and R2 are, respectively, the minimal and the maximal expected radius of a cell, the ring being shifted, subsequently, in substep (d) to the left, right, up and down. In substep (c), the results of four shifts are summed. After this transform, the image of a dark rim cell looks like a four petal flower. Finally in substep (d), maxima of the function obtained in substep (c) are found in a manner to that employed in counting step 256. They are declared to mark cells omitted in step 256.

After counting step 256, or after counting step 260 when optionally employed, the last principal step illustrated in FIG. 58 is a results output step 262. The number of cells found in the standard rectangle is displayed on the monitor 114 shown in FIGS. 1 and 5, and each cell identified is marked with a red cross on the displayed optical bio-disc-derived image.

With reference to FIG. 59, there is shown an illustration of red blood cells or erythrocytes and white blood cells or leukocytes. Leukocytes or white blood cells fall into two major groups. The first group is the granular leukocytes. They develop from red bone marrow, have conspicuous granules in the cytoplasm, and possess lobed nuclei. The three kinds of granular leukocytes (also know as polymorphs, polymorphonuclear leukocytes, or PMNS) are neutrophils (10 to 12 um in diameter), eosinophils (10 to 12 um in diameter), and Basophils (8 to 10 um in Diameter). The nuclei of neutrophils have two to six lobes, connected by very thin strands. As the cell ages the extent of lobulation increases. Fine, evenly distributed pale lilac colored granules are found in the cytoplasm when a commonly used stain is applied to the cells. Eosinophils contain nuclei that are also bilobed, with the lobes connected by a thin strand or thick isthmus. The cytoplasm is packed with large uniform-sized granules that do not cover or obscure the nucleus. The granules stain red-orange with a commonly used stain. The nuclei of basophils are also bibbed or irregular in shape, often in the form of a letter S. The cytoplasmic granules are round, variable in size, stain blue-black, and commonly obscure the nucleus.

The second principal group of leukocytes are agranular leukocytes. They develop from lymphoid and myeloid tissue (red bone marrow), and no cytoplasmic granules can be seen under a light microscope, owing to their small size and poor staining qualities. The two kinds of agranular leukocytes are lymphocytes (7 to 15 um in diameter) and monocytes (14 to 19 um in diameter). The nuclei of lymphocytes are round, or slightly indented. The cytoplasm forms a ring around the nucleus. The nuclei of monocytes are usually indented or kidney shaped as shown in FIGS. 59, 60, and 61.

According to the present invention, certain pre-determined cellular compartments may be stained with a dye having a pre-determined absorbance range. The cellular compartments may include the nucleus, nucleolus, nuclear envelope, golgi apparatus, endoplasmic reticulum, mitochondria, vacuoles, peroxisomes, microtubules, centrioles, ribosomes, cell membrane, and cell wall. The pre-determined wavelength may include UV, visible, and IR absorbance ranges. These dyes may include vital, infrared, near-infrared, and fluorescent dyes may be employed to enhance the contrast between different cellular compartments and allow differentiation and quantitation of various cells is a sample. For example, various white blood cells types may be identified and quantified based on the morphology and number of their nuclei, when the nucleus is stained. The incident or interrogation beam used to detect the cells and the stained compartments preferably has a wavelength within 10 nm of the maximum absorbance wavelength of the dye used. Details relating to cell detecting, counting, and image recognition is disclosed in co-pending, commonly assigned U.S. provisional application serial Nos. 60/356,982, filed Feb., 13, 2002; No. 60/372,007, filed Apr. 11, 2002; No. 60/___,___, filed Sep. 4, 2002; all entitled “Bio-Disc and Bio-Drive Analyser System Including Methods Relating Thereto”; U.S. patent application No. 10/008,156, entitled “Disc Drive System and Methods for Use with Bio-discs”, filed Nov. 9, 2001; and U.S. patent application No.10/043,688, entitled “Optical Disc Analysis System Including Related Methods For Biological and Medical Imaging”, filed Jan. 10, 2002. All of these applications are herein incorporated by reference in their entireties.

In accordance with one of the preferred embodiments of the present invention, infrared absorbing dyes including LI-COR IRDyes like NN382, IRDye38, IRDye40, IRDye41, IRDye78, IRDye80, IRDye700, and IRDye800 (LI-COR Biosciences, Inc., Lincoln, NE), TO-PRO-5-iodide, IR-780 iodide, Laser Pro IR Dyes (Molecular Probes, Eugene, Oreg.), dd-007, Zynostain (Zynocyte, Univ. of Glasgow, U.K.), idocyanine green, copper phthalocyanine, 3,3′-diethylthiatricarbocyanine iodide (DTTCI), 3,3′-diethyloxatricarbocyanine iodide (DOTCI), 3,3′-diethylthiadicarbocyanine iodide (DTDCI), and 3,3′-diethyloxadicarbocyanine iodide (DODCI) dyes are used to label the cell nucleus so as to improve the image of the nucleus of the cells in the sample. This staining process facilitates visualization of additional information and details of normal and abnormal cells in the samples. Other dyes may be used to stain various parts of the cell. Examples of other dyes that can be used in the present invention are described and listed in “The Sigma-Aldrich Handbook of Stains, Dyes, and Indicators” by Floyd J. Green, Aldrich Chemical Inc., Milwaukee, Wis.; “Topics in Applied Chemistry: Infrared Absorbing Dyes, Masaru Matsuoka (Ed.), Plenum Press, New York, N.Y.; and in Patonay, G, and Antoine, M. D., “Near-infrared Fluorogenic Labels: New Approach to an Old Problem”, Analytical Chemistry, Vol. 63, No. 6, Mar. 15, 1991. All of which are incorporated in their entireties herein.

In yet another preferred embodiment of the present invention the staining process involving a vital nuclear stain including Zynostain to increase contrast of nucleus to the surrounding cytoplasm and improve the image quality and aid in identification of the various white blood cell types.

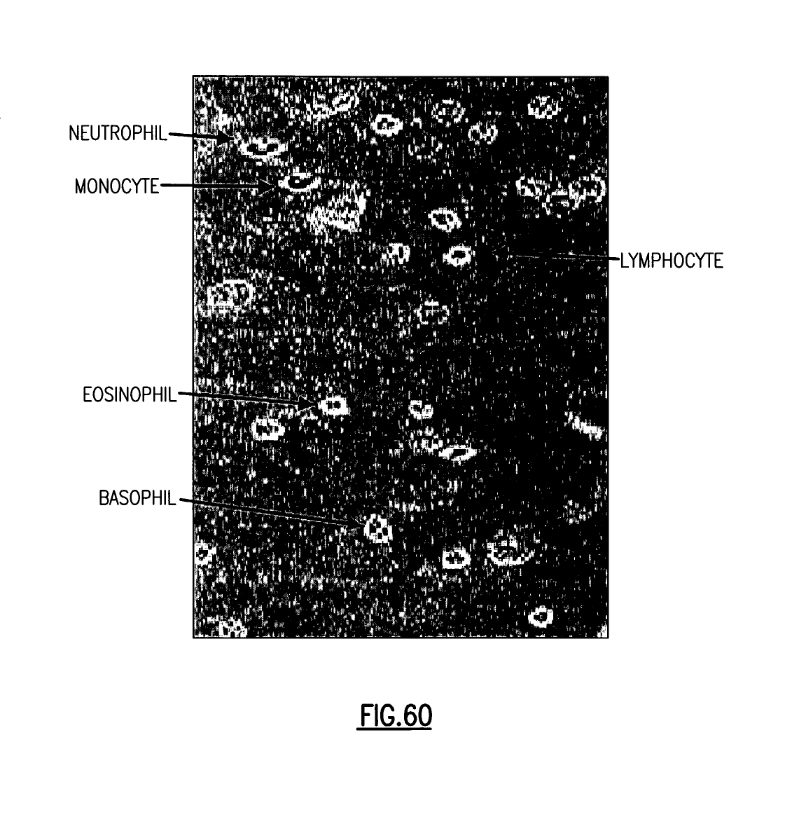

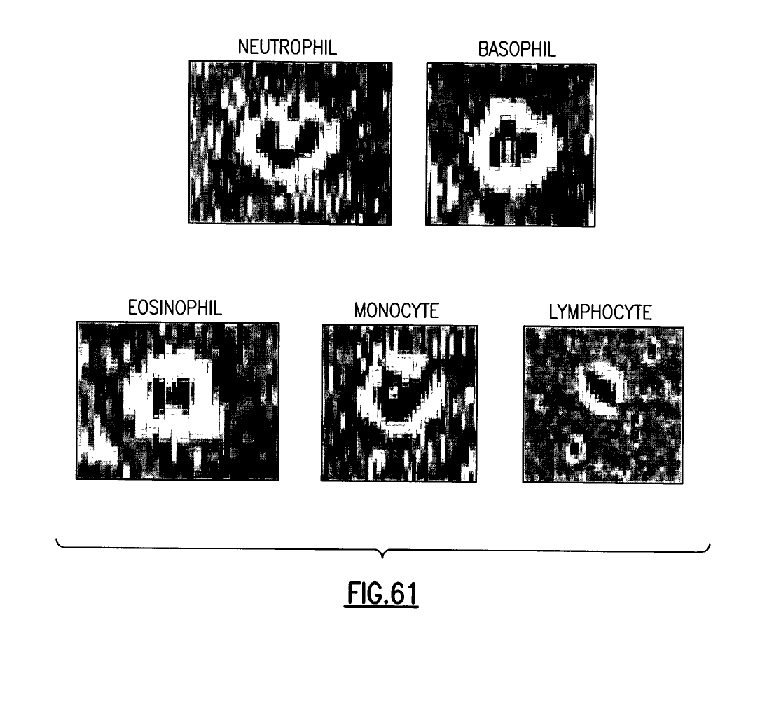

Referring next to FIG. 60, there is shown an image of white blood cells stained with dd-007 infrared dye collected using the optical bio-disc system of the present invention. As shown, the cell nuclei are stained and are opaque to the incident beam from the optical disc reader. The opacity of the nuclei is due to the absorption of the incident beam by the dd-007 dye. As illustrated, the various cell types in the sample may be readily identified based on the morphology of their nuclei. A magnified image of the cells identified in FIG. 60 are shown in FIG. 61.

Experimental Details

While this invention has been described in detail with reference to the drawing figures, certain examples and further illustrations of the invention are presented below.

### EXAMPLE 1

FIG. 17A illustrates a pictorial flow chart showing the preparation of a sample, use of a bio-disc, and the provision of results which are shown in greater detail in FIGS. 52 and 53. The details of the following example such as the individual time duration of process steps, rotation rates, and other details are more particular than those described above with reference to FIGS. 17A, 52, and 53. The basic steps of the present example are, nonetheless, similar to those described above.

A. Disc Manufacturing Including Substrate Preparation and Chemistry Deposition

In this example, a reflective disc or transmissive disc substrate 120 (FIGS. 2 and 5, respectively) is cleaned using an air gun to remove any dust particles. The disc is rinsed twice with iso-propanol, using a spin coater. A 2% polystyrene is spin coated on the disc to give a very thick coating throughout.

The chemistry is then deposited. One embodiment includes a three step deposition protocol that incubates: streptavidin, incubated for 30 minutes; biotinylated first antibody incubated for 60 minutes; and a second capture antibody incubated for 30 minutes. The first antibody can be raised in a first species (e.g., sheep) against a type of immunoglobulin (e.g., IgG, IgE, IgM) of a second species (e.g., mouse). The second capture antibody is raised in the second species against a specific cell surface antigen (e.g., CD4, CD8). The steps are done at room temperature in a humidity chamber using washing and drying steps between depositions.

A 1 μl ratio of 1 mg/ml streptavidin in phosphate buffered saline is layered over each window and incubated for 30 minutes. Excess streptavidin is rinsed off using distilled water and the disc is dried. Equal volumes of biotinylated anti-mouse IgG (125 μg/ml in PBS) and activated dextran aldehyde (200 μg/ml) are combined. Dextran aldehyde (DCHO)-biotinylated anti-mouse IgG is layered over streptavidin in each capture window and incubated for 60 minutes or overnight in refrigerator. Excess reagent is rinsed and the disc is spun dry.

As shown in FIG. 47, there can be a number of radially oriented viewing windows with different tests, such as CD4 (window 2), CD8 (window 3), CD3 (window 4), and CD45 (window 5), and negative control (window 6), using mouse IgG antibodies against these human cell surface antigens. This prepared substrate is incubated for 30 minutes or overnight in the refrigerator.

The pattern of chemistry deposition is provided below in Table 3.

TABLE 3

| Window | 1-2 | 3-4 | 5-6 | 7-8 |
|---|---|---|---|---|
| 1^{st} Layer | Streptavidin | Streptavidin | Streptavidin | Streptavidin |
| Secondary | B-anti Mouse | B-anti Mouse | B-anti | B-anti |
| Antibodies | IgG+ DCHO | IgG+ DCHO | Mouse | Mouse |
| | | | IgG+ DCHO | IgG+ DCHO |
| Primary | Mouse Anti- | Mouse Anti- | Mouse Anti- | Mouse Anti- |
| Antibodies | human CD4 | human CD8 | human CD3 | human CD45 |

B. Disc Assembly

The disc is assembled using an adhesive layer that may, for example, be 25, 50, or 100 microns thick (channel layer 118 in FIGS. 2 and 5), with a stamped out portion, such as a U-shape or “e-rad” channel, to create a fluidic channel, and a clear cap 116 (FIG. 5, for use with a transmissive disc with a top detector) or a cap 116 with a reflective layer 142 located over the capture zones (FIG. 2, for use with a reflective disc with a bottom detector).

In one embodiment, the disc is a forward Wobble Set FDL21:13707 or FDL21:1270 CD-R disc coated with 300 nm of gold as the encoded information layer. On a reflective disc, viewing windows of size 2×16 mm oval are etched out of the reflective layer by known lithography techniques. In some designs of transmissive disc, no separate viewing windows are etched, and the entire disc is available for use. In this particular example, the channel layer is formed from Fraylock adhesive DBL 201 Rev C 3M94661. The cover is a clear disc with 48 sample inlets each with a diameter of 0.040 inches located equidistantly at radius 26 mm. The data disc is scanned and read with the software at speed 4× and sample rate 2.67 MHz using CD4/CD8 counting software.

C. Disc Leak Check

Because blood is being analyzed, the disc can be leak checked first to make sure none of the chambers leak during spinning of the disc with the sample in situ. Each channel is filled with a blocking agent, such as StabilGuard and PBS-Tween. The block is for at least 1 hour. The disc is spun at 5000 rpm for 5 minutes to leak proof and check disc stability. After checking for leaks, the disc is placed in a vacuum chamber for 24 hours. After vacuuming for 24 hours, discs are placed in a vacuum pouch and stored in refrigerator until use.

D. Sample Collection, Preparation, and Application to Disc

The following section is directed to sample processing steps which are generally shown in FIG. 17A. Mononuclear cells (MNC) are purified by a density gradient centrifugation method, e.g., using a Becton Dickinson CPT Vacutainer. Blood (4-8 ml) is collected directly into a 4 or 8 ml EDTA containing CPT Vacutainer. The tubes are centrifuged at 1500 to 1800×g in a biohazard centrifuge with horizontal rotor and swing out buckets for 25 minutes at room temperature. The blood is preferably used within two hours of collection. After centrifugation, plasma overlying the mononuclear cell fraction is removed, leaving about 2 mm of plasma above an MNC layer. MNC are collected and washed with PBS. Cells are pelleted by centrifuge at 300×g for 10 minutes at room temperature. The supernatant is removed and the pellet containing the MNC is resuspended in PBS by tapping the tube gently. One more washes are done at 300×g for 10 minutes each at room temperature to remove platelets. The final pellet is resuspended to a cell count of 10,000 cells/μl. An 18μl volume of the MNC is introduced to one or more the analysis chamber or channel, incubated for 15 minutes at room temperature with the disc stationary. The channels are sealed. The disc is then spun at 3000 rpm for 3 to 4 minutes using a disc drive. The disc is preferably scanned and read with the software at speed 4× and sample rate 2.67 MHz.

If a blood sample cannot be processed immediately, mononuclear cells after the first centrifugation can be resuspended in plasma by gently inverting the CPT tube several times and stored for up to 24 hours at room temperature. Within 24 hours, the cells in the plasma can be collected and washed as described above.

E. CD4/CD8 Assay Format

The assay in this example is a generic homogeneous solid phase cell capture assay for the rapid determination of absolute number of CD4+ and CD8+ T-lymphocyte populations and ratio of CD4+/CD8+ lymphocytes in blood samples. The test, which is run within a small chamber incorporated into a CD-ROM, determines the number of CD4+, CD8+, CD2+, CD3+ and CD45+ cells captured by the specific antibodies on the capture zones in 7 μl of mononuclear cells (MNC) isolated from whole blood. The test is based upon the principle of localized cell capture on specific locations on the disc. Several specific cell capture zones are created on the disc by localized application of capture chemistries based upon monoclonal or polyclonal antibodies to particular blood cell surface antigens. Upon flooding the chamber with the MNC blood (30,000 cells/μl), cells expressing antigens CD4, CD8, CD2, CD3 and CD45 are captured in the capture zones on the disc. Also incorporated within the bar code are defined negative control areas.

F. On-Disc Analysis

MNC cells, prepared in step D above (18 μl in PBS), are injected into the disc chamber, and inlet and outlet ports of the chamber are sealed. The disc is incubated for 15 minutes at room temperature, and then scanned using a 780 nm laser in an optical drive with a top detector to image the capture field as described above.

Software is encoded on the disc to instruct the drive to automatically perform the following acts: (a) centrifuge the disc to spin off excess unbound cells in one or more stages, (b) image specific capture windows, and (c) process data including counting the specifically-captured cells in each capture zone and deriving the ratio of CD4/CD8 (or which ever ratio is programmed to be determined).

During the processing step, the software reads across each capture zone image and marks cells as it encounters them. For example, following estimation of number of CD4+ and CD8+ cells, the software calculates the ratio of CD4+/CD8+ cells and displays both the absolute numbers of cells in CD4+, CD8+, CD3+ and CD45+ capture zones per microliter of whole blood and also the CD4+/CD8+ ratio. The entire process takes about 12 minutes from inserting the disc into the optical drive to obtaining the numbers and ratios.

G. Reagents Used

Streptavidin (Sigma, cat. # S-4762): Add de-ionized Water to make a 5 mg/ml solution, aliquot and store at −30° C. To use, add Tris buffer for a final concentration of 1 mg/ml.

Positive control: CD45 (Sigma, Lot # 038H4892, cat # C7556). Store at 2-8° C.

Secondary capture antibody: Biotinylated anti-mouse IgG (raised in sheep, Vector laboratories, lot # L0602, Catalog # BA-9200) Stock solution 1.5 mg/ml made in distilled water. Working b-IgG solution 125 μg/ml in 0.1M PBS. Store at 2-8° C. May be kept at −30° C. for long term storage.

Aldehyde activated Dextran (Pierce, lot # 97111761, cat # 1856167). Stock solution stock solution 5 mg/ml in PBS, store at 2-8° C.

Primary capture antibody: CD4 (DAKO, cat # M0716), CD8 (DAKO, cat # M0707), CD2 (DAKO, cat # M720), CD45 (DAKO, cat # M0701), CD14 (DAKO, cat # M825), and CD3 (DAKO, cat # M7193). Store at 2-8° C.

Negative control: Mouse IgG1 (DAKO, cat # X0931). Store at 2-8° C.

Phosphate Buffered Saline (PBS), pH 7.4 (Life Technologies/GIBCO BRL, cat. # 10010-023) or equivalent. Store at room temperature Isopropyl alcohol, 90-100%

H. RBC Lysing Protocol

Ammonium Chloride Lysing Buffer

A 1× stock of ammonium chloride lysing buffer should be stored at 2 to 8° C. Comprised of 0.155M NH₄Cl, 10 mM KHCO₃, and 0.1 mM disodium EDTA; pH7.3 to 7.4. Store at 2-8° C. Bring to room temperature prior to use.

Procedure

1. For every 100 μl of blood add 2 ml of lysing buffer. (It is preferable to do this procedure in a biohazard hood.)

2. Vortex and incubate for 15 minutes at room temperature.

3. Centrifuge the blood at 500×g for 5 minutes at room temperature, using the centrifuge in the biohazard hood.

4. Remove supernatant and wash cells with 2% FCS or FBS in PBS. Centrifuge cells.

5. Calculate the total amount of WBCs and make the final concentration of WBCs 10,000 cells/μl for sample injection.

### EXAMPLE 2

### Mononuclear Cells Separation Procedure

Use Becton and Dickinson Vacutainer CPT (BD catalog # 362760 for 4 ml, # 362761 for 8 ml) cell preparation tubes with sodium citrate. Do procedure in biohazard hood following all biohazard precautions. Steps:

1. Collect blood directly into a 4 or 8 ml EDTA containing CPT Vacutainer. If the blood sample is already in an anticoagulant, pour off the EDTA in the Vacutainer first and then pour 6-8 mls of blood sample into the CPT tube.

2. Centrifuge the tube at 1500 to 1800×g in a biohazard centrifuge with horizontal rotor and swing out buckets for 25 minutes at room temperature. For best results, the blood should be centrifuged within two hours of collection. However, blood older then 2 hours may be centrifuged with a decrease in MNC number and increase in RBC contamination.

3. After centrifugation, remove the plasma leaving about 2 mm of plasma above the MNC layer. Collect and transfer the whitish mononuclear layer into a 15 ml conical centrifuge tube.

4. Add 10-15 mls with PBS to MNC layer, gently mix the cells by inverting the centrifuge tube several times.

5. Wash cells by centrifuge at 200×g for 10 minutes at room temperature in biohazard centrifuge.

6. Remove supernatant. Resuspend cells by tapping the tube gently.

7. Wash one more time in 10 ml of PBS. Centrifuge at 200-300×g for 10 minutes at room temperature to remove platelets.

8. Remove supernatant and resuspend pellet in 50 ul PBS.

9. Estimate cell counts in the sample. Run CBC or dilute 2 ul of cells to 18 ul of trypan blue, gently mix and count cells with a hemocytometer. Make up the sample to a final cell count of 10,000 cells/ul for analysis.

10. If the cells cannot be processed immediately, resuspend mononuclear cells after the first centrifugation (step 2 above) in the separated plasma by gently inverting the CPT tube several times and store for up to 24 hours at room temperature. Within 24 hours, collect the cells in the plasma and continue with the washes as described above.

Total cell counts per ul =number of cells in 25 small squares×(times) 100.

### EXAMPLE 3

### Isolation of MNCs from Whole Blood Using Histopaque-1077

1 ml of Histopaque-1077 was placed in a 15 ml centrifuge tube and 1 ml of whole blood is gently layered over that. Then centrifuged at 400×g for 30 min at room temperature. The mixture was aspirated carefully with a pasture pipette and the opaque interface transferred to centrifuged tube. Then 10 ml of PBS was added to the centrifuge tube. The solution was then centrifuged at 250×g for 10 min. The supernatant was decanted and the cell pellet was resuspended in 10.0 ml PBS and spun at 250×g for 10 min. The cells were then washed one more time by resuspending the pellet in 10 ml PBS and spinning at 250×g. The final cell pellet was resuspended in 0.5 ml PBS.

### EXAMPLE 4

### Disc Preparation and Chemistry Deposition (With Streptavidin)

A. Disc Manufacturing Including Substrate Preparation and Chemistry Deposition

In this example, a transmissive disc substrate was cleaned with an air gun to remove dust. The disc was then mounted in the spin coater and rinsed twice with a steady stream of iso-propanol. Next, a polystyrene solution with 2% polystyrene dissolved in 310 mls of toluene and 65 mls of iso-propanol was evenly coated onto the disc.

For the streptavidin deposition, streptavidin stock solution was diluted to 1mg/ml in PBS. Using manual pin deposition, approximately 1 ul of the streptavidin was deposited in each capture zone on the disc. The disc was incubated in a humidity chamber for 30 minutes. Then excess unbound streptavidin was rinsed off the capture zones with D. I. water and the disc was spun dried.

For the secondary antibody deposition, a fresh solution of activated dextran aldehyde (200 ug/ml in PBS) was combined with an equal volume of the Vector IgG (125 ug/ml in PBS). Using manual pin deposition, approximately 1 ul of the IgG+DCHO complex was deposited (layered on top of the streptavidin layer) in each capture zone on the disc. The disc was incubated in a humidity chamber for 60 minutes. Excess antibody was rinsed off with D. I. water and the disc was spun dry.

For the primary antibody, DAKO CD4 was diluted to 50 ug/ml in PBS, DAKO CD8 was diluted to 25 ug/ml in PBS, and DAKO CD45 was diluted to 145 ug/ml in PBS. Using the manual pin applicator, deposited approximately 1 ul of each primary antibody on top of the bound secondary antibodies. The disc was then incubated in a humidity chamber for 30 minutes. The excess unbound antibody was removed by washing the capture zones with PBS and the disc was spun dried.

B. Disc Assembly

The cover disc used was a clear disc with a Fraylock adhesive channel layer attached thereto. Stamped into the adhesive were 4 U-shaped channels that created the fluidic circuits. The cover was placed onto the transmissive disc substrate so that the fluid channels were over the capture zones. Next, to secure the discs together, they were passed through a disc press 8 times.

C. Disc Leak Check, Blocking

Each fluid channel was filled with StableGuard and incubated for 1 hour. During the incubation, the disc was spun in the spin coater for 5 minutes at 5000 rpm. After the spin, the disc channels were checked for leaks. Next, the StableGuard was aspirated out of the channels, and the disc was placed under vacuum in a vacuum chamber overnight. The next morning, the disc was placed in a vacuum pouch and stored at 4° C.

### EXAMPLE 5

### Preparation of Blood Smear

5 μl blood was taken from the Buffy coat containing RBC, a large number of WBC and platelets. The cells were placed on one end of the cover slip in the middle, placed one end of the other cover slip over the blood drop for spreading the blood along the edge and then slide over evenly, the cells were fixed in 100% ethanol and air dried.

### EXAMPLE 6

### Staining Cells with Zynostain

A. 25 μl blood cells was taken from buffy coat and stained with 25 μl of vital dye Zynostain by using 1% ethanol, 1% TritonX 100 (50 μl /5 ml Po₄ buffer) and using incubation temperature 37° C., sample were taken out at 10, 20 and 30 mins. The cells were then smeared on a glass cover slip. The cover slip was attached to an optical bio-disc and images of then cells were taken using an optical bio-disc system.

B. Blood cell was taken from buffy coat and mixed with Zynostain in 1:3 and 1:6 of cell to stain ratio and incubated for 5, 10, and 30 mins. Then made a smear on a glass cover slip. The cover slip was attached to an optical bio-disc and images of then cells were taken using an optical bio-disc system. We found 1:6 cell to stain ratio and 30 min. incubation shows good cell staining.

C. 5 μl of whole blood was taken and mixed with 20 μl of leukemia culture cells and 120 μl of Zynostain and incubated for 30 mins. Then made a smear on a glass cover slip. The cover slip was attached to an optical bio-disc and images of then cells were taken using an optical bio-disc system.

### EXAMPLE 7

### Staining Lymphocyte Culture Cells (KG1a) with To-Pro-5-Iodide Fluorescent Dye

The cell culture was centrifuged for 5 min. at 800 rpm. The supernatant was removed and the pellet was resuspended in PBS pH 7.4. Then 10 μl of TP-Pro-5-iodide and 100 μl of cell suspension was mixed. Cells were then observed under a fluorescent microscope after 5, 10, 30 mins. of incubation with To-Pro-5-iodide.

### EXAMPLE 8

### Staining KG1a Cells with LI- COR IRDye38

KG1a culture cells were washed twice (2×10 mins spins at 800 rpm) in PBS pH 7.4. The washed cells were incubated in 4% IRDye38 in DI water for 30 mins. then smeared on a glass cover slip. The cover slip was attached to an optical bio-disc and images of then cells were taken using an optical bio-disc system.

### EXAMPLE 9

### Staining KG1a Cells with IRDye38 and dd-007

Isolated KG1a cells, spun culture cells at 800 rpm for 5 min., decanted the supernatant and washed the cells 2×10 min. in PBS pH 7.4. The washed cells were divided into two aliquots and mixed with either 4% IRDye38 in DI water or dd-007 in TritonX-PBS 7.4 each for 30 mins. The cells were then smeared on a glass cover slip. The cover slip was attached to an optical bio-disc and images of then cells were taken using an optical bio-disc system. The result from this experiment using the dd-007 dye is shown in FIGS. 60 and 61.

### Concluding Statements

Aspects of the present invention relating to the apparatus, methods, and processes disclosed herein are also presented in U.S. Provisional Application Serial No. 60/323,682 entitled “Methods for Reducing Non-Specific Binding of Cells on Optical Bio-Discs Utilizing Blocking Agents” filed Sep. 20, 2001; U.S. Provisional Application Serial No. 60/324,336 entitled “Methods for Reducing Bubbles in Fluidic Chambers Using Polyvinyl Alcohol and Related Techniques for Achieving Same in Optical Bio-Discs” filed Sep. 24, 2001; U.S. Provisional Application Serial No. 60/326,800 entitled “Sealing Methods for Containment of Hazardous Biological Materials within Optical Analysis Disc Assemblies” filed Oct. 3, 2001; U.S. Provisional Application Serial No. 60/328,246 entitled “Methods for Calculating Qualitative and Quantitative Ratios of Helper/Inducer-Suppressor/Cytotoxic T-Lymphocytes Using Optical Bio-Disc Platform” filed Oct. 10, 2001; U.S. Provisional Application Serial No. 60/386,072 entitled “Quantitative and Qualitative Methods for Characterizing Cancerous Blood Cells Including Leukemic Blood Samples Using Optical Bio-Disc Platform” filed Oct. 19, 2001; U.S. Provisional Application Serial No. 60/386,073 entitled “Methods for Quantitative and Qualitative Characterization of Cancerous Blood Cells Including Lymphoma Blood Samples Using Optical Bio-Disc Platform” filed Oct. 19, 2001; U.S. Provisional Application Serial No. 60/386,071 entitled “Methods for Specific Cell Capture by Off-Site Incubation of Primary Antibodies with Sample and Subsequent Capture by Surface-Bound Secondary Antibodies and Optical Bio-Disc Including Same” filed Oct. 26, 2001; U.S. Provisional Application Serial No. 60/344,977 entitled “Quantitative and Qualitative Methods for Cell Isolation and Typing Including Immunophenotyping” filed Nov. 7, 2001; and U.S. Provisional Application Serial No. 60/349,975 entitled “Methods for Reducing Non-Specific Binding of Cells on Optical Bio-Discs Utilizing Charged Matter Including Heparin, Plasma, or Poly-Lysine” filed Nov. 9, 2001, all of which are herein incorporated by reference.

All patents, provisional applications, patent applications, and other publications mentioned in this specification are incorporated herein in their entireties by reference.

While this invention has been described in detail with reference to a certain preferred embodiments, it should be appreciated that the present invention is not limited to those precise embodiments. Rather, in view of the present optical bio-disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein. Such equivalents are also intended to be encompassed by the following claims.

What is claimed is:

1. A method of conducting an assay employing an optical disc and disc drive, said method comprising the steps of:
providing a sample of cells on a disc surface in a chamber in a disc, the chamber including at least one capture zone with a capture agent;
loading the disc into an optical reader;
rotating the optical disc;
directing an incident beam of electromagnetic radiation to the capture zone;
detecting a beam of electromagnetic radiation formed after interacting with the disc at the capture zone;
converting the detected beam into an output signal; and
analyzing the output signal to extract therefrom information relating to the number and type of cells captured at the capture zone.

2. An optical disc and drive system for receiving a sample, the system comprising:
a disc including a substrate, a cap parallel to the substrate, and a chamber defined between said cap and substrate including capture zones;
a capture layer over the substrate at the capture zones, a first capture zone having first cell capture agents and a second capture zone having a second cell capture agents;
a light source for directing light toward the disc at the capture zones;
a detector for detecting light reflected from or transmitted through the disc at the capture zones and providing a signal; and
a processor for using the signal to distinguish the nuclei of captured cells and count items in the sample bound to the capture molecules.

3. A method of performing a white blood cell count employing an optical disc and disc drive, said method comprising the steps of:
providing a blood sample in a first tube, the first tube containing a separation gradient;
rotating the first tube at a time and speed sufficient to separate the blood sample into layers;
isolating a white blood cell layer from the separated blood sample;
resuspending the white blood cell layer thereby forming a white blood cell suspension;
providing a sample of the white blood cell suspension on an optical disc surface, the surface including at least one capture zone with at least one capture agent;
loading the optical disc into an optical reader;
rotating the optical disc;
directing an incident beam of electromagnetic radiation to a capture zone;
detecting a beam of electromagnetic radiation formed after interacting with the disc at the capture zone;
converting the detected beam into an output signal; and
analyzing the output signal to extract therefrom information relating to the morphology of the nuclei of the cells captured at the capture zone.

4. The method according to claim 3 further comprising the steps of:
directing the sample of white blood cells into proximity with the capture agents;
incubating the cells in the presence of the capture agents;
allowing the cells to specifically bind to the capture agents;
staining nucleus of the cells with a stain that absorbs at or near the wavelength of the beam of electromagnetic radiation.

5. The method according to claim 4 further comprising the step of analyzing the number of cells captured of a specific type to thereby determine a concentration of said specific cell type in the sample.

6. The method according to claim 5 wherein the said step of analyzing the morphology of the nuclei of captured cells comprises detecting changes in the level of light reflected from or transmitted through the disc.

7. The method according to claim 6 further including the step of counting the number of cells of a specific cell type using image recognition to distinguish cells types by nucleus morphology.

8. The method according to claim 7 wherein the image recognition comprises distinguishing one type of white blood cell nucleus from another.

9. The method according to claim 8 wherein the image recognition comprises using dyes for cell staining.

10. The method according to claim 9 wherein said dyes are selected from the group comprising vital dyes, fluorescent dyes, infrared and near-infrared dyes.

11. The method according to claim 10 wherein said vital dyes are selected from the group comprising Leishman's, acridine orange, and Zynostain.

12. The method according to claim 3 wherein said step of rotating the optical disc includes rotating for a sufficient period of time at a sufficient speed so that the cells have an opportunity to bind with said at least one capture agent.

13. The method according to claim 12 wherein said step of rotating the optical disc further includes rotating for a sufficient period of time at a sufficient speed so that unbound cells are moved away from the capture zones.

14. The method according to claim 13 wherein said step of rotating the optical disc is done at a single speed.

15. The method according to claim 3 further comprising the step of counting the captured cells in each of the capture zones and providing an output including cell counts.

16. The method according to claim 15 wherein the output includes counts for CD4 cells and CD8 cells, and a ratio of CD4 to CD8 cells.

17. An optical bio-disc employed for performing cluster designation counts in accordance with the methods recited in any one of claims 3 to 16.

18. A method of making an optical assay disc for performing a differential white blood cell count using nucleus morphology to distinguish cell types, said method comprising the steps of:
providing a substrate;
coating said substrate with an active layer;
providing a cross-linker on said active layer to thereby create one or more capture zones;
allowing said cross-linker to bind to said active layer;
removing excess cross-linker from the capture zones; and
attaching a cap portion to said active layer to form a channel adapted to receive a suspension of cells and a dye solution for staining the nuclei of captured cells.

19. A method of analyzing white blood cells in a test sample, said method comprising the steps of:
loading a plurality of white blood cells into an optical bio-disc;
capturing said white blood cells in designated target zones by use of capture agents having specificity for particular cell surface markers;
staining the nuclei of the captured white blood cells with a dye;
directing an incident beam of light toward said captured white blood cells;
allowing said incident beam of light to interact with the stained nuclei of the captured white blood cells to thereby form a return beam of light carrying information regarding the morphology of the nuclei;
detecting said return beam of light;
converting said detected return beam into an output signal; and
analyzing said output signal to extract therefrom information relating to the morphology of the nuclei of the cells captured at the capture zone.

20. The method according to claim 19 wherein said dye is selected from the group of dyes comprising vital, vital nuclear, nuclear, DNA, chromosomal, fluorescent, infrared, near-infrared, UV, and visible dyes.

21. The method according to claim 19 including the further step of counting the number of captured white blood cells of a specific type.

22. The method according to claim 21 wherein said specific types of white blood cells include neutrophils, monocytes, lymphocytes, eosinophils, and basophils.

23. An optical bio-disc employed for performing the white blood cell analysis in accordance with the methods recited in any one of claims 19 to 22.

24. A method of conducting an assay employing an optical disc and disc drive, said method comprising the steps of:
providing a sample of cells on a disc surface;
loading the disc into an optical reader;
rotating the optical disc;
directing an incident beam of electromagnetic radiation to the capture zone;
detecting a beam of electromagnetic radiation formed after interacting with the disc and the sample of cells on the disc surface;
converting the detected beam into an output signal; and
analyzing the output signal to extract therefrom information relating to the number and type of cells captured at the capture zone.

25. The method according to claim 24 further comprising the step of staining the sample of cells with a dye that absorbs light at a pre-determined wavelength.

26. The method according to claim 25 wherein said pre-determined wavelength is at or near the wavelength of the beam of electromagnetic radiation.

27. The method according to claim 25 wherein said dye absorbs light within the infrared spectral range.

28. The method according to claim 25 wherein said dye is a near-infrared absorbing stain.

29. The method according to claim 25 wherein said dye absorbs light within the ultra violet spectral range.

30. The method according to claim 25 wherein said dye absorbs light within the visible spectral range.

31. The method according to any of the claims 26 to 30 wherein said beam of electromagnetic radiation has a wavelength within 10 nm of the absorbance wavelength of said dye.

32. The method according to either claim 27 or 28 wherein said dye is selected from the group comprising LI-COR IRDye38, TO-PRO-5-iodide, IR-780 iodide, Laser Pro IR, dd-007, Zynostain, idocyanine green, copper phthalocyanine, 3,3′-diethylthiatricarbocyanine iodide (DTTCI), 3,3′-diethyloxatricarbocyanine iodide (DOTCI), 3,3′-diethylthiadicarbocyanine iodide (DTDCI), and 3,3′-diethyloxadicarbocyanine iodide (DODCI).

33. The method according to claim 25 wherein said dye labels pre-determined compartments of the sample of cells on the disc surface.

34. The method according to claim 33 wherein said pre-determined compartments include the nucleus, nucleolus, nuclear envelope, golgi apparatus, endoplasmic reticulum, mitochondria, vacuoles, peroxisomes, microtubules, centrioles, ribosomes, cell membrane, and cell wall.

35. The method according to claim 24 wherein said step of providing a sample of cells on a disc surface is performed by smearing the sample of cells to thereby create a monolayer of cells on the disc surface.

36. The method according to claim 18 wherein said cross-linker binds with oligossacharides on surface of cells.

37. The method according to claim 36 wherein said cross-linker is lectin.

38. A method of using the disc made according to any of the claims 18, 36, or 37; said method of using comprising:
depositing a sample comprising white blood cells into said channel;
allowing said white blood cells to bind to said cross-linker within said capture zones;
removing unbound cells from the capture zones;
loading the disc into an optical reader;
directing an incident beam of electromagnetic radiation to the capture zone;
detecting a beam of electromagnetic radiation formed after interacting with the disc and the sample of cells on the disc surface;
converting the detected beam into an output signal; and
analyzing the output signal to extract therefrom information relating to the number and type of cells captured at the capture zone.

39. The method according to claim 38 further comprising the step of staining the sample of cells with a stain that absorbs light at a pre-determined wavelength.

40. The method according to claim 39 wherein said pre-determined wavelength is at or near the wavelength of the beam of electromagnetic radiation.
